(19) United States
(12) Patent Application Publication
(10) Pub. No.: US 2003/0114457 A1
(43) Pub. Date: Jun. 19, 2003

(54) 2- [5- (5-carbamimidoyl-1H-heteroaryl)-6-hydroxybiphenyl-3-yl]-succinic acid derivatives as factor viia inhibitors

(75) Inventors: Huiyong Hu, Foster City, CA (US); John Hendrix, San Francisco, CA (US); Aleksandr Kolesnikov, San Francisco, CA (US); Roopa Rai, San Carlos, CA (US); William Dvorak Shrader, Belmont, CA (US); David Sperandio, Mountain View, CA (US); Wendy Young, San Mateo, CA (US); Steve Torkelson, San Mateo, CA (US)

Correspondence Address: AXYS PHARMACEUTICALS, INC., 180 KIMBALL WAY, SOUTH SAN FRANCISCO, CA, 94080, US

(73) Assignee: Axys Pharmaceuticals, Inc., South San Francisco, CA (US)

(21) Appl. No.: 10/190,147

(22) Filed: Jul. 3, 2002

Related U.S. Application Data
(60) Provisional application No. 60/303,953, filed on Jul. 9, 2001.
(60) Provisional application No. 60/351,054, filed on Jan. 22, 2002.

(51) Int. Cl. A61K 31/53; A61K 31/519; A61K 31/52; A61K 31/502

(52) U.S. Cl. 514/243; 514/249; 514/248; 514/263.1; 544/277; 544/280; 544/350; 544/184; 544/183; 514/265.1; 544/236

(57) ABSTRACT

The present invention relates to novel inhibitors of Factors VIIa, IXa, Xa, XIa, in particular Factor VIIa, pharmaceutical compositions comprising these inhibitors, and methods for using these inhibitors for treating or preventing thromboembolic disorders. Processes for preparing these inhibitors are also disclosed.

### CROSS REFERENCE

This application claims priority under 35 U.S.C. 119(e) to U.S. Provisional Applications Serial Nos. 60/303953, filed on Jul. 9, 2001 and 60/351054, filed on Jan., 22, 2002, the disclosures of which are incorporated herein by reference in their entirety.

### BACKGROUND OF THE INVENTION

1. Field of invention

The present invention relates to novel inhibitors of Factors VIIa, IXa, Xa, XIa, in particular Factor VIIa, pharmaceutical compositions comprising these inhibitors, and methods for using these inhibitors for treating or preventing thromboembolic disorders. Processes for preparing these inhibitors are also disclosed.

2. State of the Art

Thrombosis results from a complex sequence of biochemical events, known as the coagulation cascade. A triggering event in coagulation is the binding of the serine protease Factor VIIa (FVIIa) found in the circulation, to tissue factor (TF), a receptor which is found on the surface of blood vessels after damage or inflammation. Once bound to TF, Factor VIIa catalyzes the formation of the serine protease Factor Xa, which subsequently forms the final protease in the cascade, thrombin.

The clinical manifestations of thrombosis range from acute myocardial infarction (AMI or heart attack) and unstable angina (UA) which occur in the key blood vessels of the heart (coronary vasculature) to deep vein thrombosis (DVT) which is the formation of blood clots in lower extremities which often follows orthopedic surgery on the hip and knee, as well as general abdominal surgery and paralysis. Formation of DVT is a risk factor for the development of pulmonary embolism (PE) in which part of a blood clot formed in the lower extremities, breaks off and travels to the lung where it blocks the flow of blood. The unpredictable development of PE often leads to a fatal outcome. Thrombosis can also be generalized systemically, with microclot formation occurring throughout the vascular system. This condition, known as disseminated intravascular coagulation (DIC), can be a consequence of certain viral diseases such as Ebola, certain cancers, and sepsis. Severe DIC can lead to a dramatic reduction in the coagulation factors due to the excessive activation of the clotting response which may result in multiple organ failure, hemorrhage and death.

The formation or embolization of blood clots in the blood vessels of the brain is the key event resulting in ischemic stroke. Triggering factors that lead to stroke are atrial fibrillation or abnormal rhythm of the atria of the heart and atherosclerosis followed by thrombosis in the main artery leading from the heart to the brain (carotid artery). Over 600,000 individuals suffer strokes each year in the U.S. Two-thirds of these stroke victims suffer some disability, and one-third suffer permanent and severe disability. Accordingly, there is a need for antithrombotic agents for the treatment of a variety of thrombotic conditions. The present invention fulfills this and related needs.

### SUMMARY OF THE INVENTION

In one aspect this invention is directed to a compound of Formula I:

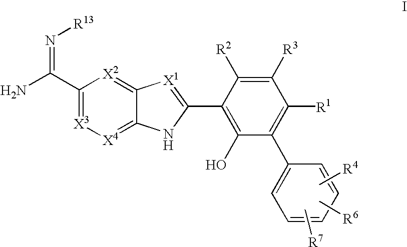

wherein:

X¹, X², X³, and X⁴ are independently —N— or —CR⁵— wherein R⁵ is hydrogen, alkyl, or halo with the proviso that not more than three of X¹, X², X3 and X⁴ are —N—;

R¹ and R² independently are hydrogen, alkyl, or halo;

R³ is —COOR⁹, -(alkylene)-COOR⁹, —CR⁸(COOR¹¹)alkylene-COOR⁹, or a group of formula (a):

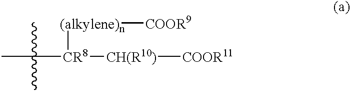

where:

n is 0 or 1;

R⁸ is hydrogen, alkyl, or hydroxy; and

R¹⁰ is hydrogen or alkyl; or

R⁸ and R¹⁰ together form a covalent bond;

R⁹ and R¹¹ are independently hydrogen, alkyl, haloalkyl, aryl, or aralkyl;

R⁴ is hydrogen, alkyl, alkylthio, halo, hydroxy, hydroxyalkyl, alkoxy, aminosulfonyl, alkylaminosulfonyl, dialkylaminosulfonyl, or nitro;

R⁶ is hydrogen, alkyl, or halo;

R⁷ is hydrogen, alkyl, cycloalkyl, alkylthio, halo, hydroxy, nitro, cyano, alkoxy, haloalkoxy, carboxy, alkoxycarbonyl, acylamino, alkylsulfonyl, arylsulfonyl, heteroarylsulfonyl, carbamimidoyl, hydroxycarbamimidoyl, alkoxycarbamimidoyl, alkylsulfonylamino, alkoxysulfonylamino, alkylsulfonylaminoalkyl, alkoxysulfonylaminoalkyl, heterocycloalkylalkylaminocarbonyl, hydroxyalkoxyalkylaminocarbonyl, haloalkyl, cyanoalkyl, alkoxyalkyl, hydroxyalkyl, carboxyalkyl, alkoxycarbonylalkyl, heterocycloalkylcarbonyl, heterocycloalkylcarbonylalkyl, heterocycloalkyl, heterocycloalkylalkyl, oxoheterocycloalkylalkyl, aminosulfonylalkyl, heteroaryl, heteroaralkyl, ureido, alkylureido, dialkylureido, ureidoalkyl, alkylureidoalkyl, dialkylureidoalkyl, thioureido, thioureidoalkyl, —COR¹² (where R¹² is alkyl or haloalkyl), -(alkylene)-COR¹² (where R¹² is alkyl or haloalkyl), aminocarbonyl, aminocarbonylalkyl, —CONR¹⁴R¹⁵ (where R¹⁴ is hydrogen or alkyl and R¹⁵ is alkyl, aryl, aralkyl, heteroaryl, or heteroaralkyl), -(alkylene)-CONR¹⁶R¹⁷ (where R¹⁶ is hydrogen or alkyl and R¹⁷ is alkyl, aryl, aralkyl, heteroaryl, or heteroaralkyl), amino, alkylamino, dialkylamino, —NR¹⁸R¹⁹ (where R¹⁸ is hydrogen or alkyl and R¹⁹ is aryl, aralkyl, heteroaryl, or heteroaralkyl), aminoalkyl, -(alkylene)-NR²⁰R²¹ (where R²⁰ is hydrogen or alkyl and R²¹ is alkyl, aryl, aralkyl, heteroaryl, or heteroaralkyl), aminosulfonyl, —SO₂NR²²R²³ (where R²² is hydrogen or alkyl and R²³ is alkyl, aryl, aralkyl, heteroaryl, or heteroaralkyl, or R²² and R²³ together with the nitrogen atom to which they are attached from heterocycloamino), -(alkylene)-SO₂NR²⁴R²⁵ (where R²⁴ is hydrogen or alkyl and R²⁵ is alkyl, aryl, aralkyl, heteroaryl, or heteroaralkyl or R²⁴ and R²⁵ together with the nitrogen atom to which they are attached from heterocycloamino), aminosulfonylamino, —NR²⁶SO₂NR²⁷R²⁸ (where R²⁶ and R²⁷ are independently hydrogen or alkyl, and R²⁸ is alkyl, aryl, aralkyl, heteroaryl, or heteroaralkyl or R²⁷ and R²⁸ together with the nitrogen atom to which they are attached from heterocycloamino), -(alkylene)-NR²⁹SO₂NR³⁰R³¹ (where R²⁹ and R³⁰ are independently hydrogen or alkyl, and R³¹ is hydrogen, alkyl, aryl, aralkyl, heteroaryl, or heteroaralkyl or R³⁰ and R³¹ together with the nitrogen atom to which they are attached from heterocycloamino), —CONH-(alkylene)-NR³²R³³ where R³² is hydrogen or alkyl and R³³ is alkyl), or -(alkenylene)-R³⁴ (where R³⁴ is alkoxy, carboxy, alkoxycarbonyl, amino, alkylamino, dialkylamino, acylamino, aminosulfonylamino, alkylaminosulfonylamino, alkylsulfonyl, aryl, heteroaryl, heterocycloalkyl, heterocycloalkylcarbonyl, aminocarbonyl, aminosulfonyl, —COR¹², —CONR¹⁴R¹⁵, —NR¹⁸R¹⁹, —SO₂NR²²R²³, or —NR²⁶SO₂NR²⁷R²⁸ where R¹², R¹⁴, R¹⁵, R¹⁸, R¹⁹, R²², R²³, R²⁶, R²⁷, and R²⁸ are as defined above); and

R¹³ is hydrogen, hydroxy, (C₁₋₁₀)alkoxy, —C(O)R³⁵ where R³⁵ is alkyl, aryl, haloalkyl, or cyanoalkyl, or —C(O)OR³⁶ where R³⁶ is alkyl, hydroxyalkyl, acyl, or haloalkyl; and individual isomers, mixture of isomers, or a pharmaceutically acceptable salt thereof, provided that when R⁷ is hydrogen, alkyl, halo, nitro, alkoxy, haloalkyl, carboxy, alkoxycarbonyl, amino, alkylamino, dialkylamino, —NR^{R}¹⁹ (where R¹⁸ is hydrogen or alkyl and R¹⁹ is aryl or aralkyl), pyrrolidinylcarbonyl, —SO₂NR²²R²³ (where R²² and R²³ are alkyl), carbamimidoyl, alkylsulfonylamino, alkylthio, ureido or —NHC(S)NH₂, and R³ is —COOR⁹, -(alkylene)-COOR⁹, —CR⁸(COOR¹¹)alkylene-COOR⁹, or a group of formula (a) where n is 0 or 1; R⁸ and R¹⁰ are independently hydrogen or alkyl, and R¹³ is hydrogen; then R⁴ is hydroxy or hydroxyalkyl.

Preferably,

X¹, X², X³, and X⁴ are independently —N— or —CR⁵— wherein R is hydrogen, alkyl, or halo with the proviso that not more than three of X¹, X², X³ and X⁴ are —N—;

R¹ and R² independently are hydrogen, alkyl, or halo;

R³ is —COOR⁹, -(alkylene)-COOR⁹ where R⁹ is hydrogen or alkyl, or a group of formula (a):

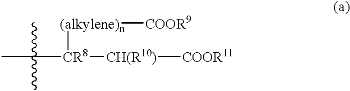

where:

n is 0 or 1;

R⁸ is hydrogen, alkyl, or hydroxy; and

R¹⁰ is hydrogen or alkyl; or

R⁸ and R¹⁰ together form a covalent bond;

R⁹ and R¹¹ are independently hydrogen or alkyl;

R⁴ is hydrogen, alkyl, alkylthio, halo, hydroxy, hydroxyalkyl, alkoxy, or nitro;

R⁶ is hydrogen, alkyl, or halo;

R⁷ is hydrogen, alkyl, halo, hydroxy, nitro, cyano, alkoxy, haloalkyl, haloalkoxy, —COR¹² (where R¹² is alkyl), aminocarbonyl, hydroxyalkyl, carboxy, carboxyalkyl, amino, alkylamino, dialkylamino, heterocycloalkylalkylaminocarbonyl, cyanoalkyl, aminocarbonylalkyl, alkoxyalkyl, hydroxyalkoxyalkylaminocarbonyl, heterocycloalkylcarbonyl, heterocycloalkylalkyl, carbamimidoyl, aminosulfonylamino, alkylaminosulfonylamino, alkylsulfonylamino, alkylthio, aminoalkyl, ureidoalkyl, heteroaryl, or ureido provided that when R⁷ is hydrogen, alkyl, halo, nitro, alkoxy, haloalkyl, carboxy, amino, alkylamino, dialkylamino, heterocycloalkylcarbonyl, carbamimidoyl, alkylsulfonylamino, alkylthio, or ureido, then R⁴ is hydroxy or hydroxyalkyl; or a pharmaceutically acceptable salt thereof.

In a second aspect, this invention is directed to a pharmaceutical composition comprising a pharmaceutically acceptable carrier and a therapeutically effective amount of a compound of Formula I or a pharmaceutically acceptable salt thereof. The pharmaceutical composition can contains individual stereoisomer or mixture of stereoisomers of a compound of Formula I.

In a third aspect, this invention is directed to a method of treating a disease in an animal mediated by Factors VIIa, IXa, Xa and/or XIa, preferably VIIa, which method comprises administering to said animal a pharmaceutical composition comprising a pharmaceutically acceptable carrier and a therapeutically effective amount of a compound of Formula I or a pharmaceutically acceptable salt thereof. The pharmaceutical composition can contains individual stereoisomer or mixture of stereoisomers of a compound of Formula I. Preferably, the disorder is a thromboembolic disorder or cancer, more preferably a thromboembolic disorder.

In a fourth aspect, this invention is directed to a method of treating a thromboembolic disorder in an animal which method comprises administering to said animal a pharmaceutical composition comprising a pharmaceutically acceptable carrier and a therapeutically effective amount of a compound of Formula I or a pharmaceutically acceptable salt thereof in combination with another anticoagulant agent(s) independently selected from a group consisting of a thrombin inhibitor, factor IXa inhibitor, factor Xa inhibitor, Aspirin®, and Plavis®.

In a fifth aspect, this invention is directed to a method for inhibiting the coagulation of a biological sample (e.g., stored blood products and samples) comprising the administration of a compound of Formula I or a pharmaceutically acceptable salt thereof.

In a sixth aspect, this invention directed to the use of a compound of Formula I or a pharmaceutically acceptable salt thereof in the preparation of a medicament for use in the treatment of a thromboembolic disorder or cancer in an animal. Preferably, the disorder is a thromboembolic disorder.

In a seventh aspect, this invention is directed to an intermediate of Formula II:

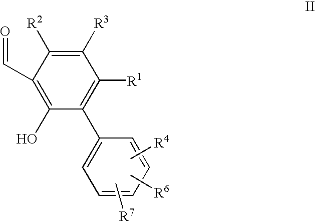

wherein R¹, R², R³, R⁴, R⁶, and R⁷ are as defined for compounds of Formula I above.

In an eighth aspect, this invention is directed to a process of preparing a compound of Formula I where X¹ is —N— comprising reacting a compound of Formula II with a compound of Formula III:

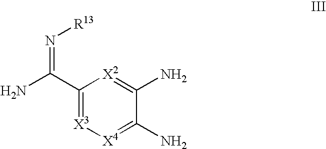

where

R¹³ is hydrogen;

optionally modifying any of the R¹, R², R³, R⁴, R⁶, R⁷, and R¹³ groups;

optionally isolating individual isomers;

optionally preparing an acid addition salt; and

optionally preparing a free base.

In a ninth aspect, this invention is directed to a process of preparing a compound of Formula I where X¹ is —CH— and R¹³ is hydrogen, comprising reacting a compound of Formula IV:

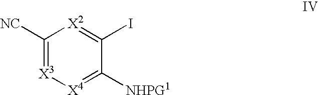

where X², X³, X⁴ are as defined in the Summary of the Invention and PG¹ is a suitable amino protecting group;

with a compound of Formula V

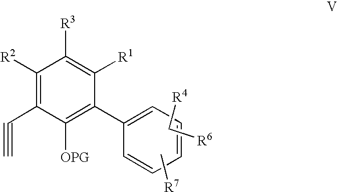

where R¹, R², R³, R⁴, R⁶, and R⁷ are as defined in the Summary of the Invention and PG is a suitable oxygen protecting group; to give a compound of Formula VI:

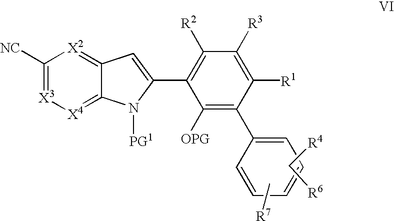

optionally removing the amino and/or hydroxy protecting group;

converting the cyano to a carbamimidoyl group;

optionally removing the amino and/or hydroxy protecting group;

optionally modifying any of the R¹, R², R³, R⁴, R⁶, R⁷, and R¹³ groups;

optionally isolating individual isomers;

optionally preparing an acid addition salt; and

optionally preparing a free base.

### DETAILED DESCRIPTION OF THE INVENTION

### Definitions

The following terms, as used in the present specification and claims, are intended to have the meaning as defined below, unless indicated otherwise.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkylene” means a linear saturated divalent hydrocarbon radical of one to six carbon atoms or a branched saturated divalent hydrocarbon radical of three to six carbon atoms e.g., methylene, ethylene, propylene, 1-methylpropylene, 2-methylpropylene, butylene, pentylene, and the like.

“Alkenylene” means a linear divalent hydrocarbon radical of two to six carbon atoms or a branched saturated divalent hydrocarbon radical of three to six carbon atoms containing one or two double bonds e.g., ethenylene, propenylene, 2-methylpropenylene, and the like.

“Alkylthio” means a radical —SR where R is alkyl as defined above, e.g., methylthio, ethylthio, propylthio (including all isomeric forms), butylthio (including all isomeric forms), and the like.

“Amino” means a radical —NH₂.

“Alkylamino” means a radical —NHR where R is alkyl as defined above, e.g., methylamino, ethylamino, n-, iso-propylamino, n-, iso-, tert-butylamino, methylamino-N-oxide, and the like.

“Acyl” means a radical —COR′ where R′ is alkyl or haloalkyl as defined herein, e.g., acetyl, trifluoroacetyl, and the like.

“Acylamino” means a radical —NRCOR′ where R is hydrogen or alkyl and R′ is alkyl or haloalkyl as defined herein, e.g., acetylamino, trifluoroacetylamino, and the like.

“Aminosulfonyl” means a radical —SO₂NH₂.

“Aminosulfonylalkyl” means a radical -(alkylene)-SO₂NH₂ e.g., aminosulfonylmethyl, and the like.

“Aminosulfonylamino” means a radical —NHSO₂NH₂.

“Alkylaminosulfonylamino” means a radical —NRSO₂NHR′ where R is hydrogen or alkyl, and R′ is alkyl as defined above, e.g., methylaminosulfonylamino, ethylaminosulfonylamino, n- or iso-propylaminosulfonylamino, and the like.

“Alkylaminosulfonyl” means a radical —SO₂NHR′ where R′ is alkyl as defined above, e.g., methylaminosulfonyl, ethylaminosulfonyl, n- or iso-propylaminosulfonyl, and the like.

“Alkylsulfonyl” means a radical —SO₂R where R is alkyl as defined above, e.g., methylsulfonyl, ethylsulfonyl, n- or iso-propylsulfonyl, and the like.

“Alkylsulfonylamino” means a radical —NHSO₂R where R is alkyl as defined above, e.g., methylsulfonylamino, ethylsulfonylamino, n- or iso-propylsulfonylamino, and the like.

“Alkylsulfonylaminoalkyl” means a radical -(alkylene)-NHSO₂R where R is alkyl as defined above, e.g., methylsulfonylaminomethyl, ethylsulfonylaminomethyl, n- or iso-propylsulfonylaminoethyl, and the like.

“Alkoxysulfonylamino” means a radical —NHSO₂R where R is alkoxy as defined herein, e.g., methoxysulfonylamino, ethoxysulfonylamino, and the like.

“Alkoxysulfonylaminoalkyl” means a radical -(alkylene)-NHSO₂R where R is alkoxy as defined herein, e.g., methoxysulfonylaminomethyl, ethoxysulfonylaminomethyl, and the like.

“Alkoxy” means a radical —OR where R is alkyl as defined above, e.g., methoxy, ethoxy, propoxy, or 2-propoxy, n-, iso-, or tert-butoxy, and the like.

“Alkoxycarbonyl” means a radical —COOR where R is alkyl as defined above, e.g., methoxycarbonyl, ethoxycarbonyl, and the like.

“Alkoxycarbonylalkyl” means a radical -(alkylene)-COOR where R is alkyl as defined above, e.g., methoxycarbonylmethyl, ethoxycarbonylmethyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Aminoalkyl” means a linear monovalent hydrocarbon radical of one to six carbon atoms or a branched monovalent hydrocarbon radical of three to six carbons substituted with at least one, preferably one or two, —NHR where R is hydrogen or —COR^{a} where R^{a} is alkyl, e.g., aminomethyl, methylaminoethyl, 1,3-diaminopropyl, acetylaminopropyl, and the like.

“Aminocarbonyl” means a radical —CONH₂.

“Aminocarbonylalkyl” means a radical -(alkylene)-CONH₂, e.g., aminocarbonylmethyl, aminocarbonylethyl, 1-, 2-, or 3-aminocarbonylpropyl, and the like.

“Alkylureido” means a radical —NRCONHR′ where R is hydrogen or alkyl and R′ is alkyl, e.g., methylureidomethyl, and the like.

“Alkylureidoalkyl” means a radical -(alkylene)-NRCONHR′ where R is hydrogen or alkyl and R′ is alkyl, e.g., methylureidomethyl, and the like.

“Aryl” means a monovalent monocyclic or bicyclic aromatic hydrocarbon radical of 6 to 12 ring atoms, and optionally substituted independently with one or more substituents, preferably one, two, or three substituents, selected from alkyl, haloalkyl, alkoxy, alkylthio, halo, nitro, —COR (where R is alkyl), cyano, amino, alkylamino, dialkylamino, hydroxy, carboxy, or —COOR where R is alkyl. Representative examples include, but are not limited to, phenyl, biphenyl, 1-naphthyl, and 2-naphthyl and the derivatives thereof.

“Arylsulfonyl” means a radical —SO₂R where R is aryl as defined above, e.g., phenylsulfonyl, and the like.

“Aralkyl” means a radical -(alkylene)-R where R is an aryl group as defined above e.g., benzyl, phenylethyl, 3-(3-chlorophenyl)-2-methylpentyl, and the like.

“Alkoxycarbamimidoyl” means a radical —C(═NH)NHOR or —C(═NOR)NH₂ where R is alkyl as defined above, e.g., methoxycarbamimidoyl.

“Cycloalkyl” means a cyclic saturated monovalent hydrocarbon radical of three to six carbon atoms, which is substituted with —NR^{a}R^{b} (where R^{a} is hydrogen or alkyl and R^{b} is hydrogen, alkyl, —SO₂R, —C(O)NR′R″, —C(S)NR^{c}R^{d}, —C(═NH)NR^{c}R^{d}, —SO₂NR^{e}R^{f} where R is alkyl or alkoxy, R′ and RR″ are independently hydrogen or alkyl, R^{c} is hydrogen or alkyl, R^{d} is hydrogen, alkyl, hydroxy or alkoxy, R^{e} is hydrogen or alkyl and R^{f} is hydrogen, alkyl or R^{e} and R^{f} form heterocycloamino), hydroxy, alkoxy, aminosulfonyl, alkylaminosulfonyl, dialkylaminosulfonyl, e.g., cyclopropyl, cyclobutyl, and the like, preferably cyclopropyl.

“Carboxyalkyl” means a radical -(alkylene)-COOH, e.g., carboxymethyl, carboxyethyl, 1-, 2-, or 3-carboxypropyl, and the like.

“Carbamimidoyl” means a radical —C(═NH)NH₂, or a protected derivative thereof.

“Cyanoalkyl” means a radical -(alkylene)-CN, e.g., cyanomethyl, cyanoethyl, cyanopropyl, and the like.

“Dialkylamino” means a radical —NRR′ where R and R′ are independently alkyl as defined above, e.g., dimethylamino, diethylamino, methylpropylamino, methylethylamino, n-, iso-, or tert-butylamino, and the like.

“Dialkylaminosulfonyl” means a radical —SO₂NRR′ where R and R′ are independently alkyl as defined above, e.g., dimethylaminosulfonyl, methylethylaminosulfonyl, and the like.

“Dialkylureido” means a radical —NRCONR′R″ where R is hydrogen or alkyl and R′ and R″ are independently alkyl, e.g., dimethylureido, and the like.

“Dialkylureidoalkyl” means a radical -(alkylene)-NRCONR′R″ where R is hydrogen or alkyl and R′ and R″ are independently alkyl, e.g., dimethylureidomethyl, and the like.

“Halo” means fluoro, chloro, bromo, and iodo, preferably fluoro or chloro.

“Haloalkyl” means alkyl substituted with one or more halogen atoms, preferably one to three halogen atoms, preferably fluorine or chlorine, including those substituted with different halogens, e.g., —CH₂Cl, —CF₃, —CHF₂, and the like.

“Haloalkoxy” means a radical —OR where R is haloalkyl as defined above, e.g., —OCH₂Cl, —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxyalkylaminocarbonyl” means a radical —CONH-(alkylene)-O-(alkylene)OH where alkylene is as defined above, e.g., —CONH—(CH₂)₂—O—(CH₂)₂OH and the like.

“Heterocycloalkyl” means a saturated or unsaturated monovalent cyclic group of 3 to 8 ring atoms in which one or two ring atoms are heteroatoms selected from N, O, or S(O)n, where n is an integer from 0 to 2, the remaining ring atoms being C. The heterocycloalkyl ring may be optionally substituted with one or more substituents, preferably one or two substituents, independently selected from alkyl, aryl, heteroaryl, aralkyl, heteroaralkyl, halo, haloalkyl, hydroxy, hydroxyalkyl, alkoxy, alkoxyalkyl, halo, cyano, carboxy, or —COOR where R is alkyl as define above or a protected derivative thereof. More specifically the term heterocycloalkyl includes, but is not limited to, pyrrolidino, piperidino, morpholino, piperazino, tetrahydropyranyl, and thiomorpholino.

“Heterocycloalkylcarbonyl” means a radical —COR where R is heterocycloalkyl as defined above. More specifically the term heterocycloalkylcarbonyl includes, but is not limited to, 1-pyrrolidinocarbonyl, 1-piperidinocarbonyl, 4-morpholinocarbonyl, 1-piperazinocarbonyl, 2-tetrahydropyranylcarbonyl, and 4-thiomorpholinocarbonyl, and the derivatives thereof.

“Heterocycloalkylcarbonylalkyl” means a radical -(alkylene)-COR where R is heterocycloalkyl as defined above. More specifically the term heterocycloalkylcarbonyl includes, but is not limited to, 1-pyrrolidinocarbonylmethyl, 1-piperidinocarbonylmethyl, 4-morpholinocarbonylethyl, 1-piperazinocarbonylmethyl, and the derivatives thereof.

“Heterocycloalkylalkyl” means a radical -(alkylene)-R where R is heterocycloalkyl as defined above. More specifically the term heterocycloalkylalkyl includes, but is not limited to, pyrrolidin-1-ylmethyl, piperidin-1-ylmethyl, 2-morpholin-1-ylethyl, piperazin-1-ylethyl, and the derivatives thereof.

“Heterocycloalkylalkylaminocarbonyl” means a radical —CONH-(alkylene)-R where R is heterocycloalkyl as defined above. More specifically the term heterocycloalkylalkylamino-carbonyl includes, but is not limited to, 1-pyrrolidinoethyl-aminocarbonyl, 1-piperidinoethyl-aminocarbonyl, 4-morpholinoethylcarbonyl, 1-piperazinoethylaminocarbonyl, and 4-thiomorpholinopropylaminocarbonyl, and the derivatives thereof.

“Heteroaryl” means a monovalent monocyclic or bicyclic aromatic radical of 5 to 10 ring atoms containing one or more, preferably one or two ring heteroatoms selected from N, O, or S, the remaining ring atoms being carbon. The heteroaryl ring is optionally substituted with one or more substituents, preferably one or two substituents, independently selected from alkyl, haloalkyl, alkoxy, alkylthio, halo, nitro, cyano, amino, alkyl or dialkylamino, hydroxy, carboxy, or —COOR where R is alkyl as define above. More specifically the term heteroaryl includes, but is not limited to, pyridyl, pyrrolyl, imidazolyl, thienyl, furanyl, indolyl, quinolyl, pyrazine, pyrimidine, pyradizine, oxazole, isooxazolyl, benzoxazole, quinoline, isoquinoline, benzopyranyl, and thiazolyl.

“Heteroarylsulfonyl” means a radical —SO₂R where R is heteroaryl as defined above, e.g., pyridylsulfonyl, furanylsulfonyl, and the like.

“Heteroaralkyl” means a radical -(alkylene)-R where R is a heteroaryl group as defined above e.g., pyridylmethyl, furanylmethyl, indolylmethyl, pyrimidinylmethyl, and the like.

“Heterocycloamino” means a saturated or unsaturated monovalent cyclic group of 3 to 8 ring atoms in which one or two ring atoms are heteroatoms selected from N, O, or S(O)n, where n is an integer from 0 to 2, the remaining ring atoms being C provided that at least one of the heteroatom is nitrogen and wherein one or two carbon atoms are optionally replace by a carbonyl group. The heterocycloamino ring may be optionally substituted with one or more substituents, preferably one or two substituents, independently selected from alkyl, hydroxy, hydroxyalkyl, alkoxy, alkoxyalkyl, halo, haloalkyl, aryl, heteroaryl, aralkyl, heteroaralkyl, haloalkyl, halo, cyano, carboxy, or —COOR where R is alkyl as define above. More specifically the term heterocycloamino includes, but is not limited to, pyrrolidino, piperidino, piperazino, and thiomorpholino, and the derivatives thereof.

“Hydroxycarbamimidoyl” means a radical —C(═NH)NHOH or —C(═NOH)NH₂.

The present invention also includes the prodrugs of compounds of Formula I. The term prodrug is intended to represent covalently bonded carriers, which are capable of releasing the active ingredient of Formula I, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I include compounds wherein a hydroxy, amidino, guanidino, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy functional groups in compounds of Formula I and the like. Prodrugs of compounds of Formula I are also within the scope of this invention.

The present invention also includes (derivatives and protected derivatives of compounds of Formula I. For example, when compounds of Formula I contain an oxidizable nitrogen atom (e.g., when a compound of Formula I contains a pyridine, amino, alkylamino, piperidino, piperazino, morpholino, or dialkylamino group), the nitrogen atom can be converted to an N-oxide by methods well known in the art.

Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. Many geometric isomers of olefins, C═C double bonds, and the like can be present in the compounds described herein, and all such stable isomers are contemplated in the present invention. Cis and trans geometric isomers of the compounds of the present invention are described and may be isolated as a mixture of isomers or as separated isomeric forms. All chiral, enantiomeric, diastereomeric, racemic forms and all geometric isomeric forms of a structure (representing a compound of Formula I) are intended, unless the specific stereochemistry or isomeric form is specifically indicated.

Certain compounds of Formula I exist in tautomeric equilibrium. Compounds of Formula I, which exist as tautomers are named, illustrated or otherwise described in this application as one possible tautomer. However, it is to be understood that all possible tautomers are meant to be encompassed by such names, illustrations and descriptions and are within the scope of this invention. For example, in compound of Formula I, the group —C(═NR¹³)NH₂ can tautomerize to —C(═NH)NHR¹³ group. Additionally, as used herein the terms alkyl includes all the possible isomeric forms of said alkyl group albeit only a few examples are set forth. Furthermore, when the cyclic groups such as aryl, heteroaryl, heterocycloalkyl are substituted, they include all the positional isomers albeit only a few examples are set forth.

“Oxoheterocycloalkyl” means a saturated or unsaturated (provided that it is not aromatic) monovalent cyclic group of 3 to 8 ring atoms in which one or two ring atoms are heteroatoms selected from N, O, or S(O)n, where n is an integer from 0 to 2, the remaining ring atoms being C wherein one or two of the carbon atoms is/are replaced with an oxo (C═O) group. The oxoheterocycloalkyl ring may be optionally substituted with one or more substituents, preferably one or two substituents, independently selected from alkyl, aryl, heteroaryl, aralkyl, heteroaralkyl, haloalkyl, halo, hydroxy, hydroxyalkyl, alkoxyalkyl, aminoalkyl, alkoxy, cyano, carboxy, or —COOR where R is alkyl as define above. More specifically the term heterocycloalkyl; includes, but is not limited to, 2 or 3-oxopyrrolidin-1-yl, 2, 3, or 4-oxopiperidino, 3-oxomorpholino, 2-oxo-piperazino, 2-oxotetrahydropyranyl, 3-oxothiomorpholino, 2-imidazolidone, and the derivatives thereof.

“Oxoheterocycloalkylalkyl” means a radical -(alkylene)-R where R is a oxoheterocycloalkylalkyl group as defined above e.g., More specifically the term oxoheterocycloalkylalkyl; includes, but is not limited to, 2 or 3-oxopyrrolidin-1-yl-(methyl, ethyl, or propyl), 2, 3, or 4-oxopiperidin-1-yl-(methyl, ethyl, or propyl), 3-oxomorpholin4-yl-(methyl, ethyl, or propyl), 2-oxopiperazin-1-yl-(methyl, ethyl, or propyl), 2-oxotetrahydro-pyran-3-yl-(methyl, ethyl, or propyl), 3-oxothiomorpholin-4-yl-(methyl, ethyl, or propyl), 2-imidazolidon-1-yl-(methyl, ethyl, or propyl), and the derivatives thereof.

“Optional” or “optionally” means that the subsequently described event or circumstance may but need not occur, and that the description includes instances where the event or circumstance occurs and instances in which it does not. For example, “heterocycloalkyl group optionally mono- or di-substituted with an alkyl group” means that the alkyl may but need not be present, and the description includes situations where the heterocycloalkyl group is mono- or disubstituted with an alkyl group and situations where the heterocycloalkyl group is not substituted with the alkyl group.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A “therapeutically effective amount” means the amount of a compound of Formula I that, when administered to a mammal for treating a disease, is sufficient to effect such treatment for the disease. The “therapeutically effective amount” will vary depending on the compound, the disease and its severity and the age, weight, etc., of the mammal to be treated.

“Thioureido” means a radical —NRC(S)NR′R″ where R, R′, and R″ are independently hydrogen or alkyl.

“Thioureidoalkyl” means a radical -(alkylene)-NRC(S)NR′R″ where alkylene is as defined above. Representative examples include but are not limited to thioureidomethyl, thioureidoethyl, and the like.

“Ureido” means a radical —NHCONH₂.

“Ureidoalkyl” means a radical -(alkylene)-NHCONH₂ where alkylene is as defined above. Representative examples include but are not limited to ureidomethyl, ureidoethyl, and the like.

Numbering of the compounds of Formula I:

The compounds of the present invention are numbered as follows:

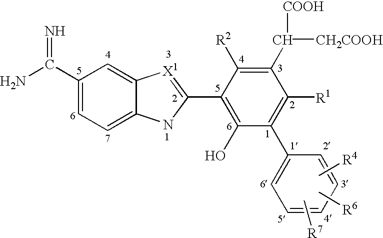

Representative compounds of Formula I are shown in Table I-V below:

TABLE I

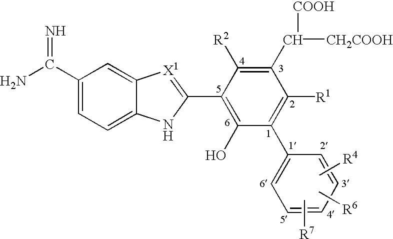

| Cpd # | X¹ | R¹ | R² | R⁴ | R⁶ | R⁷ |
|---|---|---|---|---|---|---|
| 1 | N | H | H | H | H | 3′-OCHF₂ |
| 2 | N | H | H | H | H | 3′-COCH₃ |
| 3 | N | H | H | H | H | 3′-OH |
| 4 | N | H | H | 2′-OH | H | H |
| 5 | N | H | H | H | H | 3′-CONH₂ |
| 6 | N | H | H | H | H | 3′-CN |
| 7 | N | H | H | 2′-OH | H | 5′-F |
| 8 | N | H | H | 2′-OH | H | 5′-Cl |
| 9 | N | H | H | 2′-CH₂OH | H | H |
| 10 | CH | H | H | 2′-OH | H | H |
| 11 | N | H | H | 2′-OH | H | 5′-COOH |
| 12 | N | H | H | 2′-OH | H | 5′-OH |
| 13 | N | H | H | 2′-OCH₃ | H | 5′-CN |
| 14 | N | H | H | 2′-OCH₃ | H | 5′-CONH₂ |
| 15 | N | H | H | 2′-OH | H | 6′-OH |
| 16 | N | H | H | 2′-OH | H | 5′-NO₂ |
| 17 | N | H | H | H | H | 2′-CN |
| 18 | N | H | H | H | H | 3′-CH₂OH |
| 19 | N | H | H | 2′-OH | H | 5′-CN |
| 20 | N | H | H | 2′-OH | H | 5′-CONH₂ |
| 21 | N | H | H | 2′-OH | 3′-Br | 6′-OH |
| 22 | N | H | H | 2′-OH | H | 5′-NHSO₂CH₃ |
| 23 | N | H | H | 2′-OH | H | 5′-CH(CH₃)₂ |
| 24 | N | H | H | 2′-OH | H | 5′-CH₂NH₂ |
| 25 | N | H | H | 2′-OH | H | 5′-CH₂NHCONH₂ |
| 26 | N | H | H | 2′-OH | H | 5′-imidazol-2-yl |
| 27 | N | H | H | 2′-OH | H | 5′-NH₂ |
| 28 | N | H | H | 2′-OH | H | 5′-NHCONH₂ |
| 29 | N | H | H | 2′-OH | H | 5′-CONH(CH₂)_{2—}morpho- |
| | | | | | | lin-4-yl |
| 30 | N | H | H | 2′-OH | 3′-Br | 5′-CH₂CN |
| 31 | N | H | H | 2′-OH | H | 5′-(CH₂)₂CN |
| 32 | N | H | H | 2′-OH | 3′-Br | 5′-CH₂COOH |
| 33 | N | H | H | 2′-OH | H | 5′-(CH₂)₂COOH |
| 34 | N | H | H | H | H | 2′-COCH₃ |
| 35 | N | H | H | 2′-OH | 3′-Br | 5′-CH₂CONH₂ |
| 36 | CH | H | H | 2′-OH | H | 5′-CONH₂ |
| 37 | N | H | H | 2′-OH | 3′-Cl | 5′-Cl |
| 38 | N | H | H | 2′-OH | H | 5′-CONH(CH₂)₂O(CH₂)₂OH |
| 39 | N | H | H | 2′-OH | 4′-Cl | 6′-Cl |
| 40 | N | H | H | 2′-OH | H | 5′-NHSO₂N(CH₃)₂ |
| 41 | N | H | H | 2′-OH | 3′-Br | 5′-Cl |
| 42 | N | H | H | 2′-OH | H | 5′-CO(4-methylpiperazin-1-yl) |
| 43 | N | H | H | 2′-OH | H | 5′-CH₂(4-methyl- |
| | | | | | | piperazin-1-yl) |
| 44 | N | H | H | 2′-OH | H | 5′-C(═NH)NH₂ |
| 45 | N | H | H | 2′-OH | H | 5′-CONH(CH₂)₂N(CH₃)₂ |
| 46 | N | H | H | 2′-OH | H | 5′-CH₂OH |
| 47 | N | H | H | 2′-OH | H | 5′-CH₂NHCONHCH₃ |
| 48 | N | H | H | H | H | 3′-SO₂NH₂ |
| 49 | N | H | H | 2′-OH | H | 5′-NHSO₂N(CH₃)₂ |
| 50 | CH | H | H | H | H | 3′-NHCONH₂ |

TABLE II

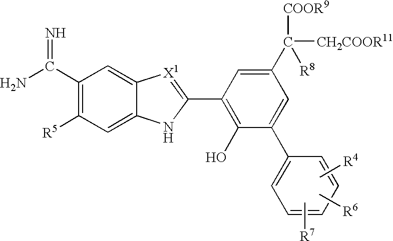

| Cpd # | X¹ | R⁵ | R⁴ | R⁶ | R⁷ | R⁸ | R⁹ | R¹¹ |
|---|---|---|---|---|---|---|---|---|
| 1 | N | F | 2′-OH | H | H | H | H | H |
| 2 | CH | Cl | 2′-OH | H | H | H | H | H |
| 3 | N | H | 2′-OH | 5′-F | H | H | CH₂CH₃ | CH₂CH₃ |
| 4 | N | F | 2′-OH | 5′-F | H | H | H | H |
| 5 | N | H | 2′-OH | 5′-F | H | CH₃ | H | H |
| 6 | N | H | 2′-OH | 5′-F | H | H | CH₂CH₃ | H |
| 7 | N | H | 2′-OH | 5′-F | H | H | H | CH₂CH₃ |

TABLE III

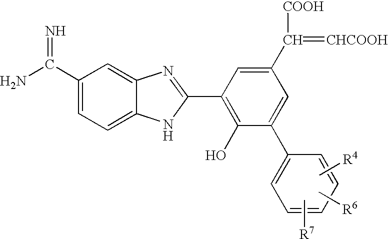

| | Isomerism around carbon- | | | |
|---|---|---|---|---|
| Cpd # | carbon double bond | R⁴ | R⁶ | R⁷ |
| 1 | Cis | 2′-OCH₃ | 5′F | H |
| 2 | Cis | 2′-OH | 5′-F | H |
| 3 | Trans | 2′-OH | 5′-F | H |

TABLE IV

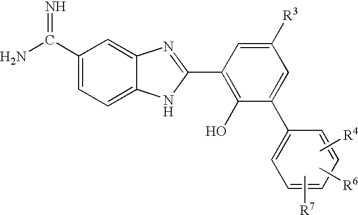

| Cpd # | R³ | R⁴ | R⁶ | R⁷ |
|---|---|---|---|---|
| 1 | —(CH₂)₂CO₂H | 2′-OH | 5′-F | H |
| 2 | —(CH₂)₂CO₂CH₃ | 2′-OH | 5′-F | H |
| 3 | —CH₂CO₂CH₃ | 2′-OH | 5′-F | H |
| 4 | —CH₂CO₂H | 2′-OH | H | H |
| 5 | —CH₂CO₂H | 2′-OH | 5′-F | H |

TABLE V

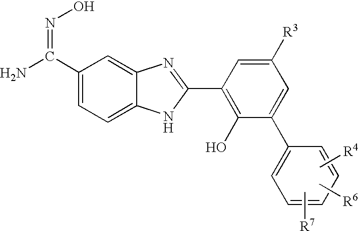

| Cpd # | R³ | R⁴ | R⁶ | R⁷ |
|---|---|---|---|---|
| 1 | —CH(COOH)CH₂CO₂H | 2′-OH | 5′-F | H |
| 2 | —CH(CO₂Et)CH₂CO₂Et | 2′-OH | 5′-F | H |
| 3 | —CH(COOH)CH₂CO₂H | 2′-OH | 5′-NHSO₂CH₃ | H |
| 4 | —CH(CO₂Et)CH₂CO₂Et | 2′-OH | 5′-CH₂NHCONH₂ | H |
| 6 | —CH₂COOH | 2′-OH | 5′-F | H |
| 7 | —CH(CO₂Et)CH₂CO₂Et | 2′-OH | 5′-CH₂OH | H |
| 8 | —CH(CO₂Me)CH₂CO₂Me | 2′-OH | 5′-CONH₂ | H |
| 9 | —CH(COOH)CH₂CO₂H | 2′-OH | 5′-CH₂NHCONH₂ | H |

and are named as:

2-[5-(5-carbamimidoyl-1H-benzoimidazol-2-yl)-3′-(1,1-difluoro-methoxy)-6-hydroxy-biphenyl-3-yl]-succinic acid;

2-[3′-acetyl-5-(5-carbamimidoyl-1H-benzoimidazol-2-yl)-6-hydroxy-biphenyl-3-yl]-succinic acid;

2-[5-(5-carbamimidoyl-1H-benzoimidazol-2-yl)-6,3′-dihydroxy-biphenyl-3-yl]-succinic acid;

2-[5-(5-carbamimidoyl-1H-benzoimidazol-2-yl)-6,2′-dihydroxy-biphenyl-3-yl]-succinic acid;

2-[5-(5-carbamimidoyl-1H-benzoimidazol-2-yl)-3′-aminocarbonyl-6-hydroxy-biphenyl-3-yl]-succinic acid;

2-[5-(5-carbamimidoyl-1H-benzoimidazol-2-yl)-3′-cyano-6-hydroxy-biphenyl-3-yl]-succinic acid;

2-[5-(5-carbamimidoyl-1H-benzoimidazol-2-yl)-5′-fluoro-6,2′-dihydroxy-biphenyl-3-yl]-succinic acid;

2-[5-(5-carbamimidoyl-1H-benzoimidazol-2-yl)-5′-chloro-6,2′-dihydroxy-biphenyl-3-yl]-succinic acid;

2-[5-(5-carbamimidoyl-1H-benzoimidazol-2-yl)-6-hydroxy-2′-hydroxymethyl-biphenyl-3-yl]-succinic acid;

2-[5-(5-carbamimidoyl-1H-indol-2-yl)-6,2′-dihydroxy-biphenyl-3-yl]-succinic acid;

2-[5-(5-carbamimidoyl-1H-benzoimidazol-2-yl)-5′-carboxy-6,2′-dihydroxy-biphenyl-3-yl]-succinic acid;

2-[5-(5-carbamimidoyl-1H-benzoimidazol-2-yl)-6,2′,5′-trihydroxy-biphenyl-3-yl]-succinic acid;

2-[5-(5-carbamimidoyl-1H-benzoimidazol-2-yl)-5′-cyano-6-hydroxy-2′-methoxy-biphenyl-3-yl]-succinic acid;

2-[5-(5-carbamimidoyl-1H-benzoimidazol-2-yl)-5′-aminocarbonyl-6-hydroxy-2′-methoxy-biphenyl-3-yl]-succinic acid;

2-[5-(5-carbamimidoyl-1H-benzoimidazol-2-yl)-6,2′,6′-trihydroxy-biphenyl-3-yl]-succinic acid;

2-[5-(5-carbamimidoyl-1H-benzoimidazol-2-yl)-6,2′-dihydroxy-5′-nitro-biphenyl-3-yl]-succinic acid;

2-[5-(5-carbamimidoyl-1H-benzoimidazol-2-yl)-2′-cyano-6-hydroxy-biphenyl-3-yl]-succinic acid;

2-[5-(6-carbamimidoyl-1H-benzoimidazol-2-yl)-6-hydroxy-3′-hydroxymethyl-biphenyl-3-yl]-succinic acid;

2-[5-(5-carbamimidoyl-1H-benzoimidazol-2-yl)-5′-cyano-6,2′-dihydroxy-biphenyl-3-yl]-succinic acid;

2-[5-(5-carbamimidoyl-1H-benzoimidazol-2-yl)-5′-aminocarbonyl-6,2′-dihydroxy-biphenyl-3-yl]-succinic acid;

2-[3′-bromo-5-(5-carbamimidoyl-1H-benzoimidazol-2-yl)-6,2′,6′-trihydroxy-biphenyl-3-yl]-succinic acid;

2-[5-(5-carbamimidoyl-1H-benzoimidazol-2-yl)-6,2′-dihydroxy-5′-methylsulfonylamino-biphenyl-3-yl]-succinic acid;

2-[5-(5-carbamimidoyl-1H-benzoimidazol-2-yl)-6,2′-dihydroxy-5′-isopropyl-biphenyl-3-yl]-succinic acid;

2-[5′-aminomethyl-5-(5-carbamimidoyl-1H-benzoimidazol-2-yl)-6,2′-dihydroxy-biphenyl-3-yl]-succinic acid;

2-[5-(5-carbamimidoyl-1H-benzoimidazol-2-yl)-6,2′-dihydroxy-5′-ureidomethyl-biphenyl-3-yl]-succinic acid;

2-[5-(5-carbamimidoyl-1H-benzoimidazol-2-yl)-6,2′-dihydroxy-5′-imidazol-2-yl-biphenyl-3-yl]-succinic acid;

2-[5′-amino-5-(5-carbamimidoyl-1H-benzoimidazol-2-yl)-6,2′-dihydroxy-biphenyl-3-yl]-succinic acid;

2-[5-(5-carbamimidoyl-1H-benzoimidazol-2-yl)-6,2′-dihydroxy-5′-ureido-biphenyl-3-yl]-succinic acid;

2-[5-(5-carbamimidoyl-1H-benzoimidazol-2-yl)-6,2′-dihydroxy-5′-(2-morpholin-4-ylethylaminocarbonyl-biphenyl-3-yl]-succinic acid;

2-[3′-bromo-5-(5-carbamimidoyl-1H-benzoimidazol-2-yl)-6,2′-dihydroxy-5′-cyanomethylbiphenyl-3-yl]-succinic acid;

2-[5-(5-carbamimidoyl-1H-benzoimidazol-2-yl)-5′-(2-cyanoethyl)-6,2′-dihydroxybiphenyl-3-yl]-succinic acid;

2-[3′bromo-5-(5-carbamimidoyl-1H-benzoimidazol-2-yl)-5′-carboxymethyl-6,2′-dihydroxy-biphenyl-3-yl]-succinic acid;

2-[5-(5-carbamimidoyl-1H-benzoimidazol-2-yl)-5′-(2-carboxyethyl)-6,2′-dihydroxy-biphenyl-3-yl]-succinic acid;

2-[2′-acetyl-5-(5-carbamimidoyl-1H-benzoimidazol-2-yl)-6-hydroxy-biphenyl-3-yl]-succinic acid;

2-[3′-bromo-5-(5-carbamimidoyl-1H-benzoimidazol-2-yl)-6,2′-dihydroxy-5′-aminocarbonylmethyl-biphenyl-3-yl]-succinic acid;

2-[5-(5-carbamimidoyl-1H-indol-2-yl)-5′-aminocarbonyl-6,2′-dihydroxy-biphenyl-3-yl]-succinic acid;

2-[5-(5-carbamimidoyl-1H-benzoimidazol-2-yl)-3′,5′-dichloro-6,2′-dihydroxy-biphenyl-3-yl]-succinic acid;

2-[5-(5-carbamimidoyl-1H-benzoimidazol-2-yl)-6,2′-dihydroxy-5′-[2-(2-hydroxyethoxy)ethylaminocarbonyl]-biphenyl-3-yl]-succinic acid;

2-[5-(5-carbamimidoyl-1H-benzoimidazol-2-yl)-4′,6′-dichloro-6,2′-dihydroxy-biphenyl-3-yl]-succinic acid;

2-[5-(5-carbamimidoyl-1H-benzoimidazol-2-yl)-6,2′-dihydroxy-5′-dimethylaminosulfonylamino-biphenyl-3-yl]-succinic acid;

2-[3′-bromo-5-(5-carbamimidoyl-1H-benzoimidazol-2-yl)-5′-chloro-6,2′-dihydroxy-biphenyl-3-yl]-succinic acid;

2-[5-(5-carbamimidoyl-1H-benzoimidazol-2-yl)-6,2′-dihydroxy-5′-(4-methyl-piperazin-1-ylcarbonyl)-biphenyl-3-yl]-succinic acid;

2-[5-(5-carbamimidoyl-1H-benzoimidazol-2-yl)-6,2′-dihydroxy-5′-(4-methyl-piperazin-1-ylmethyl)-biphenyl-3-yl]-succinic acid;

2-[5-(5-carbamimidoyl-1H-benzoimidazol-2-yl)-5′-carbamimidoyl-6,2′-dihydroxy-biphenyl-3-yl]-succinic acid;

2-[5-(5-carbamimidoyl-1H-benzoimidazol-2-yl)-5′-(2-dimethylaminoethylaminocarbonyl)-6,2′-dihydroxy-biphenyl-3-yl]-succinic acid;

2-[5-(5-carbamimidoyl-1H-benzoimidazol-2-yl)-6,2′-dihydroxy-5′-hydroxymethyl-biphenyl-3-yl]-succinic acid;

2-[5-(5-carbamimidoyl-1H-benzoimidazol-2-yl)-6,2′-dihydroxy-5′-methylureidomethylbiphenyl-3-yl]-succinic acid;

2-[3′-aminosulfonyl-5-(5-carbamimidoyl-1H-benzoimidazol-2-yl)-6-hydroxy-biphenyl-3-yl]-succinic acid;

2-[5-(5-carbamimidoyl-1H-benzoimidazol-2-yl)-5′-dimethylaminosulfonyl-amino-6,2′-dihydroxy-biphenyl-3-yl]-succinic acid;

2-[5-(5-carbamimidoyl-6-fluoro-1H-benzoimidazol-2-yl)-6,2′-dihydroxy-biphenyl-3-yl]-succinic acid;

2-[5-(5-carbamimidoyl-6-chloro-1H-indol-2-yl)-6,2′-dihydroxy-biphenyl-3-yl]-succinic acid;

diethyl 2-[5-(5-carbamimidoyl-1H-benzoimidazol-2-yl)-5′-fluoro-6,2′-dihydroxy-biphenyl-3-yl]-succinate;

2-[5-(5-carbamimidoyl-5-fluoro-1H-benzoimidazol-2-yl)-5′-fluoro-6,2′-dihydroxy-biphenyl-3-yl]-succinic acid;

2-[5-(5-carbamimidoyl-1H-benzoimidazol-2-yl)-5′-fluoro-6,2′-dihydroxy-biphenyl-3-yl]-2-methylsuccinic acid;

2-[5-(5-carbamimidoyl-1H-benzoimidazol-2-yl)-5′-fluoro-6,2′-dihydroxy-biphenyl-3-yl]-succinic acid 1-ethyl ester;

2-[5-(5-carbamimidoyl-1H-benzoimidazol-2-yl)-5′-fluoro-6,2′-dihydroxy-biphenyl-3-yl]-succinic acid 4-ethyl ester;

(Z)-2-[5-(5-carbamimidoyl-1H-benzoimidazol-2-yl)-5′-fluoro-6-hydroxy-2′methoxy-biphenyl-3-yl]-but-2-enedioic acid;

(Z)-2-[5-(5-carbamimidoyl-1H-benzoimidazol-2-yl)-5′-fluoro-6,2′-dihydroxy-biphenyl-3-yl]-but-2-enedioic acid;

(E)-2-[5-(5-carbamimidoyl-1H-benzoimidazol-2-yl)-5′-fluoro-6,2′-dihydroxy-biphenyl-3-yl]-but-2-enedioic acid;

3-[5-(5-carbamimidoyl-1H-benzoimidazol-2-yl)-5′-fluoro-6,2′-dihydroxy-biphenyl-3-yl]-propionic acid;

methyl 3-[5-(5-carbamimidoyl-1H-benzoimidazol-2-yl)-5′-fluoro-6,2′-dihydroxy-biphenyl-3-yl]-propionate;

methyl 2-[5-(5-carbamimidoyl-1H-benzoimidazol-2-yl)-5′-fluoro-6,2′-dihydroxy-biphenyl-3-yl]-acetate;

2-[5-(5-carbamimidoyl-1H-benzoimidazol-2-yl)-6,2′-dihydroxy-biphenyl-3-yl]-acetic acid;

2-[5-(5-carbamimidoyl-1H-benzoimidazol-2-yl)-5′-fluoro-6,2′-dihydroxy-biphenyl-3-yl]-acetic acid;

2-[5-(5-N-hydroxycarbamimidoyl-1H-benzoimidazol-2-yl)-5′-fluoro-6,2′-dihydroxy-biphenyl-3-yl]-succinic acid;

diethyl 2-[5-(5-N-hydroxycarbamimidoyl-1H-benzoimidazol-2-yl)-5′-fluoro-6,2′-dihydroxy-biphenyl-3-yl]-succinate;

2-[5-(5-N-hydroxycarbamimidoyl-1H-benzoimidazol-2-yl)-6,2′-dihydroxy-5′-methylsulfonylaminobiphenyl-3-yl]-succinic acid;

diethyl 2-[5-(5-N-hydroxycarbamimidoyl-1H-benzoimidazol-2-yl)-6,2′-dihydroxy-5′-ureidomethylbiphenyl-3-yl]-succinate;

2-[5-(5-N-hydroxycarbamimidoyl-1H-benzoimidazol-2-yl)-5′-fluoro-6,2′-dihydroxy-biphenyl-3-yl]-acetic acid;

diethyl 2-[5-(5-N-hydroxycarbamimidoyl-1H-benzoimidazol-2-yl)-6,2′-dihydroxy-5′-hydroxymethylbiphenyl-3-yl]-succinate;

dimethyl 2-[5-(5-N-hydroxycarbamimidoyl-1H-benzoimidazol-2-yl)-5′-aminocarbonyl-6,2′-dihydroxybiphenyl-3-yl]-succinate; and

2-[5-(5-N-hydroxycarbamimidoyl-1H-benzoimidazol-2-yl)-6,2′-dihydroxy-5′-ureidomethylbiphenyl-3-yl]-succinic acid.

The compounds of Formula I and the intermediates and starting materials used in their preparation are named generally by AutoNom 4.0 (Beilstein Information Systems, Inc.).

### Preferred Embodiments

While the broadest definition of this invention is set forth in the Summary of the Invention, certain compounds of Formula I are preferred. For example:

I.

(a) One preferred group of compounds is that wherein X¹ is —N— and X², X³, and X⁴ are —CR⁵— where R⁵ is hydrogen.

(b) Another preferred group of compounds is that wherein X¹ is —N—; X² and X⁴ are —CR⁵— where R⁵ is hydrogen and X³ is —CR⁵— where R⁵ is halo, preferably fluoro or chloro.

(c) Yet another preferred group of compounds is that wherein X¹ is —CH— and X², X³, and X⁴ are —CR⁵— where R⁵ is hydrogen.

(d) Another preferred group of compounds is that wherein X¹ is —CH—; X² and X⁴ are —CR⁵— where R⁵ is hydrogen and X³ is —CR⁵— where R⁵ is halo, preferably fluoro or chloro.

Within the above preferred groups (a-d), a more preferred group of compounds is that wherein R¹, R² and R¹³ are hydrogen; R³ is a group of formula (a) wherein n is 0; R⁸ and R¹⁰ are hydrogen; and R⁹ and R¹¹ are independently hydrogen or alkyl, preferably hydrogen, methyl, or ethyl. More preferably one of R⁹ and R¹¹ is hydrogen and the other of R⁹ and R¹¹ is ethyl.

Another more preferred group of compounds is that wherein R¹ and R² are hydrogen; R¹³ is hydroxy; and R³ is a group of formula (a) wherein n is 0; R⁸ and R¹⁰ are hydrogen; and R⁹ and R¹¹ are independently hydrogen or alkyl, preferably hydrogen, methyl, or ethyl. More preferably one of R⁹ and R¹¹ is hydrogen and the other of R⁹ and R¹¹ is ethyl.

Another more preferred group of compounds is that wherein R¹ and R² are hydrogen; R¹³ is hydrogen; R³ is a group of formula (a) wherein n is 0; R⁸ and R¹⁰ are hydrogen; and R⁹ and R¹¹ are aryl, aralkyl, or haloalkyl, preferably phenyl, benzyl or —CH₂CCl₃.

Another more preferred group of compounds is that wherein R¹ and R² are hydrogen; R¹³ is hydroxy; and R³ is a group of formula (a) wherein n is 0; R⁸ and R¹⁰ are hydrogen; and R⁹ and R¹¹ are aryl, aralkyl, or haloalkyl, preferably phenyl, benzyl or —CH₂CCl₃.

Another more preferred group of compounds is that wherein R¹, R² and R¹³ are hydrogen; R³ is a group of formula (a) wherein n is 0; R⁸, R⁹, R¹⁰ and R¹¹ are hydrogen.

Another more preferred group of compounds is that wherein R¹ and R² are hydrogen; R³ is a group of formula (a) wherein n is 0; R⁸, R⁹, R¹⁰ and R¹¹ are hydrogen; and R¹³ is hydroxy.

Yet another more preferred group of compounds of Formula I is that wherein R¹, R² and R¹³ hydrogen; R³ is a group of formula (a) wherein n is 0; R⁸ and R¹⁰ together from a covalent bond; and R⁹ and R¹¹ are independently hydrogen, methyl or ethyl, preferably hydrogen.

Yet another more preferred group of compounds is that wherein R¹, R² and R¹³ are hydrogen; R³ is a -(alkylene)-COOR⁹ where R⁹ is hydrogen or alkyl. Preferably R³ is —CH₂COOR⁹, —(CH₂)₂COOR⁹ wherein R⁹ is hydrogen, methyl or ethyl.

Yet another more preferred group of compounds is that wherein R¹ and R² are hydrogen; R¹³ is hydroxy, and R³ is a -(alkylene)-COOR⁹ where R⁹ is hydrogen or alkyl. Preferably R³ is —CH₂COOR⁹, —(CH₂)₂COOR⁹ wherein R⁹ is hydrogen, methyl or ethyl, preferably hydrogen or ethyl.

Yet another more preferred group of compounds is that wherein R¹, R² and R¹³ are hydrogen; R³ is a -(alkylene)-COOR⁹ where R⁹ is aryl, aralkyl, or haloalkyl. Preferably R³ is —CH₂COOR⁹, —(CH₂)₂COOR⁹ wherein R⁹ is phenyl, benzyl or —CH₂CCl₃.

Yet another more preferred group of compounds is that wherein R¹ and R² are hydrogen; R¹³ is hydroxy, and R³ is a -(alkylene)-COOR⁹ where R⁹ is aryl, aralkyl, or haloalkyl. Preferably R³ is —CH₂COOR⁹, —(CH₂)₂COOR⁹ wherein R⁹ is phenyl, benzyl or —CH₂CCl₃.

Within the above preferred and more preferred groups, an even more preferred group of compounds is:

(i) R⁴ is hydroxy, hydroxymethyl, or 2-hydroxyethyl and is located at the 2′-position of the biphenyl ring, preferably R⁴ is hydroxy or hydroxymethyl, more preferably hydroxy; and R⁶ and R⁷ are hydrogen; or

(ii) R⁴ is hydroxy and is located at the 2′-position of the biphenyl ring.

Within this group (ii), a more preferred group of compounds is that wherein R⁶ is hydrogen and R⁷ is located at the 5′-position of the biphenyl ring and is alkyl, halo, hydroxy, nitro, cyano, alkoxy, alkylthio, haloalkyl, haloalkoxy, cyanoalkyl, alkoxyalkyl, hydroxyalkyl, carboxy, alkoxycarbonyl, carboxyalkyl, alkoxycarbonylalkyl, amino, alkylamino, dialkylamino, aminoalkyl, acylamino, aminosulfonylamino, alkylaminosulfonylamino, alkylsulfonyl, arylsufonyl, heteroarylsulfonyl, heteroaralkyl, heterocycloalkylalkylaminocarbonyl, hydroxyalkoxyalkylaminocarbonyl, heterocycloalkylcarbonyl, heterocycloalkylalkyl, oxoheterocycloalkylalkyl, carbamimidoyl, hydroxycarbamimidoyl, alkoxycarbamimidoyl, aminosulfonyl, aminosulfonylalkyl, alkylsulfonylamino, alkoxysulfonylamino, heteroaryl, ureido, alkylureido, dialkylureido, ureidoalkyl, alkylureidoalkyl, dialkylureidoalkyl, thioureido, thioureidoalkyl, —COR¹² (where R¹² is alkyl or haloalkyl), —(CH₂)—COR¹² (where R¹² is alkyl or haloalkyl), aminocarbonyl, —CONR¹⁴R¹⁵ (where R¹⁴ is hydrogen or alkyl and R¹⁵ is alkyl), aminocarbonylalkyl, —(CH₂)—CONR¹⁶R¹⁷ (where R¹⁶ is hydrogen or alkyl and R¹⁷ is alkyl), —(CH₂)—NR²⁰R²¹ (where R²⁰ is hydrogen or alkyl and R²¹ is alkyl), —SO₂NR²²R²³ (where R²² is hydrogen or alkyl and R²³ is alkyl), —(CH₂)—SO₂NR²⁴R²⁵ (where R²⁴ is hydrogen or alkyl and R²⁵ is alkyl), —NHSO₂NR²⁷R²⁸ (where R²⁷ is hydrogen or alkyl, and R²⁸ is alkyl), —(CH₂)—NHSO₂NR³⁰R³¹ (where R³⁰ is hydrogen or alkyl, and R³¹ is hydrogen or alkyl).

Within this group (ii), another more preferred group of compounds is that wherein R⁷ is alkyl, halo, hydroxy, hydroxyalkyl, carboxy, alkoxy, cyano, nitro, amino, aminocarbonyl, -alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

Within this group (ii), yet another more preferred group of compounds is that wherein R⁷ is alkyl, halo, hydroxy, hydroxyalkyl, carboxy, alkoxy, cyano, nitro, aminocarbonyl, alkylsulfonylamino, aminoalkyl, ureidoalkyl, ureido, cyanoalkyl, carboxyalkyl, aminocarbonylalkyl, or heteroaryl, preferably methyl, isopropyl, chloro, fluoro, hydroxymethyl, carboxy, methoxy, cyano, nitro, aminocarbonyl, methylsulfonylamino, aminomethyl, ureidomethyl, imidazol-2-yl, amino, ureido, 2-cyanoethyl, carboxymethyl, 2-carboxyethyl, aminocarbonylmethyl, or dimethylaminosulfonylamino.

Within this group (ii), yet another more preferred group of compounds is that wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, acetyl, or aminosulfonyl. Even more preferably R⁷ is ureidomethyl, aminocarbonyl, aminosulfonyl, or fluoro; or

(iii) R⁴ is hydroxymethyl and is located at the 2′-position of the biphenyl ring.

Within this group (iii), a more preferred group of compounds is that wherein R⁶ is hydrogen and R⁷ is located at the 5′-position of the biphenyl ring and is alkyl, halo, hydroxy, nitro, cyano, alkoxy, alkylthio, haloalkyl, haloalkoxy, cyanoalkyl, alkoxyalkyl, hydroxyalkyl, carboxy, alkoxycarbonyl, carboxyalkyl, alkoxycarbonylalkyl, amino, alkylamino, dialkylamino, aminoalkyl, acylamino, aminosulfonylamino, alkylaminosulfonylamino, alkylsulfonyl, arylsufonyl, heteroarylsulfonyl, heteroaralkyl, heterocycloalkylalkylaminocarbonyl, hydroxyalkoxyalkylaminocarbonyl, heterocycloalkylcarbonyl, heterocycloalkylalkyl, oxoheterocycloalkylalkyl, carbamimidoyl, hydroxycarbamimidoyl, alkoxycarbamimidoyl, aminosulfonyl, aminosulfonylalkyl, alkylsulfonylamino, alkoxysulfonylamino, heteroaryl, ureido, alkylureido, dialkylureido, ureidoalkyl, alkylureidoalkyl, dialkylureidoalkyl, thioureido, thioureidoalkyl, —COR¹² (where R¹² is alkyl or haloalkyl), —(CH₂)—COR¹² (where R¹² is alkyl or haloalkyl), aminocarbonyl, —CONR¹³R¹⁵ (where R¹⁴ is hydrogen or alkyl and R¹⁵ is alkyl), aminocarbonylalkyl, —(CH₂)—CONR¹⁶R¹⁷ (where R¹⁶ is hydrogen or alkyl and R¹⁷ is alkyl), —(CH₂)—NR²⁰R²¹ (where R²⁰ is hydrogen or alkyl and R²¹ is alkyl), —SO₂NR²²R²³ (where R²² is hydrogen or alkyl and R²³ is alkyl), —(CH₂)—SO₂NR²⁴R²⁵ (where R²⁴ is hydrogen or alkyl and R²⁵ is alkyl), —NHSO₂NR²⁷R²⁸ (where R²⁷ is hydrogen or alkyl, and R²⁸ is alkyl), —(CH₂)—NHSO₂NR³⁰R³¹ (where R³⁰ is hydrogen or alkyl, and R³¹ is hydrogen or alkyl).

Within this group (iii), another more preferred group of compounds is that wherein R⁷ is alkyl, halo, hydroxy, hydroxyalkyl, carboxy, alkoxy, cyano, nitro, amino, aminocarbonyl, -alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

Within this group (iii), yet another more preferred group of compounds is that wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, acetyl, or aminosulfonyl. Even more preferably R⁷ is ureidomethyl, aminocarbonyl, aminosulfonyl, or fluoro; or

(iv) R⁴ is aminosulfonyl, methylaminosulfonyl, or dimethylaminosulfonyl, preferably aminosulfonyl and is located at the 2′-position of the biphenyl ring.

Within this group (iv), a more preferred group of compounds is that wherein R⁶ is hydrogen and R⁷ is located at the 5′-position of the biphenyl ring and is alkyl, halo, hydroxy, nitro, cyano, alkoxy, alkylthio, haloalkyl, haloalkoxy, cyanoalkyl, alkoxyalkyl, hydroxyalkyl, carboxy, alkoxycarbonyl, carboxyalkyl, alkoxycarbonylalkyl, amino, alkylamino, dialkylamino, aminoalkyl, acylamino, aminosulfonylamino, alkylaminosulfonylamino, alkylsulfonyl, arylsufonyl, heteroarylsulfonyl, heteroaralkyl, heterocycloalkylalkylaminocarbonyl, hydroxyalkoxyalkylaminocarbonyl, heterocycloalkylcarbonyl, heterocycloalkylalkyl, oxoheterocycloalkylalkyl, carbamimidoyl, hydroxycarbamimidoyl, alkoxycarbamimidoyl, aminosulfonyl, aminosulfonylalkyl, alkylsulfonylamino, alkoxysulfonylamino, heteroaryl, ureido, alkylureido, dialkylureido, ureidoalkyl, alkylureidoalkyl, dialkylureidoalkyl, thioureido, thioureidoalkyl, —COR¹² (where R¹ is alkyl or haloalkyl), —(CH₂)—COR¹² (where R¹² is alkyl or haloalkyl), aminocarbonyl, —CONR¹⁴R¹⁵ (where R¹⁴ is hydrogen or alkyl and R¹⁵ is alkyl), aminocarbonylalkyl, —(CH₂)—CONR¹⁶R¹⁷ (where R¹⁶ is hydrogen or alkyl and R¹⁷ is alkyl), —(CH₂)—NR²⁰R²¹ (where R²⁰ is hydrogen or alkyl and R²¹ is alkyl), —SO₂NR²²R²³ (where R²² is hydrogen or alkyl and R²³ is alkyl), —(CH₂)—SO₂NR²⁴R²⁵ (where R²⁴ is hydrogen or alkyl and R²⁵ is alkyl), —NHSO₂NR²⁷R²⁸ (where R²⁷ is hydrogen or alkyl, and R²⁸ is alkyl), —(CH₂)—NHSO₂NR³⁰R³¹ (where R³⁰ is hydrogen or alkyl, and R³¹ is hydrogen or alkyl).

Within this group (iv), another more preferred group of compounds is that wherein R⁷ is alkyl, halo, hydroxy, hydroxyalkyl, carboxy, alkoxy, cyano, nitro, amino, aminocarbonyl, -alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

Within this group (iv), yet another more preferred group of compounds is that wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, acetyl, or aminosulfonyl. Even more preferably R⁷ is ureidomethyl, aminocarbonyl, aminosulfonyl, or fluoro; or

(v) R⁴ is hydroxy and is located at the 2′-position of the biphenyl ring; R⁶ is hydrogen; and R⁷ is located at the 6′-position of the biphenyl ring. Preferably R⁷ is alkyl, halo, hydroxy, hydroxyalkyl, carboxy, alkoxy, cyano, nitro, aminocarbonyl, alkylsulfonylamino, aminoalkyl, ureidoalkyl, ureido, cyanoalkyl, carboxyalkyl, aminocarbonylalkyl, heteroaryl, preferably methyl, isopropyl, chloro, fluoro, hydroxymethyl, 2-hydroxyethyl, carboxy, methoxy, cyano, nitro, aminocarbonyl, methylsulfonylamino, aminomethyl, ureidomethyl, imidazolyl, amino, ureido, 2-cyanoethyl, carboxymethyl, 2-carboxyethyl, aminocarbonylmethyl, or dimethylaminosulfonylamino. Even more preferably R⁷ is hydroxy; or

(vi) R⁴ and R⁵ are hydrogen and R⁷ is located at the 3′-position of the biphenyl ring. Preferably, R⁷ is aminosulfonyl, haloalkoxy, hydroxy, hydroxyalkyl, aminocarbonyl, ureidoalkyl, cyanoalkyl, alkoxyalkyl, carboxyalkyl, aminocarbonylalkyl, heterocycloalkylalkyl, —COR¹² (where R¹² is alkyl) or cyano, more preferably aminosulfonyl, difluoromethoxy, hydroxy, hydroxymethyl, 2-hydroxyethyl, ureidomethyl, or aminocarbonyl. Most preferably, R⁷ is aminosulfonyl.

II.

Yet another preferred group of compounds is that wherein:

(i) R⁴ is hydroxy, hydroxymethyl, or 2-hydroxyethyl and is located at the 2′-position of the biphenyl ring, preferably R⁴ is hydroxy or hydroxymethyl, more preferably hydroxy; and R⁶ and R⁷ are hydrogen; or

(ii) R⁴ is hydroxy and is located at the 2′-position of the biphenyl ring.

Within this group (ii), a more preferred group of compounds is that wherein R⁶ is hydrogen and R⁷ is located at the 5′-position of the biphenyl ring and is alkyl, halo, hydroxy, nitro, cyano, alkoxy, alkylthio, haloalkyl, haloalkoxy, cyanoalkyl, alkoxyalkyl, hydroxyalkyl, carboxy, alkoxycarbonyl, carboxyalkyl, alkoxycarbonylalkyl, amino, alkylamino, dialkylamino, aminoalkyl, acylamino, aminosulfonylamino, alkylaminosulfonylamino, alkylsulfonyl, arylsufonyl, heteroarylsulfonyl, heteroaralkyl, heterocycloalkylalkylaminocarbonyl, hydroxyalkoxyalkylaminocarbonyl, heterocycloalkylcarbonyl, heterocycloalkylalkyl, oxoheterocycloalkylalkyl, carbamimidoyl, hydroxycarbamimidoyl, alkoxycarbamimidoyl, aminosulfonyl, aminosulfonylalkyl, alkylsulfonylamino, alkoxysulfonylamino, heteroaryl, ureido, alkylureido, dialkylureido, ureidoalkyl, alkylureidoalkyl, dialkylureidoalkyl, thioureido, thioureidoalkyl, —COR¹² (where R¹² is alkyl or haloalkyl), —(CH₂)—COR¹² (where R¹² is alkyl or haloalkyl), aminocarbonyl, —CONR¹⁴R¹⁵ (where R¹⁴ is hydrogen or alkyl and R¹⁵ is alkyl), aminocarbonylalkyl, —(CH₂)—CONR¹⁶R¹⁷ (where R¹⁶ is hydrogen or alkyl and R¹⁷ is alkyl), —(CH₂)—NR²⁰R²¹ (where R²⁰ is hydrogen or alkyl and R²¹ is alkyl), —SO₂NR²²R²³ (where R²² is hydrogen or alkyl and R²³ is alkyl), —(CH₂)—SO₂NR²⁴R²⁵ (where R²⁴ is hydrogen or alkyl and R²⁵ is alkyl), —NHSO₂NR²⁷R²⁸ (where R²⁷ is hydrogen or alkyl, and R²⁸ is alkyl), —(CH₂)—NHSO₂NR³⁰R³¹ (where R³⁰ is hydrogen or alkyl, and R³¹ is hydrogen or alkyl).

Within this group (ii), another more preferred group of compounds is that wherein R⁷ is alkyl, halo, hydroxy, hydroxyalkyl, carboxy, alkoxy, cyano, nitro, amino, aminocarbonyl, -alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

Within this group (ii), yet another more preferred group of compounds is that wherein R⁷ is alkyl, halo, hydroxy, hydroxyalkyl, carboxy, alkoxy, cyano, nitro, aminocarbonyl, alkylsulfonylamino, aminoalkyl, ureidoalkyl, ureido, cyanoalkyl, carboxyalkyl, aminocarbonylalkyl, or heteroaryl, preferably methyl, isopropyl, chloro, fluoro, hydroxymethyl, carboxy, methoxy, cyano, nitro, aminocarbonyl, methylsulfonylamino, aminomethyl, ureidomethyl, imidazol-2-yl, amino, ureido, 2-cyanoethyl, carboxymethyl, 2-carboxyethyl, aminocarbonylmethyl, or dimethylaminosulfonylamino.

Within this group (ii), yet another more preferred group of compounds is that wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, acetyl, or aminosulfonyl. Even more preferably R⁷ is ureidomethyl, aminocarbonyl, aminosulfonyl, or fluoro; or

(iii) R⁴ is hydroxymethyl and is located at the 2′-position of the biphenyl ring.

Within this group (iii), a more preferred group of compounds is that wherein R⁶ is hydrogen and R⁷ is located at the 5′-position of the biphenyl ring and is alkyl, halo, hydroxy, nitro, cyano, alkoxy, alkylthio, haloalkyl, haloalkoxy, cyanoalkyl, alkoxyalkyl, hydroxyalkyl, carboxy, alkoxycarbonyl, carboxyalkyl, alkoxycarbonylalkyl, amino, alkylamino, dialkylamino, aminoalkyl, acylamino, aminosulfonylamino, alkylaminosulfonylamino, alkylsulfonyl, arylsufonyl, heteroarylsulfonyl, heteroaralkyl, heterocycloalkylalkylaminocarbonyl, hydroxyalkoxyalkylaminocarbonyl, heterocycloalkylcarbonyl, heterocycloalkylalkyl, oxoheterocycloalkylalkyl, carbamimidoyl, hydroxycarbamimidoyl, alkoxycarbamimidoyl, aminosulfonyl, aminosulfonylalkyl, alkylsulfonylamino, alkoxysulfonylamino, heteroaryl, ureido, alkylureido, dialkylureido, ureidoalkyl, alkylureidoalkyl, dialkylureidoalkyl, thioureido, thioureidoalkyl, —COR¹² (where R¹² is alkyl or haloalkyl), —(CH₂)—COR¹² (where R¹² is alkyl or haloalkyl), aminocarbonyl, —CONR¹⁴R¹⁵ (where R¹⁴ is hydrogen or alkyl and R¹⁵ is alkyl), aminocarbonylalkyl, —(CH₂)—CONR¹⁶R¹⁷ (where R¹⁶ is hydrogen or alkyl and R¹⁷ is alkyl), —(CH₂)—NR²⁰R²¹ (where R²⁰ is hydrogen or alkyl and R²¹ is alkyl), —SO₂NR²²R²³ (where R²² is hydrogen or alkyl and R²³ is alkyl), —(CH₂)—SO₂NR²⁴R²⁵ (where R²⁴ is hydrogen or alkyl and R²⁵ is alkyl), —NHSO₂NR²⁷R²⁸ (where R²⁷ is hydrogen or alkyl, and R²⁸ is alkyl), —(CH₂)—NHSO₂NR³⁰R³¹ (where R³⁰ is hydrogen or alkyl, and R³¹ is hydrogen or alkyl).

Within this group (iii), another more preferred group of compounds is that wherein R⁷ is alkyl, halo, hydroxy, hydroxyalkyl, carboxy, alkoxy, cyano, nitro, amino, aminocarbonyl, -alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

Within this group (iii), yet another more preferred group of compounds is that wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, acetyl, or aminosulfonyl. Even more preferably R⁷ is ureidomethyl, aminocarbonyl, aminosulfonyl, or fluoro; or

(iv) R⁴ is aminosulfonyl, methylaminosulfonyl, or dimethylaminosulfonyl, preferably aminosulfonyl and is located at the 2′-position of the biphenyl ring.

Within this group (iv), a more preferred group of compounds is that wherein R⁶ is hydrogen and R⁷ is located at the 5′-position of the biphenyl ring and is alkyl, halo, hydroxy, nitro, cyano, alkoxy, alkylthio, haloalkyl, haloalkoxy, cyanoalkyl, alkoxyalkyl, hydroxyalkyl, carboxy, alkoxycarbonyl, carboxyalkyl, alkoxycarbonylalkyl, amino, alkylamino, dialkylamino, aminoalkyl, acylamino, aminosulfonylamino, alkylaminosulfonylamino, alkylsulfonyl, arylsufonyl, heteroarylsulfonyl, heteroaralkyl, heterocycloalkylalkylaminocarbonyl, hydroxyalkoxyalkylaminocarbonyl, heterocycloalkylcarbonyl, heterocycloalkylalkyl, oxoheterocycloalkylalkyl, carbamimidoyl, hydroxycarbamimidoyl, alkoxycarbamimidoyl, aminosulfonyl, aminosulfonylalkyl, alkylsulfonylamino, alkoxysulfonylamino, heteroaryl, ureido, alkylureido, dialkylureido, ureidoalkyl, alkylureidoalkyl, dialkylureidoalkyl, thioureido, thioureidoalkyl, —COR¹² (where R¹² is alkyl or haloalkyl), —(CH₂)—COR¹² (where R¹² is alkyl or haloalkyl), aminocarbonyl, —CONR¹⁴R¹⁵ (where R¹⁴ is hydrogen or alkyl and R¹⁵ is alkyl), aminocarbonylalkyl, —(CH₂)—CONR¹⁶R¹⁷ (where R¹⁶ is hydrogen or alkyl and R¹⁷ is alkyl), —(CH₂)—NR²⁰R²¹ (where R²⁰ is hydrogen or alkyl and R²¹ is alkyl), —SO₂NR²²R²³ (where R²² is hydrogen or alkyl and R²³ is alkyl), —(CH₂)—SO₂NR²⁴R²⁵ (where R²⁴ is hydrogen or alkyl and R²⁵ is alkyl), —NHSO₂NR²⁷R²⁸ (where R²⁷ is hydrogen or alkyl, and R²⁸ is alkyl), —(CH₂)—NHSO₂NR³⁰ R³¹ (where R³⁰ is hydrogen or alkyl, and R³¹ is hydrogen or alkyl).

Within this group (iv), another more preferred group of compounds is that wherein R⁷ is alkyl, halo, hydroxy, hydroxyalkyl, carboxy, alkoxy, cyano, nitro, amino, aminocarbonyl, -alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

Within this group (iv), yet another more preferred group of compounds is that wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, acetyl, or aminosulfonyl. Even more preferably R⁷ is ureidomethyl, aminocarbonyl, aminosulfonyl, or fluoro; or

(v) R⁴ is hydroxy and is located at the 2′-position of the biphenyl ring; R⁶ is hydrogen; and R⁷ is located at the 6′-position of the biphenyl ring. Preferably R⁷ is alkyl, halo, hydroxy, hydroxyalkyl, carboxy, alkoxy, cyano, nitro, aminocarbonyl, alkylsulfonylamino, aminoalkyl, ureidoalkyl, ureido, cyanoalkyl, carboxyalkyl, aminocarbonylalkyl, heteroaryl, preferably methyl, isopropyl, chloro, fluoro, hydroxymethyl, 2-hydroxyethyl, carboxy, methoxy, cyano, nitro, aminocarbonyl, methylsulfonylamino, aminomethyl, ureidomethyl, imidazolyl, amino, ureido, 2-cyanoethyl, carboxymethyl, 2-carboxyethyl, aminocarbonylmethyl, or dimethylaminosulfonylamino. Even more preferably R⁷ is hydroxy; or

(vi) R⁴ and R⁵ are hydrogen and R⁷ is located at the 3′-position of the biphenyl ring. Preferably, R⁷ is aminosulfonyl, haloalkoxy, hydroxy, hydroxyalkyl, aminocarbonyl, ureidoalkyl, cyanocarbonyl, alkoxyalkyl, carboxyalkyl, aminocarbonylalkyl, heterocycloalkylalkyl, —COR¹² (where R¹² is alkyl) or cyano, more preferably aminosulfonyl, difluoromethoxy, hydroxy, hydroxymethyl, 2-hydroxyethyl, ureidomethyl, or aminocarbonyl. Most preferably, R⁷ is aminosulfonyl.

With the above preferred group II, a more preferred group of compounds is that wherein:

X¹ is —N— and X², X³, and X⁴ are —CR⁵— where R⁵ is hydrogen and R¹³ is hydrogen; or

X¹ is —N—; X² and X⁴ are —CR⁵— where R⁵ is hydrogen and X³ is —CR⁵— where R⁵ is halo, preferably fluoro or chloro, and R¹³ is hydrogen; or

X¹ is —CH— and X², X³, and X⁴ are —CR⁵— where R⁵ is hydrogen, and R¹³ is hydrogen; or

X is —CH—; X² and X⁴ are —CR⁵— where R⁵ is hydrogen and X³ is —CR⁵— where R⁵ is halo, preferably fluoro or chloro, and R¹³ is hydrogen.

III.

Yet another preferred group of compounds of Formula I are those wherein the moiety:

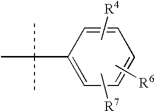

is 2′-acetylphenyl, 3′-acetylphenyl, 3′-hydroxyphenyl, 2′-hydroxyphenyl, 3′-aminocarbonylphenyl, 3′-cyanophenyl, 5′-fluoro-2′-hydroxyphenyl, 5′-chloro-2′-hydroxyphenyl, 2′-hydroxy-methylphenyl, 2′-hydroxyphenyl, 5′-carboxy-2′-hydroxyphenyl, 2′,5′-dihydroxyphenyl, 5′-cyano-2′-methoxyphenyl, 5′-aminocarbonyl-2′-methoxyphenyl, 2′,6′-dihydroxyphenyl, 2′-hydroxy-5′-nitrophenyl, 2′-cyanophenyl, 3′-hydroxymethylphenyl, 3′-(2-hydroxyethyl)phenyl, 5′-cyano-2′-hydroxyphenyl, 5′-aminocarbonyl-2′-hydroxyphenyl, 3′-bromo-2′,6′-dihydroxyphenyl, 5′-aminomethyl-2′-hydroxyphenyl, 2′-hydroxy-5′-ureidomethylphenyl, 2′-hydroxy-5′-imidazol-2-ylphenyl, 5′-amino-2′-hydroxyphenyl, 2′-hydroxy-5′-ureidophenyl, 2′-hydroxy-5′-(2-morpholin-4-ylethyl)-aminocarbonylphenyl, 3′-bromo-2′-hydroxy-5′-cyanomethylphenyl, 5′-(2-cyanoethyl)-2′-hydroxyphenyl, 3′-bromo-5′-carboxymethyl-2′-hydroxyphenyl, 5′-(2-carboxyethyl)-2′-hydroxyphenyl, 5′-aminocarbonylmethyl-2′-hydroxyphenyl, 3′,5′-dichloro-2′-hydroxyphenyl, 2′-hydroxy-5′-[2-(2-hydroxyethoxy)ethylaminocarbonyl]phenyl, 5′-dimethylaminosulfonyl-amino-2′-hydroxy-phenyl, 3′-bromo-5′-chloro-2′-hydroxyphenyl, 2′-hydroxy-5′-(4-methyl-piperazin-1-ylcarbonyl)phenyl, 2′-hydroxy-5′-(4-methylpiperazin-1-ylmethyl)phenyl, 5′-amidino-2′-hydroxyphenyl, 5′-(2-dimethylaminoethylaminocarbonyl)-2′-hydroxyphenyl, 3′-aminosulfonylphenyl, 2′-hydroxy-5′-aminosulfonylphenyl, 2′-hydroxy-5′-hydroxymethyl-phenyl, 2′-hydroxy-5′-(2-hydroxyethyl)phenyl, 2′-hydroxy-5′-dimethylaminosulfonylaminophenyl, 5′-aminocarbonyl-2′-hydroxyphenyl, or 2′-hydroxy-5′-(CH₃NHCONHCH₂)phenyl. Preferably 2′-hydroxyphenyl, 5′-fluoro-2′-hydroxyphenyl, 5′-chloro-2′-hydroxyphenyl, 2′-hydroxymethylphenyl, 2′-(2-hydroxyethyl)phenyl, 2′-hydroxy-phenyl, 5′-carboxy-2′-hydroxyphenyl, 2′,5′-dihydroxyphenyl, 2′,6′-dihydroxyphenyl, 2′-hydroxy-5′-nitro-phenyl, 5′-cyano-2′-hydroxyphenyl, 5′-aminocarbonyl-2′-hydroxyphenyl, 2′,6′-dihydroxy-phenyl, 5′-aminomethyl-2′-hydroxyphenyl, 2′-hydroxy-5′-ureidomethylphenyl, 2′-hydroxy-5′-imidazol-2-ylphenyl, 5′-amino-2′-hydroxyphenyl, 2′-hydroxy-5′-ureidophenyl, 2′-hydroxy-5′-(2-morpholin-4-ylethyl)aminocarbonyl-phenyl, 3′-bromo-2′-hydroxy-5′-hydroxymethylphenyl, 5′-(2-cyanoethyl)-2′-hydroxyphenyl, 3′-bromo-5′-carboxymethyl-2′-hydroxyphenyl, 5′-(2-carboxyethyl)-2′-hydroxyphenyl, 5′-aminocarbonylmethyl-2′-hydroxyphenyl, 3′,5′-dichloro-2′-hydroxyphenyl, 2′-hydroxy-5′-[2-(2-hydroxyethoxy)ethylaminocarbonyl]phenyl, 5′-dimethyl-aminosulfonylamino-2′-hydroxy-phenyl, 3′-bromo-5′-chloro-2′-hydroxyphenyl, 2′-hydroxy-5′-(4-methylpiperazin-1-ylcarbonyl)phenyl, 2′-hydroxy-5′-(4-methylpiperazin-1-ylmethyl)phenyl, 5′-carbamimidoyl-2′-hydroxyphenyl, 5′-(2-dimethylaminoethyl-aminocarbonyl)-2′-hydroxyphenyl, 3′-aminosulfonyl, 2′-hydroxy-5′-aminosulfonylphenyl, 2′-hydroxy-5′-hydroxymethylphenyl, 2′-hydroxy-5′-(2-hydroxyethyl)phenyl, 2′-hydroxy-5′-dimethylaminosulfonylaminophenyl, or 5′-aminocarbonyl-2′-hydroxyphenyl. More preferably, 2′,6′-dihydroxyphenyl, 5′-fluoro-2′-hydroxy-phenyl, 5′-aminocarbonyl-2′-hydroxyphenyl, 3′-aminosulfonylphenyl, 2′-hydroxy-5′-hydroxymethylphenyl, or 2′-hydroxy-5′-ureidomethylphenyl.

IV.

Yet another preferred group of compounds of Formula I are those wherein the moiety:

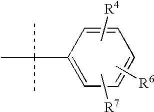

is 3′-acetylphenyl, 3′-hydroxyphenyl, 2′-hydroxyphenyl, 3′-aminocarbonylphenyl, 3′-cyanophenyl, 5′-fluoro-2′-hydroxyphenyl, 5′-chloro-2′-hydroxyphenyl, 2′-hydroxy-methylphenyl, 2′-hydroxyphenyl, 5′-carboxy-2′-hydroxyphenyl, 2′,5′-dihydroxyphenyl, 5′-cyano-2′-methoxyphenyl, 5′-aminocarbonyl-2′-methoxyphenyl, 2′,6′-dihydroxyphenyl, 2′-hydroxy-5′-nitrophenyl, 2′-cyanophenyl, 3′-hydroxymethylphenyl, 5′-cyano-2′-hydroxy-phenyl, 5′-aminocarbonyl-2′-hydroxyphenyl, 2′,6′-dihydroxyphenyl, 5′-aminomethyl-2′-hydroxyphenyl, 2′-hydroxy-5′-ureidomethylphenyl, 2′-hydroxy-5′-imidazol-2-ylphenyl, 5′-amino-2′-hydroxyphenyl, 2′-hydroxy-5′-ureidophenyl, 2′-hydroxy-5′-(2-morpholin-4-ylethyl)aminocarbonyl-phenyl, 3′-bromo-2′-hydroxy-5′-hydroxymethylphenyl, 5′-(2-cyanoethyl)-2′-hydroxyphenyl, 3′-bromo-5′-carboxymethyl-2′-hydroxyphenyl, 5′-(2-carboxyethyl)-2′-hydroxyphenyl, 5′-aminocarbonylmethyl-2′-hydroxyphenyl, 3′,5′-dichloro-2′-hydroxyphenyl, 2′-hydroxy-5′-[2-(2-hydroxyethoxy)ethylaminocarbonyl]phenyl, 5′-dimethylaminosulfonylamino-2′-hydroxy-phenyl, 3′-bromo-5′-chloro-2′-hydroxyphenyl, 2′-hydroxy-5′-(4-methylpiperazin-1-ylcarbonyl)phenyl, 2′-hydroxy-5′-(4-methylpiperazin-1-ylemthyl)phenyl, 5′-amidino-2′-hydroxyphenyl, 5′-(2-dimethylaminoethylaminocarbonyl)-2′-hydroxyphenyl, or 5′-aminocarbonyl-2′-hydroxyphenyl. Preferably 2′-hydroxyphenyl, 5′-fluoro-2′-hydroxyphenyl, 5′-chloro-2′-hydroxyphenyl, 2′-hydroxymethylphenyl, 2′-hydroxyphenyl, 5′-carboxy-2′-hydroxyphenyl, 2′,5′-dihydroxyphenyl, 2′,6′-dihydroxy-phenyl, 2′-hydroxy-5′-nitrophenyl, 5′-cyano-2′-hydroxyphenyl, 5′-aminocarbonyl-2′-hydroxyphenyl, 2′,6′-dihydroxyphenyl, 5′-aminomethyl-2′-hydroxyphenyl, 2′-hydroxy-5′-ureidomethylphenyl, 2′-hydroxy-5′-imidazol-2-ylphenyl, 5′-amino-2′-hydroxyphenyl, 2′-hydroxy-5′-ureidophenyl, 2′-hydroxy-5′-(2-morpholin-4-ylethyl)aminocarbonyl-phenyl, 3′-bromo-2′-hydroxy-5′-hydroxymethylphenyl, 5′-(2-cyanoethyl)-2′-hydroxyphenyl, 3′-bromo-5′-carboxymethyl-2′-hydroxyphenyl, 5′-(2-carboxyethyl)-2′-hydroxyphenyl, 5′-aminocarbonylmethyl-2′-hydroxyphenyl, 3′,5′-dichloro-2′-hydroxyphenyl, 2′-hydroxy-5′-[2-(2-hydroxyethoxy)ethylaminocarbonyl]phenyl, 5′-dimethylaminosulfonylamino-2′-hydroxy-phenyl, 3′-bromo-5′-chloro-2′-hydroxyphenyl, 2′-hydroxy-5′-(4-methylpiperazin-1-ylcarbonyl)phenyl, 2′-hydroxy-5′-(4-methylpiperazin-1-ylmethyl)phenyl, 5′-carbamimidoyl-2′-hydroxyphenyl, 5′-(2-dimethylaminoethylaminocarbonyl)-2′-hydroxyphenyl, or 5′-aminocarbonyl-2′-hydroxyphenyl. More preferably, 2′,6′-dihydroxyphenyl, 5′-fluoro-2′-hydroxy-phenyl, 5′-aminocarbonyl-2′-hydroxyphenyl, or 2′-hydroxy-5′-ureidomethylphenyl.

With the above preferred group of compounds III and IV, a more preferred group of compounds is that wherein:

X¹ is —N— and X², X³, and X⁴ are —CR⁵— where R⁵ is hydrogen, and R¹³ is hydrogen; or

X¹ is —N—; X² and X⁴ are —CR⁵— where R⁵ is hydrogen and X³ is —CR⁵— where R⁵ is halo, preferably fluoro or chloro, and R¹³ is hydrogen; or

X¹ is —CH— and X², X³, and X⁴ are —CR⁵— where R⁵ is hydrogen, and R¹³ is hydrogen; or

X¹ is —CH—; X² and X⁴ are —CR⁵— where R⁵ is hydrogen and X³ is —CR⁵— where R⁵ is halo, preferably fluoro or chloro, and R¹³ is hydrogen.

Within the above preferred and more preferred groups, an even more preferred group of compounds is that wherein R¹, R² and R¹³ are hydrogen; R³ is a group of formula (a) wherein n is 0; R⁸ and R¹⁰ are hydrogen; and R⁹ and R¹¹ are independently hydrogen or alkyl, preferably hydrogen, methyl, or ethyl. More preferably one of R⁹ and R¹¹ is hydrogen and the other of R⁹ and R¹¹ is ethyl.

Another more preferred group of compounds is that wherein R¹ and R² are hydrogen; R¹³ is hydroxy; and R³ is a group of formula (a) wherein n is 0; R⁸ and R¹⁰ are hydrogen; and R⁹ and R¹¹ are independently hydrogen or alkyl, preferably hydrogen, methyl, or ethyl. More preferably one of R⁹ and R¹¹ is hydrogen and the other of R⁹ and R¹¹ is ethyl.

Another more preferred group of compounds is that wherein R¹ and R² are hydrogen; R¹³ is hydrogen; R³ is a group of formula (a) wherein n is 0; R⁸ and R¹⁰ are hydrogen; and R⁹ and R¹¹ are aryl, aralkyl, or haloalkyl, preferably phenyl, benzyl or —CH₂CCl₃.

Another more preferred group of compounds is that wherein R¹ and R² are hydrogen; R¹³ is hydroxy; and R³ is a group of formula (a) wherein n is 0; R⁸ and R¹⁰ are hydrogen; and R⁹ and R¹¹ are aryl, aralkyl, or haloalkyl, preferably phenyl, benzyl or —CH₂CCl₃.

Another more preferred group of compounds is that wherein R¹, R² and R¹³ are hydrogen; R³ is a group of formula (a) wherein n is 0; R⁸, R⁹, R¹⁰ and R¹¹ are hydrogen.

Another more preferred group of compounds is that wherein R¹ and R² are hydrogen; R³ is a group of formula (a) wherein n is 0; R⁸, R⁹, R¹⁰ and R¹¹ are hydrogen; and R¹³ is hydroxy.

Yet another more preferred group of compounds of Formula I is that wherein R¹, R² and R¹³ hydrogen; R is a group of formula (a) wherein n is 0; R⁸ and R¹⁰ together from a covalent bond; and R⁹ and R¹¹ are independently hydrogen, methyl or ethyl, preferably hydrogen.

Yet another more preferred group of compounds is that wherein R¹, R² and R¹³ are hydrogen; R³ is a -(alkylene)-COOR⁹ where R⁹ is hydrogen or alkyl. Preferably R³ is —CH₂COOR⁹, —(CH₂)₂COOR⁹ wherein R⁹ is hydrogen, methyl or ethyl.

Yet another more preferred group of compounds is that wherein R¹ and R² are hydrogen; R¹³ is hydroxy, and R³ is a -(alkylene)-COOR⁹ where R⁹ is hydrogen or alkyl. Preferably R³ is —CH₂COOR⁹, —(CH₂)₂COOR⁹ wherein R⁹ is hydrogen, methyl or ethyl, preferably hydrogen or ethyl.

Yet another more preferred group of compounds is that wherein R¹, R² and R¹³ are hydrogen; R³ is a -(alkylene)-COOR⁹ where R⁹ is aryl, aralkyl, or haloalkyl. Preferably R³ is —CH₂COOR⁹, —(CH₂)₂COOR⁹ wherein R⁹ is phenyl, benzyl or —CH₂CCl₃.

Yet another more preferred group of compounds is that wherein R¹ and R² are hydrogen; R¹³ is hydroxy, and R³ is a -(alkylene)-COOR⁹ where R⁹ is aryl, aralkyl, or haloalkyl. Preferably R³ is —CH₂COOR⁹, —(CH₂)₂COOR⁹ wherein R⁹ is phenyl, benzyl or —CH₂CCl₃.

Reference to the preferred embodiments set forth above is meant to include all combinations of particular and preferred groups unless stated otherwise.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula I in which X¹ is —N—, R³ is a group of formula (a) where n is 0, R¹³ is hydrogen and X², X³, X⁴, R¹, R², R⁴-R¹¹ are as defined in the Summary of the Invention can be prepared as described in Scheme I below.

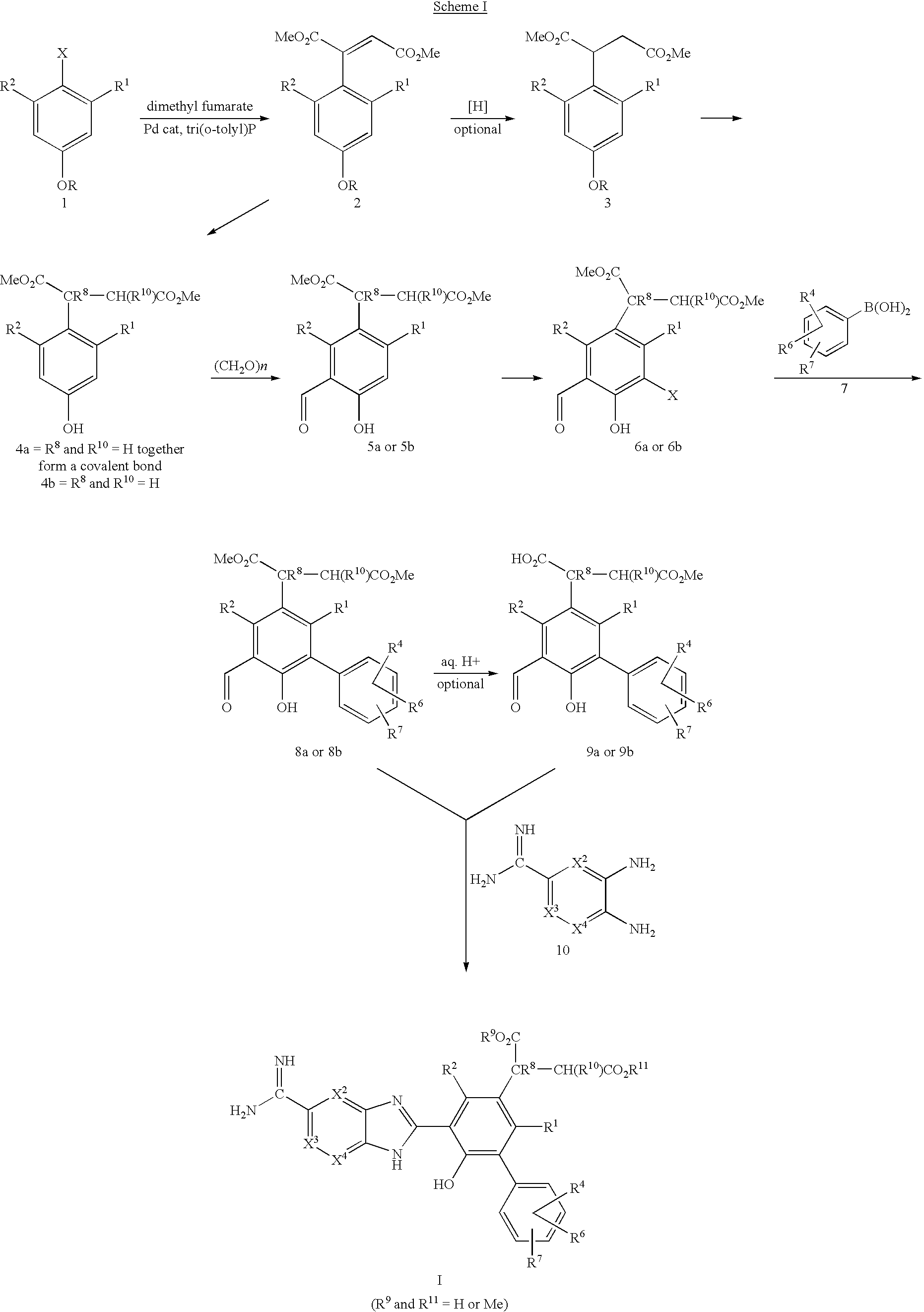

Reaction of a phenol derivative of formula 1 where R is hydrogen, alkyl or other suitable oxygen protecting group, X is halo, and R¹ and R² are as defined in the Summary of the Invention with fumarate diester such as dimethyl fumarate in the presence of a palladium (II) catalyst such as palladium acetate and tri(o-tolyl)phosphine or triphenylphosphine provides a (E)-2-phenyl-but-2-enedioic acid dimethyl ester compound of formula 2. The reaction is carried out in a suitable organic solvent such as acetonitrile, toluene, dimethylformamide, and the like, and in the presence of an organic base such as triethylamine, and the like.

Compounds of formula 1 are commercially available or they can be prepared by methods well known in the art. For example, 4-iodoanisole and 4-iodophenol are commercially available.

Compound 2 can be optionally reduced under hydrogenation reaction conditions to provide a 2-phenyl-succinic acid dimethyl ester compound of formula 3.

Compound 2 or 3 (where R is other than hydrogen) is then converted to the corresponding (E)-2-(4-hydroxyphenyl)-but-2-enedioic acid dimethyl ester (R⁸ and R¹⁰ form covalent bond) or 2-(4-hydroxyphenyl)-succinic acid dimethyl ester (R⁸ and R¹⁰ are hydrogen) compound of formula 4a or 4b respectively, by removal of the R group. The reaction conditions employed for the removal if R group depends on the nature of the R group. For example, if R is alkyl, it is removed by dealkylating agents such as hydrobromic acid, boron tribromide, and the like.

Treatment of 4a or 4b with paraformaldehyde under standard reaction conditions provides a (E)-2-(3-formyl-4-hydroxyphenyl)-but-2-enedioic dimethyl ester or 2-(3-formyl-4-hydroxyphenyl)-succinic acid dimethyl ester compound of formula 5a or 5b, respectively. Compound 5a or 5b is then converted to a compound of formula 6a or 6b where X is halo, preferably bromo or iodo with a suitable halogenating agent such as N-bromosuccinimide, N-iodosuccinimide, and the like. The reaction is carried out in a suitable organic solvent such as dimethylformamide.

A compound of formula 6a or 6b is then treated with a phenyl boronic acid of formula 7 to provide a (E)-2-(5-formyl-6-hydroxybiphenyl-3-yl)-but-2-enedioic or 2-(5-formyl-6-hydroxybiphenyl-3-yl)-succinic acid dimethyl ester compound of formula 8a or 8b respectively, which can be optionally converted to the corresponding diacid compound of formula 9a or 9b under aqueous acidic or basic hydrolysis reaction conditions.

Alternatively, a compound of formula 6a or 6b can be converted to a boronic acid derivative by methods well known in the art and the resulting boronic acid can then be coupled with a halobenzene of the formula Ph(R⁴, R⁶, R⁷)X where X is halo and R⁴-R⁷ are as defined in the Summary of the Invention under the conditions described above to provide a compound of formula 8a or 8b respectively.

A compound or formula 8(a or b) or 9(a or b) is then condensed with a 1,2-diamino compound of formula 10 to provide a compound of Formula I where X¹ is —N—. The reaction is carried out in the presence of a suitable oxidant such as benzoquinone, air oxidation, or FeCl₃ and O₂ and in a suitable organic solvent such as methanol, ethanol, and the like.

Compounds of formula 10 are commercially available or they can be prepared by methods well known in the art. For example, synthesis of 3,4-diaminobenzamidine monohydrochloride is known in the art.

Compounds of Formula I can be converted to other compounds of Formula I. For example, a compound of Formula I where R⁴ is alkoxy, can be converted to corresponding compound of Formula I where R⁴ is hydroxy by hydrolysis of the alkoxy group by a suitable dealkylating reagent such as hydrobromic acid, and the like. A compound of Formula I where R⁷ is cyano can be converted to a corresponding compound of Formula I where R⁷ is aminocarbonyl under hydrolysis reaction conditions. The cyano group can also be reduced to give aminomethyl group which can be treated with isocyanate or thiocyanate to give corresponding compound of Formula I where R⁷ is ureidomethyl or thioureidomethyl respectively. A compound of Formula I where R¹³ is hydrogen can be converted to a corresponding compound of Formula I where R¹³ is hydroxy or alkoxy by reacting it with hydroxylamine or alkoxyamine under conditions well known in the art.

Compounds of Formula I in which X¹ is —CH—, R³ is a group of formula (a) where n is 0, R¹³ is hydrogen, and X², X³, X⁴, R¹, R², R⁴-R¹¹ are as defined in the Summary of the Invention can be prepared as described in Scheme II below.

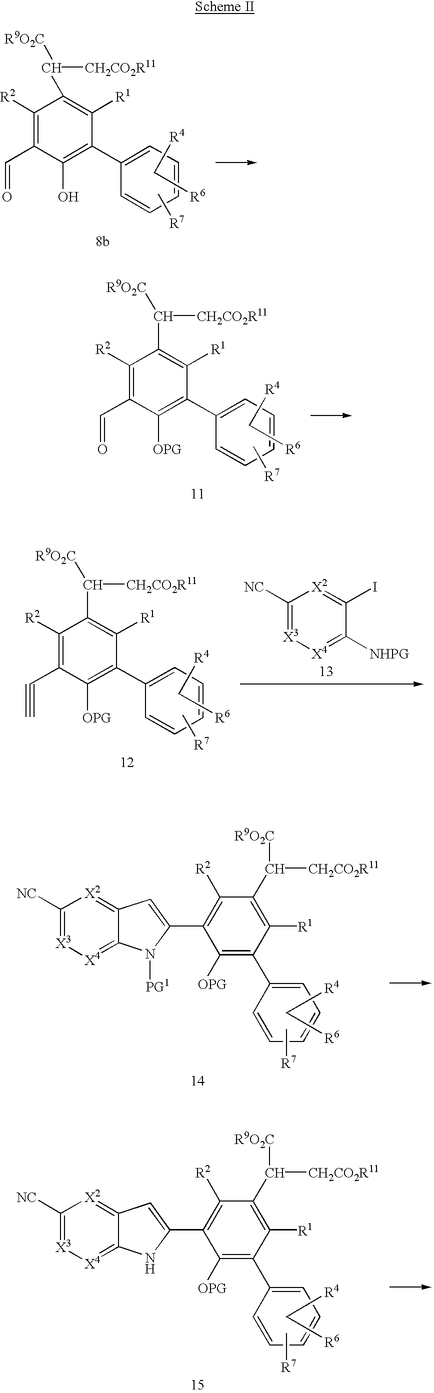

Protection of the hydroxy group in a compound of formula 8a where R⁹ and R¹¹ are alkyl, prepared as described in Scheme I above, with a suitable hydroxy protecting group provides a compound of formula 11. A comprehensive list of suitable hydroxy protective groups can be found in T. W. Greene, Protective Groups in Organic Synthesis, John Wiley & Sons, Inc. 1981, the disclosure of which is incorporated herein by reference in its entirety. Preferred hydroxy protecting group is 2-methoxyethoxymethyl. The reaction is typically carried out in the presence of a base such as diispropylethylamine, and the like and in a halogenated organic solvent such as dichloromethane, carbon tetrachloride, chloroform, and the like.

Ethynylation of 11 utilizing a modified procedure described in Muller, S.; Liepold, B.; Roth G. J.; Bestmann H. J. Synlett 1996, 6, 521-522 provides a 2-(5-ethynylbiphenyl-3-yl)-succinic acid dialkyl ester compound of formula 12. A detailed description of this procedure is provided in working examples below.

Reaction of a compound of formula 11 with a cyano compound of formula 13 where PG¹ is a suitable nitrogen protecting group such as methylsulfonyl, tert-butoxycarbonyl, trifluoroacetyl, and the like, utilizing the reaction conditions described in Sakamoto, T; Kondo, Y.; Iwashita, S.; Nagano, T.; Yamanaka, H. Chem. Pharm. Bull. 1988, 36, 1305 provides 2-[5-(5-cyanoindol-2-yl)biphenyl-3-yl]-succinic acid dialkyl ester compound of formula 14 (where X¹, X², X³ and X⁴ are carbon). Deprotection of the amino group in 14 provides a 2-[5-(5-cyano-1H-indol-2-yl)biphenyl-3-yl]-succinic acid dialkyl ester compound of formula 15. The reaction conditions utilized in the deprotection step depends on the nature of the nitrogen protecting group. For example, if the protecting group is methylsulfonyl it is removed under basic hydrolysis reaction conditions. Suitable bases are aqueous sodium hydroxide, potassium hydroxide, and the like. The reaction is carried out in an alcoholic solution such as methanol, ethanol, and the like. If the protecting group is tert-butoxycarbonyl it is removed under acidic hydrolysis reaction conditions. Compounds of formula 13 are either commercially available or they can be prepared by methods well known in the art.

The hydroxy-protecting group in 15 is then removed to provide 2-[5-(5-cyanoindol-2-yl)-6-hydroxybiphenyl-3-yl]-succinic acid dialkyl ester 16. The reaction conditions employed for the deprotection reaction depend on the nature of the hydroxy protecting group. For example, if the protecting group is 2-methoxyethoxymethoxy, it is removed by treating 15 with an acid under non-aqueous reaction conditions, in a suitable alcoholic solvent.

The cyano group in compound 16 is then converted into the amidino group by first treating 16 with hydrogen chloride gas in an anhydrous alcoholic solvent such as methanol, ethanol and the like, and then treating the resulting 2-[5-(5-methoxycarbonimidolyl-1H-indol-2-yl)-6-hydroxybiphenyl-3-yl]-succinic acid dialkyl ester 17 with an inorganic base such as ammonium carbonate, and the like in an alcoholic solvent such as methanol, ethanol, or with excess ammonia to give resulting 2-[5-(5-carbamimidolyl-1H-indol-2-yl)-6-hydroxybiphenyl-3-yl]-succinic acid dialkyl ester of Formula I. 2-[5-(5-Carbamimidolyl-1H-indol-2-yl)-6-hydroxybiphenyl-3-yl]-succinic acid dialkyl ester of Formula I can be converted to a corresponding compound of Formula I where R⁹ and R¹¹ are hydrogen under hydrolysis conditions well known in the art.

The above procedure can also be used to prepare compounds of Formula I where R⁸ and R¹⁰ together form a covalent bond. The compounds of Formula I can also be prepared by synthetic procedures described in Applicant's PCT Application Publication No. WO 00/35886 the disclosure of which is incorporated herein by reference in its entirety.

### Utility

The compounds of this invention inhibit Factors VIIa, IXa, Xa, and XIa, in particular Factor VIIa, and are therefore useful as anticoagulants for the treatment or prevention of thromboembolic disorders in mammals.

Particular disease states which may be mentioned include the therapeutic and/or prophylactic treatment of venous thrombosis (e.g. DVT) and pulmonary embolism, arterial thrombosis (e.g. in myocardial infarction, unstable angina, thrombosis-based stroke and peripheral arterial thrombosis), and systemic embolism usually from the atrium during atrial fibrillation or from the left ventricle after transmural myocardial infarction, or caused by congestive heart failure; prophylaxis of reocclusion (i.e., thrombosis) after thrombolysis, percutaneous trans-luminal angioplasty (PTA) and coronary bypass operations; the prevention of rethrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosclerosis such as coronary arterial disease and the formation of atherosclerotic plaques, cerebral arterial disease, cerebral infarction, cerebral thrombosis, cerebral embolism, peripheral arterial disease, ischaemia, angina (including unstable angina), reperfusion damage, restenosis after percutaneous trans-luminal angioplasty (PTA) and coronary artery bypass surgery.

The compounds of Formula I can also be used in the treatment of cancer.

### Testing

The ability of the compounds of this invention to inhibit factor VIIa and Xa can be tested in vitro and in vivo assays described in biological assays Example 1 and 2 below.

### Administration and Pharmaceutical Compositions

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of compounds of Formula I may range from approximately 0.01-50 mg per kilogram body weight of the recipient per day; preferably about 0.1-20 mg/kg/day. Thus, for administration to a 70 kg person, the dosage range would most preferably be about 7 mg to 1.4 g per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral or parenteral using a convenient daily dosage regimen, which can be adjusted according to the degree of affliction. Oral compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula I in combination with at least one pharmaceutically acceptable excipient. Acceptable excipients are non-toxic, aid administration, and do not adversely affect the therapeutic benefit of the compound of Formula I. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula I based on the total formulation, with the balance being one or more suitable pharmaceutical excipients. Preferably, the compound is present at a level of about 1-80 wt %. Representative pharmaceutical formulations containing a compound of Formula I are described below.

The compounds of Formula I can be administered alone or in combination with other compounds of Formula I or in combination with one or more other active ingredient(s). For example, a compound of Formula I can be administered in combination with another anticoagulant agent(s) independently selected from a group consisting of a thrombin inhibitor, a factor IXa, and a factor Xa inhibitor. Preferably, the thrombin inhibitor is Inogatran®, Melagatran® or prodrugs thereof which are disclosed in PCT Application Publication Nos. WO 94/29336 and WO 97/23499, the disclosures of which are incorporated herein by reference in their entirety. Factor Xa inhibitors that may be used in the combination products according to the invention include those described in Current Opinion in Therapeutic Patents, 1993, 1173-1179 and in international patent applications WO 00/20416, WO 00/12479, WO 00/09480, WO 00/08005, WO 99/64392, WO 99/62904, WO 99/57096, WO 99/52895, WO 99/50263, WO 99/50257, WO 99/50255, WO 99/50254, WO 99/48870, WO 99/47503, WO 99/42462, WO 99/42439, WO 99/40075, WO 99/37304, WO 99/36428, WO 99/33805, WO 99/33800, WO 99/32477, WO 99/32454, WO 99/31092, WID 99/26941, WO 99/26933, WO 99/26932, WO 99/26919, WO 99/26918, WO 99/25720, WO 99/16751, WO 99/16747, WO 99/12935, WO 99/12903, WO 99/11658, WO 99/11617, WO 99/10316, WO 99/07732, WO 9/07731, WO 99/05124, WO 99/00356, WO 99/00128, WO 99/00127, WO 99/00126, WO 9/00121, WO 98/57951, WO 98/57937, WO 98/57934, WO 98/54164, WO 98/46591, WO 98/31661, WO 98/28282, WO 98/28269, WO 98/25611, WO 98/24784, WO 98/22483, WO 98/16547, WO 98/16525, WO 98/16524, WO 98/16523, WO 98/15547, WO 98/11094, WO 98/07725, WO 98/06694, WO 98/01428, WO 7/48706, WO 97/46576, WO 97/46523, WO 97/38984, WO 97/30971, WO 97/30073, WO 97/29067, WO 97/24118, WO 97/23212, WO 97/21437, WO 97/08165, WO 97/05161, WO 96/40744, WO 96/40743, WO 96/40679, WO 96/40100, WO 96/38421, WO 96/28427, WO 96/19493, WO 96/16940, WO 95/28420, WO 94/13693, WO 00/24718, WO 99/55355, WO 99/51571, WO 99/40072, WO 99/26926, WO 98/51684, WO 97/48706, WO 97/24135, WO 97/11693, WO 00/01704, WO 00/71493, WO 00/71507, WO 00/71508, WO 00/71509, WO 00/71511, WO 00/71512, WO 00/71515, WO 00/71516, WO 00/13707, WO 00/31068, WO 00/32590, WO 00/33844, WO 00/35859, WO 00/35886, WO 00/38683, WO 00/39087, WO 00/39092, WO 00/39102, WO 00/39108, WO 00/39111, WO 00/39117, WO 00/39118, WO 00/39131, WO 00/40548, WO 00/40571, WO 00/40583, WO 00/40601, WO 00/47207, WO 00/47553, WO 00/47554, WO 00/47563, WO 00/47578, WO 00/51989, WO 00/53264, WO 00/59876, WO 00/59902, WO 00/71510, WO 00/76970, WO 00/76971, WO 00/78747, WO 01/02356, WO 01/02397, WO 01/05784, WO 01/09093, WO 01/12600, WO 01/19788, WO 01/19795, WO 01/19798, WO 93/15756, WO 94/17817, WO 95/29189, WO 96/18644, WO 96/20689, WO 96/39380, WO 97/22712, WO 97/36580, WO 97/36865, WO 97/48687, WO 98/09987, WO 98/46626, WO 98/46627, WO 98/46628, WO 98/54132, WO 99/07730, WO 99/33458, WO 99/37643 and WO 99/64446; in U.S. Pat. Nos. 6,034,093, 6,020,357, 5,994,375, 5,886,191, 5,849,519, 5,783,421, 5,731,315, 5,721,214, 5,693,641, 5,633,381, 5,612,378, 6,034,127, 5,670,479, 5,658,939, 5,658,930, 5,656,645, 5,656,600, 5,639,739, 5,741,819, 6,057,342, 6,060,491, 6,080,767, 6,087,487, 6,140,351, 6,395,731, and 5,646,165; in Japanese patent applications Nos. JP 99152269, JP 10017549, JP 10001467, JP 98017549, JP 00178243, JP 11140040, JP 12143623, JP 12204081, JP 12302765, JP 6327488 and JP 98001467; in European patent applications EP 937 723, EP 937 711, EP 874 629, EP 842 941, EP 728 758, EP 540 051, EP 419 099, EP 686 642, EP 1 016 663 and EP 529 715; and in German patent applications Nos. DE 19845153, DE 19835950, DE 19743435, DE 19829964, DE 19834751, DE 19839499, DE19900355, DE19900471 and DE 19530996, the specific and generic disclosures in all of which documents are hereby incorporated by reference.

Factor Xa inhibitors also include those disclosed in international patent applications WO 96/10022, WO 97/28129, WO 97/29104, WO 98/21188, WO 99/06371, WO 99/57099, WO 99/57112, WO 00/47573, WO 00/78749, WO 99/09027 and WO 99/57113, the specific and generic disclosures in all of which documents are hereby incorporated by reference, as well as 4-{4-[4-(5-chloroindol-2-ylsulfonyl) piperazine-1-carbonyl]phenyl}-pyridine-1-oxide and pharmaceutically acceptable derivatives thereof. Preferred Factor Xa inhibitors include antistatin, tick anticoagulant protein and those known as SQ-311 and SQ-315 (see international patent application WO 98/57951); SN-292 (see international patent application WO 98/28282); SN-429 and SN 116 (see international patent application WO 98/28269); RPR-208707 (see international patent application WO 98/25611 at Example 48); XU-817 (see international patent application WO 98/01428); SF-324 and SF-303 (see international patent application WO 97/23212); YM 60828 (see international patent application WO 96/16940 at Example 75); FACTOREX (see U.S. Pat. No. 5,783,421); SF-324 (see European patent application EP 874 629); DX9065A (see European patent application EP 540 051 at Example 39); 1-(4-amidinobenzyl)-4-(6-chloronaphthalene-2-ylsulfonyl)-piperazin-2-one (see JP 12204081 at Example 2); M55555 (see international patent application WO 99/33805 at Example 39); DPC423 (1-(3-amidinophenyl)-2-(2′-aminolsulfonyl[1,1′-biphenyl]-4-ylaminocarbonyl)-4-bromopyrrole, see international patent application WO 98/28269); 3-(3,5-difluoro-6-[3-(4,5dihydro-1-methylimidazol-2-yl)-phenoxy]-4-[2,3-dihydroxy-propoxy]-pyridin-2-yloxy)-4-hydroxybenzamidine (see international patent application WO 00/31068); ZK-807834 (see international patent application WO 7/29067); 1,4-diaza-4-(6-chloro-naphthalene-2ylsulfonyl)-6-(methoxymethyl)-7-oxa-1′-(pyridin-4-yl)spiro[bicyclo-[4-3.0]-nonane-8,4′-piperidine]-2-one (see international patent application WO 01/02397); (S)-1-(4-aminoquinazolin-7-ylmethyl)-4-[2-(5-chlorothien-2-yloxy)acetyl]-3-methoxy-methylpiperazin-2-one (see international patent application WO 00/32590); 3-(2-[4-(2-aminosulfonyl-phenyl)benzoylphenoxy)-benzamidine (see international patent application WO 01/19788); and 4-(2-[4-(5-chloroindol-2-yl-sulfonyl)-2-(pyrrolidin-1-ylcarbonylmethyl)piperazin-1-yl-carbonyl]-thiazol-5-yl)pyridine N-oxide (see Japanese patent application No. JP 12143623); as well as the compounds of Example 7 of international patent application WO 98/21188, of Examples 3 and 6 of WO 99/57113, of Example 6 of international patent application WO 00/78747, of Examples 188, 211 and 167 of U.S. Pat. No. 6,080,767, of Examples 40, 54 and 55 of international patent application WO 99/33805, of Examples 5, 6, 8, 9, 10, 11, 12, 13, 15, 16 and 17 of international patent application WO 01/05784, of Examples 6, 7, 8, 9, 10, 11, 12, 13, 14, 15, 16, 17, 22, 23, 25, 26, 28, 29, 30, 31, 32, 33, 34, 38, 39, 40, 41, 42 and 43 of international patent application WO 01/12600, and of Examples 802 and 877 of international patent application WO 00/35886. Other anticoagulant agents that can be used in the combination therapy are those disclosed in U.S. Patent Applications Publication Nos. 20020065303, 20020061842, 20020058677, 20020058657, 20020055522, 20020055469, 20020052368, 20020040144, 20020035109, 20020032223, 20020028820, 20020025963, 20020019395, 20020019394, 20020016326, 20020013314, 20020002183, 20010046974, 20010044537, 20010044536, 20010025108, 20010023292, 20010023291, 20010021775, 20010020020033, 20010018423, 20010018414, and 20010000179, which are incorporated herein by reference in their entirety.

Suitable formulations for use in administering melagatran and derivatives (including prodrugs) thereof are described in the literature, for example as described in inter alia international patent applications WO 94/29336, WO 96/14084, WO 96/16671, WO 97/23499, WO 97/39770, WO 97/45138, WO 98/16252, WO 99/27912, WO 99/27913, WO 00/12043 and WO 00/13671, the disclosures in which documents are hereby incorporated by reference.

Similarly, suitable formulations for use in administering Factor Xa inhibitors and derivatives (including prodrugs) thereof are described in the literature, for example as described in the prior art documents relating to Factor Xa inhibitors that are mentioned hereinbefore, the disclosures in which documents are hereby incorporated by reference. Otherwise, the preparation of suitable formulations, and in particular combined preparations including both melagatran/derivative and Factor Xa inhibitor/derivative may be achieved non-inventively by the skilled person using routine techniques. The amounts of melagatran, Factor Xa inhibitor, or derivative of either, in the respective formulation(s) will depend on the severity of the condition, and on the patient, to be treated, as well as the compound(s) which is/are employed, but may be determined non-inventively by the skilled person.

Suitable doses of melagatran, Factor Xa inhibitors and derivatives of either, in the therapeutic and/or prophylactic treatment of mammalian, especially human, patients may be determined routinely by the medical practitioner or other skilled person, and include the respective doses discussed in the prior art documents relating to melagatran (or derivatives (including prodrugs) thereof), and to Factor Xa inhibitors, that are mentioned hereinbefore, the disclosures in which documents are hereby incorporated by reference.

### EXAMPLES

The following preparations and examples are given to enable those skilled in the art to more clearly understand and to practice the present invention. They should not be considered as limiting the scope of the invention, but merely as being illustrative and representative thereof.

### Synthetic Examples

### Reference 1

### Synthesis of 5-fluoro-2-hydroxyphenyl-boronic acid

A 250 mL 24/40 round bottom flask was charge with 5-fluoro-2-methoxyphenyl-boronic acid (2.2 g, 12.94 mmol) and a magnetic stir bar. A 1M solution of tribromoborane in dichloromethane (26 mL) was added to the flask and the mixture was stirred for 18 hours. The reaction was quenched by the slow addition of water (50 mL) and the resulting mixture was extracted with ethyl acetate. The organic layer was concentrated via rotoevaporation to give 5-fluoro-2-hydroxyphenyl-boronic acid (2.0 g) as a white solid.

### Reference 2

### Synthesis of 2-(3-bromo-5-formyl-4-hydroxy-phenyl)-succinic acid dimethyl ester

Step (a)

A solution of 1-iodo-4-methoxy-benzene (48.4 g, 0.207 mol) in acetonitrile (55 mL) was mixed with triethylamine (29.0 mL, 0.207 mol), Pd(OAc)₂ (0.264 g, 2.07 mmol ) and tri(o-tolyl)phosphine (1.26 g, 4.14 mmol) followed by (E)-but-2-enedioic acid dimethyl ester (42.44 mL, 0.26 mol). The resulting mixture was refluxed for three hours and then was combined with water/ether. The mixture was extracted with ether (×2) and the extract dried (MgSO₄) and then concentrated under reduced pressure. The residue was purified by column chromatography (600 g silica/EtOAc/hexane) to yield (E)-2-(4-methoxy-phenyl)-but-2-enedioic acid dimethyl ester (87% yield).

Step (b)

A solution of (E)-2-(4-methoxy-phenyl)-but-2-enedioic acid dimethyl ester (12.5 g, 45 mmol) in ethanol (250 mL) was mixed with Pearlman's catalyst (400 mg) and the resulting mixture was hydrogenated for approximately 15 hours. The reaction mixture then was filtered through silica and the ethanol filtrate was concentrated under reduced pressure to yield 2-(4-methoxy-phenyl)-succinic acid dimethyl ester (99% yield).

Step (c)

A mixture of 2-(4-methoxy-phenyl)-succinic acid dimethyl ester (30.0 g, 0.107 mol) and 48% aqueous HBr (250 mL) was heated for 4 hours at 120° C. The mixture was concentrated under reduced pressure. The residue was mixed with methanol (500 mL) and then thionyl chloride (10 mL). The mixture was heated approximately 4 hours at a temperature of approximately 60° C. The mixture was concentrated and the residue was mixed with aqueous sodium bicarbonate. The aqueous mixture was extracted with methylene chloride (×3) and the combined extracts were dried (MgSO₄) and concentrated under reduced pressure to yield 2-(4-hydroxy-phenyl)-succinic acid dimethyl ester (92% yield).

Step (d)

A mixture of 2-(4-hydroxy-phenyl)-succinic acid dimethyl ester (11.90 g, 50.0 mmol) and dry acetonitrile (250 mL) was treated with anhydrous magnesium chloride (7.14 g, 75.0 mmol), TEA (26.13 mL, 0.1875 mol) and paraformaldehyde (10.51 g, 0.35 mol). The reaction mixture was refluxed for approximately 1 hour, cooled to ambient temperature and mixed with 1N HCl/ether. The organic layer was isolated and the aqueous layer was extracted with ether (×2). The combined organic extracts were dried (MgSO₄) and concentrated under reduced pressure. A mixture of the residue and dry acetonitrile (250 mL) was treated with anhydrous magnesium chloride (7.14 g, 75.0 mmol), TEA (26.13 mL, 0.1875 mol) and paraformaldehyde (10.51 g, 0.35 mol). The reaction mixture was refluxed for approximately 1 hour, cooled to ambient temperature and mixed with 1N HCl/ether. The organic layer was isolated and the aqueous layer was extracted with ether (×2). The combined organic extracts were dried (MgSO₄) and concentrated under reduced pressure to yield 2-(3-formyl-4-hydroxy-phenyl)-succinic acid dimethyl ester (89% yield).

Step (e)

A mixture 2-(3-formyl-4-hydroxy-phenyl)-succinic acid dimethyl ester (15.3 g, 57.45 mmol) and dry DMF (150 mL) was diluted, in a dropwise manner, with a solution of N-bromosuccinimide (11.3 g, 63.5 mmol) in DMF (75 mL). The mixture was agitated for about 2 hours and then concentrated under reduced pressure at less than 35° C. The residue was dissolved in ether and the mixture was washed with water (×3). The ether layer was dried (MgSO₄) and then concentrated to yield 2-(3-bromo-5-formyl-4-hydroxy-phenyl)-succinic acid dimethyl ester (99% yield).

¹H NMR (CDCl₃) δ ppm: 2.57 (d of d, J=6 Hz, 18 Hz, 1H), 3.04 (d of d, J=11 Hz, 18 Hz, 1H), 3.54 (s, 3H), 3.57 (s, 3H), 3.93 (d of d, J=6 Hz, 11 Hz, 1H), 7.35 (d, J=2 Hz, 1H), 7.59 (d, J=2 Hz, 1H), 9.70 (s, 1H), 11.41 (s, 1H). MS: found (MH+) 345.0, calc 344.99.

### Reference 3

### Synthesis of (E)-2-(3-formyl-4-hydroxy-5-iodo-phenyl)-but-2-enedioic acid dimethyl ester

Step (a)

A 1L 24/40 round bottom flask was charged with 4-iodophenol (18.46 g, 83.9 mmol), dimethyl fumarate (13.30 g, 82.30 mmol), tri-O-tolylphosphine (510 mg, 1.68 mmol), triethylamine (200 mL) and a magnetic stir bar. The reaction flask was sparged with nitrogen, sealed with a rubber septum and kept under an atmosphere of nitrogen throughout the reaction. The mixture was heated at 90° C. until all solids had dissolved and then palladium acetate (189 mg, 0.84 mmol) was added to the solution. The mixture was stirred with heating for 18 hours and then concentrated under reduced pressure to give a solid. The solid was combined with ethyl acetate (1L) which to give a suspension. The suspension was washed with 1N aqueous hydrochloric acid, saturated aqueous NaHCO₃ and water, dried over MgSO₄, filtered and concentrated to give a gum (15.9 g). The residue was triturated with 1:1 ethyl acetate:hexanes to give (E)-2-(4-hydroxy-phenyl)-but-2-enedioic acid dimethyl ester (7.9 g, 33 mmol, 40% yield).

Step (b)

A 1L 24/40 round bottom flask was charged with (E)-2-(4-hydroxy-phenyl)-but-2-enedioic acid dimethyl ester (4.78 g, 20.24 mmol), acetonitrile (200 mL), MgCl₂ (2.89 g, 30.35 mmol), triethylamine (11 mL, 75.88 mmol) and a magnetic stir bar. The mixture was stirred and warmed to 50° C. and then paraformaldehyde (4.10 g, 136.59 mmol) was added to the mixture. The reaction mixture was heated to reflux, stirred for 2 hours, then cooled to ambient temperature and poured into 1L diethyl ether. The resulting mixture was washed with 1N hydrochloric acid, dried over MgSO₄, filtered and concentrated to an oil.

The oil was combined with acetonitrile (200 mL), MgCl₂ (2.89 g, 30.35 mmol) and triethylamine (11 mL, 75.88 mmol) in a 1L 24/40 round bottom flask with a magnetic stirring bar. The mixture was stirred and warmed to 50° C. and then paraformaldehyde (4.10 g, 136.59 mmol) was added to the mixture. The mixture was heated to reflux, stirred for 2 hours, then cooled to ambient temperature and poured into 1L diethyl ether. The resulting mixture was washed with 1N hydrochloric acid, dried over MgSO₄, filtered and concentrated to give (E)-2-(3-formyl-4-hydroxy-phenyl)-but-2-enedioic acid dimethyl ester (4.0 g, 15.14 mmol).

Step (c)

A 1 L round bottom flask was charged with (E)-2-(3-formyl-4-hydroxy-phenyl)-but-2-enedioic acid dimethyl ester (4.0 g, 15.14 mmol), DMF (100 mL) and a magnetic stir bar and fitted with an addition funnel. N-Iodosuccinimide (5.11 g, 22.71 mmol) in DMF was added dropwise via the addition funnel. The mixture was stirred for 2 hours and then was diluted with diethyl ether (500 mL). The resulting mixture was washed with water, dried over MgSO₄, filtered and concentrated to a dark solid. The residue was triturated with diethyl ether to give (E)-2-(3-formyl-4-hydroxy-5-iodo-phenyl)-but-2-enedioic acid dimethyl ester (3.0 g, 7.7 mmol, 51% yield) as a light yellow waxy solid. MS LCMS Q⁻ 388.960 (calc.), 389.1 (obs.), Q⁺ 390.968 (calc.).

### Reference 4

### Synthesis of 2-(5-formyl-6-hydroxy-3′-nitro-biphenyl-3-yl)-succinic acid

Step (a)

A mixture of 2-(3-bromo-5-formyl-4-hydroxy-phenyl)-succinic acid dimethyl ester (15.0 g, 43.5 mmol), prepared as in Reference 2 above, toluene (220 mL) and methanol (90 mL) was combined with 3-nitrophenylboronic acid (10.9 g, 65.3 mmol) and 2M aqueous sodium carbonate (33.0 mL, 66.0 mmol). The reaction flask was flushed with nitrogen and the reaction mixture then was mixed with tetrakis-(triphenylphosphine) palladium (5.1 g, 4.4 mmol) and the mixture was heated to reflux for about 7 hours. The mixture was cooled to ambient temperature and then mixed with 1M HCl. The organic layer was isolated, dried (MgSO₄) and evaporated. The residue was purified using column chromatography (300 g silica, EtOAc/hexane) to give 2-(5-formyl-6-hydroxy-3′-nitro-biphenyl-3-yl)-succinic acid dimethyl ester (66% yield).

Step (b)

A mixture of 2-(5-formyl-6-hydroxy-3′-nitro-biphenyl-3-yl)-succinic acid dimethyl ester (3.8 g, 10 mmol), 3 N aqueous HCl (60 mL) and acetonitrile (20 mL) was heated for approximately 4 hours. The reaction mixture was cooled to ambient temperature and then concentrated under reduced pressure. The residue was dried over phosphorus pentoxide under high vacuum to yield 2-(5-formyl-6-hydroxy-3′-nitro-biphenyl-3-yl)-succinic acid (98% yield). MS: found (M−H) 358.1, calc 359.06.

### Reference 5

### Synthesis of 2-[5-ethynyl-6-(2-methoxy-ethoxymethoxy)-3′-nitro-biphenyl-3-yl]-succinic acid dimethyl ester

Step (a)

A mixture of 2-(5-formyl-6-hydroxy-3′-nitro-biphenyl-3-yl)-succinic acid dimethyl ester (0.80 g, 2.06 mmol), prepared as in Reference 4, Step (a), dichloromethane (35 mL), and diisopropylethylamine (0.72 mL, 4.12 mmol) was cooled to approximately 5° C. The cooled mixture was diluted by a dropwise addition of MEM-chloride (0.35 mL, 3.09 mmol) and the resulting mixture was warmed to ambient temperature. The mixture then was agitated at ambient temperature from approximately 15 hours and then mixed with ethyl acetate and water. The organic layer was isolated, washed with water (×5), dried (MgSO₄) and concentrated under reduced pressure to afford 2-[5-formyl-6-(2-methoxyethoxymethoxy)-3′-nitro-biphenyl-3-yl]-succinic acid dimethyl ester.

Step (b)

A solution of 2-[5-formyl-6-(2-methoxyethoxymethoxy)-3′-nitrobipheny-3-yl]succinic acid dimethyl ester, methanol (12 mL), 1-diazo-2-oxopropyl)phosphinic acid dimethyl ester (0.63 g, 3.3 mmol) was treated with finely ground potassium carbonate (0.88 g, 3.3 mmol). This reaction mixture was stirred for approximately 30 min. The progress of the reaction was followed by monitoring the evolution of nitrogen and when complete the reaction was quenched by the addition of 5% citric acid. The mixture was extracted with ethyl acetate and the organic layer was passed through a pad of silica using 40% EtOAc/hexane as eluent. The organic layer was concentrated under reduced pressure to afford the compound of 2-[5-ethynyl-6-(2-methoxy-ethoxymethoxy)-3′-nitro-biphenyl-3-yl]-succinic acid dimethyl ester.

NMR (CDCl₃) δ ppm: 2.67 (d of d, J=6.0 Hz, 18.7 Hz, 1H), 3.10-3.30 (m, 8H), 3.67 (s, 3H), 3.69 (s, 3H ) , 4.05 (d of d, J=6.0 Hz, 10.7 Hz, 1H), 5.07 (s, 2H), 7.24-7.31 (m, 2H), 7.44 (d, J=2.4 Hz, 1H), 7.58 (t, J=9.0 Hz, 1H), 7.77-7.84 (m, 1H), 8.19 (d of d, J=2.4 9.0 Hz, 1H), 8.37 (t, J=1.9 Hz, 1H).

### Reference 6

### Synthesis of (E)-2-(5′-fluoro-5-formyl-6,2′-dihydroxy-biphenyl-3-yl)-but-2-enedioic acid dimethyl ester

A 250 mL 24/40 round bottom flask was charged with (E)-2-(3-formyl-4-hydroxy-5-iodo-phenyl)-but-2-enedioic acid dimethyl ester (0.544 g, 1.39 mmol), prepared as in Reference 3, 2-hydroxy-5-fluorophenyl-boronic acid (0.435 g, 2.79 mmol), prepared as in Reference 1, 1N K₂CO₃ (4.2 mL, 4.18 mmol), tetrakis-(triphenylphosphine) palladium (81 mg, 0.07 mmol), ethylene glycol dimethyl ether (50 mL) and a magnetic stir bar. The mixture was put under an atmosphere of nitrogen, stirred and heated to reflux for 4 hours. The mixture was neutralized with 1N hydrochloric acid and extracted with ethyl acetate. The organic layer was dried over MgSO₄, filtered and concentrated via rotoevaporation to a gum. Product was purified from the residue by chromatography on silica gel, elution with 40:60 ethyl acetate:hexane to give (E)-2-(5′-fluoro-5-formyl-6,2′-dihydroxy-biphenyl-3-yl)-but-2-enedioic acid dimethyl ester (100 mg, 0.27 mmol, 19% yield) as a yellow oil. MS LCMS Q⁻ 373.080 (calc.), 373.1 (obs.), Q⁺ 375.088 (calc).

### Reference 7

### Synthesis of 6-bromo-5-(tert-butoxycarbonylamino)-3-chloro-2-cyanopyridine

Step (a)

2-Hydroxy-5-nitropyridine (50 g, 357 mmol) and N-chlorosuccinimide (55 g, 410 mmol) were suspended in 150 mL anhydrous DMF. The reaction mixture was stirred at room temperature for 18 hours. The resulting homogeneous reaction mixture was dilluted by the slow addition of 750 mL of water, which resulted in the precipitation of the desired 3-chloro-5-nitro-2-hydroxypyridine as a pale yellow powder. The solids were isolated via filtration and further dried under high vacuum to provide 3-chloro-5-nitro-2-hydroxypyridine (59 g, 95% yield).

Step (b)

3-Chloro-5-nitro-2-hydroxy-pyridine (20 g) was added in small portions to thionylchloride (200 mL) under vigorous stirring. The suspension was heated to 100° C. within 1 h and stirred at 100° C. for 1 h. After cooling the solution to RT, the solvent was removed under reduced pressure, the residue dissolved in AcOEt, and washed with water (3× 200 mL). The organic layer was dried over MgSO₄, and the solvent was removed under reduced pressure. 2,3-Dichloro-5-nitropyridine (18 g) was obtained as a pale yellow solid.

Step (c)

A solution of 2,3-dichloro-5-nitropyridine (9.75 g) and KI (29 g) in HOAc (120 mL, degassed with N₂) was heated to 100° C. for 1.5 h under N₂. The brown solution was cooled to room temperature, AcOEt (300 mL) added and the organic phase washed with water (2 times 100 mL) and dilute aequ. Na₂SO₃ (100 mL). Evaporation of the solvent gave crystalline 3-chloro-2-iodo-5-nitro-pyridine (13.11 g).

Step (d)

A suspension of CuCN (7 g, Caution! Toxic HCN may be formed!) and 3-chloro-2-iodo-5-nitro-pyridine (7 g, Caution! Compound may detonated at elevated temperatures) in acetonitrile (200 mL) was heated to 80° C. within 1 h and stirred at 80° C. for 5 h. Evaporation of the solvent and filtration of the residue in AcOEt over SiO₂ gave 3-chloro-2-cyano-5-nitro-pyridine (4.26 g).

Step (e)

A solution of SnCl₂ (52 g) and 3-chloro-2-cyano-5-nitro-pyridine (10.3 g) was stirred in AcOEt (200 mL) at room temperature for 10 min and at 70° C. for 4 h. The solution was cooled to room temperature, diluted with AcOEt (500 mL), NaHCO₃ (100 g) added in four portions within 4 h, and vigorously stirred for 20 h. The suspension was filtered, the filtrate washed with sat. aqu. NaHCO₃ solution and the solvent evaporated to give 5-amino-3-chloro-2-cyanopyridine (4.34 g) as an off-white powder.

Step (f)

To a stirred solution of 5-amino-3-chloro-2-cyanopyridine (4.61 g) and NaOAc (4.81 g) in anhydrous AcOH (150 mL) at room temperature was added Br₂ (7.22 g). The solution was stirred at 60° C. for 2 h. Evaporation of the solvent and excess bromine gave crude 5-amino-6-bromo-3-chloro-2-cyano-pyridine (7.27 g). Recrystallization from AcOEt afforded clean product (6.23 g).

Step (g)

5-Amino-6-bromo-3-chloro-2-cyano-pyridine (1.6 g) was dissolved in THF (5 mL) at room temperature. N,N-dimethylaminopyridine (0.5 g) followed by Boc₂O (3.78 g) in small portions was added and the solution stirred at room temperature for 30 min to give after removal of the solvent 6-Bromo-5-(bis-carbamic acid tert-butyl ester)-3-chloro-2-cyanopyridine. The crude material was dissolved in dicloromethane (60 mL) and trifluoroacetic acid (1 g) added. The resulting solution was stirred for 1 h. The solvent was removed and the crude material purified by CC (AcOEt/hexane 1/1) to give 6-bromo-5-(tert-butoxycarbonylamino)-3-chloro-2-cyano-pyridine (1 g). MS (obs.): 333 (M+1).

### Reference 8

### Synthesis of 2-methoxymethylether-5-fluoro-phenylboronic acid

Step (a)

Commercially available 2-bromo-4-fluorophenol (25.0 g, 0.13 mol) was dissolved in 100 mL dry dichloromethane and 115 mL (1.30 mol) of dimethoxymethane. Phosphorus pentoxide (110.8 g, 0.39 mol) was added portion-wise, keeping the reaction temperature below 40° C. The reaction mixture was stirred vigorously at room temperature for 2 h, then carefully poured into 50 mL 1N aqueous NaOH. The organic layer was collected, washed with water and brine, dried over anhydrous MgSO₄, filtered and concentrated to give 2-methoxymethylether-5-fluorophenylbromide as a colorless oil (30.1 g, 100%).

Step (b)

A 500 mL round bottom flask was charged with a 1.6M solution of n-butyllithium in hexanes (100 mL, 0.16 mol), flushed with nitrogen and cooled to −78° C. A solution of 2-methoxymethylether-5-fluorophenylbromide (30.1 g, 0.13 mol) in 50 mL of dry THF was added dropwise over one hour. After one hour of stirring the reaction at −78° C., trimethylborate (20 mL, 0.175 mol) was added very slowly via syringe. The reaction was allowed to gradually warm to room temperature and after two hours the mixture was poured into ice, acidified to pH 4 with 5% aqueous citric acid and extracted with ethyl acetate (×3). The combined organic extracts were washed with water and brine, dried over MgSO₄ and filtered. Evaporation of the solvent under reduced pressure gave crude 2-methoxymethylether-5-fluorophenylboronic acid (28.2 g). Recrystallization from hexane afforded clean product (18.9 g, 73%). ¹H NMR (CDCl₃) δ ppm: 7.51 (d, J=2.1 Hz, 1H), 7.08 (m, 2H), 5.92 (bs, 2H), 5.25 (s, 2H), 3.51 (s, 3H).

### Example 1

### Synthesis of 2-[5-(5-carbamimidoyl-1H-benzoimidazol-2-yl)-6-hydroxy-3′-nitrobiphenyl-3-yl]succinic acid

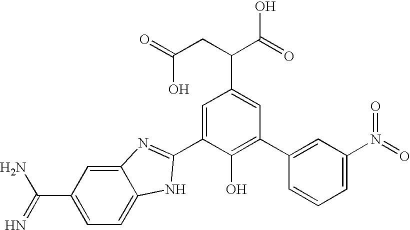

A mixture of 2-(5-formyl-6-hydroxy-3′-nitro-biphenyl-3-yl)-succinic acid (0.3 g, 0.835 mmol), prepared as in Reference 4, 3,4-diaminobenzamidine mono hydrochloride (0.17 g, 0.9 mmol) and benzoquinone (0.097 g, 0.9 mmol) in 50 mL of ethanol was heated for approximately 4 hours. The reaction mixture was cooled to ambient temperature and concentrated under reduced pressure. The residue was purified by reverse phase HPLC (gradient, acetonitrile/0.02 N aqueous HCl) to yield 2-[5-(5-carbamimidoyl-1H-benzoimidazol-2-yl)-6-hydroxy-3′-nitro-biphenyl-3-yl]-succinic acid (63% yield).

¹H NMR (DMSO-d₆) δ ppm: 2.67 (d of d, J=18 Hz, 6.0 Hz, 1H), 3.14 (d of d, J=18 Hz, 11.5 Hz, 1H), 3.97 (d of d, J=6.0 Hz, 11.5 Hz, 1H), 7.54 (d, J=2.1 Hz, 1H), 7.70-7.86 (m, 3H), 8.06 (d, J=8.8 Hz, 1H), 8.18-8.20 (m, 2H), 8.45 (t, J=2.1 Hz, 1H), 9.13 (br s, 2H), 9.40 (br s, 2H). MS: found (M+H) 490.4, (M−H) 488.4, calcd. 489.13.

Proceeding as in Example I and substituting suitable starting materials provided the following compounds of Formula I:

2-[3′-acetyl-5-(5-carbamimidoyl-1H-benzoimidazol-2-yl)-6-hydroxy-biphenyl-3-yl]-succinic acid; ¹H-NMR (d₆-DMSO) δ ppm: 9.40 (br s, 2H), 9.08 (br s, 2H), 8.16 (s, 1H), 8.13 (s, 2H), 7.91 (d, J=8.7 Hz, 1H), 7.82 (m, 2H), 7.70 (d, J=9.3 Hz, 1H), 7.57 (t, J=8.7 Hz, 1H), 7.45 (s, 1H), 3.94 (dd, J=10.3, 5.7 Hz, 1H), 3.12 (dd, J=15.7, 9.8 Hz, 1H), 2.58 (s, 3H,); MS LCMS Q⁺ 487.15 (calc.), 487.4 (obs.), Q⁻ 485.15 (calc.), 485.3 (obs);

2-[5-(5-carbamimidoyl-1H-benzoimidazol-2-yl)-3′-(1,1-difluoromethoxy)-6-hydroxy-biphenyl-3-yl]-succinic acid;

2-[5-(5-carbamimidoyl-1H-benzoimidazol-2-yl)-6,3′-dihydroxy-biphenyl-3-yl]-succinic acid; ¹H-NMR (d₆-DMSO) δ ppm: 9.36 (bs, 2H), 9.07 (bs, 2H), 8.15 (s, 1H), 8.06 (d, J=1.2, 1H), 7.81 (d, J=9.3 Hz, 1H), 7.71 (d, J=9.4 Hz, 1H), 7.36-7.25 (m, 4H), 7.33 (d, J=2.4 Hz, 1H), 7.18 (t, J=8.7 Hz, 1H), 6.98-6.94 (m, 2H), 6.73 (d, J=9.3 Hz, 1H), 3.91 (dd, J=10.2, 5.7 Hz, 1H), 3.10 (dd, J=19.6, 11.2 Hz, 1H), 2.69-2.60 (m, 1H); MS LCMS Q⁺ 461.14 (calc.), 461.7 (obs.), Q⁻ 459.14 (calc.), 459.3 (obs);

2-[5-(5-carbamimidoyl-1H-benzoimidazol-2-yl)-6,2′-dihydroxy-biphenyl-3-yl]-succinic acid; ¹H-NMR (d₆-DMSO) δ ppm: 9.48 (bs, 2H), 9.24 (bs, 2H), 8.23 (s, 1H), 8.15 (d, J=2.1 Hz, 1H), 7.88 (d, J=8.7 Hz, 1H), 7.79 (dd, J=8.7, 1.3 Hz, 1H), 7.32 (d, J=2.1 Hz, 1H), 7.19 (m, 2H), 6.98 (d, J=8.3 Hz, 1H), 6.87 (t, J=7.3 Hz, 1H), 3.94 (dd, J=10.3, 5.0 Hz, 1H), 3.14 (dd, J=16.9, 10.3 Hz, 1H), 2.69 (dd, J=16.9, 5.0 Hz, 1H): ¹³C NMR (d₆-DMSO) δ ppm: 174.04, 172.74, 165.96, 155.11, 154.79, 154.21, 134.27, 131.30, 128.99, 128.71, 128.10, 125.18, 124.45, 123.13, 122.46, 118.73, 115.74, 111.67, 46.09, 37.17: MS LCMS Q⁺ 461.146 (calc.), 461.8 (obs.), Q⁻ 459.130 (calc.), 459.4 (obs);

2-[5-(5-carbamimidoyl-1H-benzoimidazol-2-yl)-3′-aminocarbonyl-6-hydroxybiphenyl-3-yl]-succinic acid;

2-[5-(5-carbamimidoyl-1H-benzoimidazol-2-yl)-3′-cyano-6-hydroxy-biphenyl-3-yl]-succinic acid;

2-[5-(5-carbamimidoyl-1H-benzoimidazol-2-yl)-5′-fluoro-6,2′-dihydroxybiphenyl-3-yl]-succinic acid; ¹H-NMR (d₆-DMSO) δ ppm: 9.53 (bs, 2H), 9.29 (bs, 2H), 8.25 (d, J=1.0 Hz, 1H), 8.19 (d, J=2.1 Hz, 1H), 7.90 (d, J=8.5 Hz, 1H), 7.81 (dd, J=8.6, 1.5 Hz, 1H), 7.37 (d, J=2.1 Hz, 1H), 7.09-6.96 (m, 3H), 3.95 (dd, J=10.3, 4.7 Hz, 1H), 3.15 (dd, J=17.0, 10.3 Hz, 1H), 2.69 (dd, J=16.9, 5.0 Hz, 1H): MS LCMS Q⁺ 479.12 (calc.), 479.3 (obs.), Q⁻ 477.12 (calc.), 477.6 (obs);

2-[5-(5-carbamimidoyl-1H-benzoimidazol-2-yl)-5′-chloro-6,2′-dihydroxybiphenyl-3-yl]-succinic acid; ¹H NMR (DMSO-d6) δ 9.41 (s, 2H), 9.09 (s, 2H), 8.19 (s, 1H), 8.11 (s, 1H), 7.85 (d, 1H, J=8.5 Hz), 7.74 (d, 1H, J=8.5 Hz), 7.33 (s, 1H), 7.22 (m, 2H), 6.96 (d, 1H, J=8.3 Hz), 3.83 (m, 1H), 3.2-2.5 (m, overlapping with dmso and water peaks);

2-[5-(5-carbamimidoyl-6-fluoro-1H-benzoimidazol-2-yl)-6,2′-dihydroxybiphenyl-3-yl]-succinic acid;

2-[5-(5-carbamimidoyl-1H-benzoimidazol-2-yl)-6-hydroxy-2′-hydroxymethylbiphenyl-3-yl]-succinic acid;

2-[5-(5-carbamimidoyl-1H-benzoimidazol-2-yl)-5′-carboxy-6,2′-dihydroxybiphenyl-3-yl]-succinic acid;

2-[5-(5-carbamimidoyl-1H-benzoimidazol-2-yl)-6,2′,5′-trihydroxy-biphenyl-3-yl]-succinic acid;

2-[5-(5-carbamimidoyl-1H-benzoimidazol-2-yl)-6,2′,6′-trihydroxy-biphenyl-3-yl]-succinic acid; ¹H-NMR (d₆-DMSO) δ ppm: 9.45 (bs, 2H), 9.19 (bs, 2H), 8.21 (s, 1H), 8.07 (d, J=2.1 Hz, 1H), 7.87 (d, J=8.7 Hz, 1H), 7.77 (dd, J=8.5, 1.4 Hz, 1H), 7.16 (d, J=2.1 Hz, 1H), 6.94 (t, J=8.0 Hz, 1H), 6.40 (d, J=8.0 Hz, 2H), 3.91 (dd, J=10.6, 4.6 Hz, 1H), 3.12 (dd, J=17.1, 10.7 Hz, 1H), 2.65 (dd, J=17.0, 4.8 Hz, 1H): ¹³C NMR (d₆-DMSO) δ ppm: 174.10, 172.76, 165.96, 156.18, 155.73, 128.64, 125.04, 111.23, 106.37, 46.03, 37.30: MS LCMS Q⁺ 477.13 (calc.), 477.4 (obs.), Q⁻ 475.13 (calc.), 475.3 (obs);

2-[5-(5-carbamimidoyl-1H-benzoimidazol-2-yl)-6,2′-dihydroxy-5′-nitro-biphenyl-3-yl]-succinic acid;

2-[5-(5-carbamimidoyl-1H-benzoimidazol-2-yl)-2′-cyano-6-hydroxy-biphenyl-3-yl]-succinic acid;

2-[5-(6-carbamimidoyl-1H-benzoimidazol-2-yl)-6-hydroxy-3′-hydroxymethylbiphenyl-3-yl]-succinic acid; NMR (DMSO-d₆) δ ppm: 2.71 (d of d, J=6, 19 Hz, 1H), 3.16 (d of d, J=11, 19 Hz, 1H), 3.98 (d of d, J=6, 11 Hz, 1H), 4.57 (s, 2H), 7.32 (d, J=8 Hz), 7.42 (m, 3H), 7.50 (d of d, J=1, 8 Hz, 1H), 7.56 (d, J=1 Hz, 1H), 8.11 (d, J=2 Hz, 1H), 8.20 (br. s, 1H), 9.01 (s, 1H), 9.38 (s, 1H). LCMS: Calcd: 474.47; Obsd (MH+) 475.0; (MH−) 473.4.

2-[5-(5-carbamimidoyl-1H-benzoimidazol-2-yl)-5′-cyano-6,2′-dihydroxy-biphenyl-3-yl]-succinic acid;

2-[5-(5-carbamimidoyl-1H-benzoimidazol-2-yl)-5′-aminocarbonyl-6-hydroxy-2′-methoxy-biphenyl-3-yl]-succinic acid;

2-[5-(5-carbamimidoyl-1H-benzoimidazol-2-yl)-5′-cyano-6-hydroxy-2′-methoxy-biphenyl-3-yl]-succinic acid; and

2-[5-(5-carbamimidoyl-1H-benzoimidazol-2-yl)-5′-aminocarbonyl-6,2′-dihydroxybiphenyl-3-yl]-succinic acid.

### Example 2

### Synthesis of 2-[5-(5-carbamimidoyl-1H-indol-2-yl)-6-hydroxy-3′-nitro-biphenyl-3-yl]-succinic acid

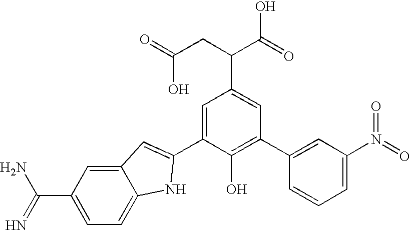

Step (a)

A mixture of 2-[5-ethynyl-6-(2-methoxyethoxymethoxy)-3′-nitro-biphenyl-3-yl]-succinic acid dimethyl ester (5.1 g, 10.8 mmol), prepared as in Reference 5, N-(4-cyano-2-iodo-phenyl)-methanesulfonamide (3.5 g, 10.8 mmol), triethylamine (15.1 mL, 108 mmol), Pd(Ph₃P)₂Cl₂ (0.154 g, 0.22 mmol) and acetonitrile (150 mL) was agitated by bubbling with nitrogen gas for approximately 5 minutes and then combined with copper(I)iodide (0.041 g, 0.22 mmol). This mixture was heated to reflux for about 1 hour, then cooled to ambient temperature and mixed with 5% citric acid. This mixture was extracted with DCM and the DCM layer was dried (MgSO₄) and concentrated under reduced pressure. The residue was purified by column chromatography on silica (eluent, hexane/ethyl acetate) to yield 2-[5-(5-cyano-1-methanesulfonyl-1H-indol-2-yl)-6-(2-methoxy-ethoxymethoxy)-3′-nitro-biphenyl-3-yl]-succinic acid dimethyl ester (41% yield).

Step (b)

A mixture of 2-[5-(5-cyano-1-methanesulfonyl-1H-indol-2-yl)-6-(2-methoxy-ethoxymethoxy)-3′-nitro-biphenyl-3-yl]-succinic acid dimethyl ester (2.93 g, 4.4 mmol), methanol (90 mL) and 50% aqueous NaOH (30 mL) was agitated at 50° C. for approximately 2 hours. The mixture was cooled to ambient temperature and combined with 10% aqueous citric acid (excess). This mixture was extracted with ethyl acetate (×3) and the combined extracts were sequentially washed with water and brine, dried (MgSO₄) and concentrated under reduced pressure to afford 2-[5-(5-cyano-1H-indol-2-yl)-6-(2-methoxyethoxymethoxy)-3′-nitro-biphenyl-3-yl]-succinic acid (99% yield). MS: found (M+H) 559.4, calc 559.16.

Step (c)

A mixture of 2-[5-(5-cyano-1H-indol-2-yl)-6-(2-methoxy-ethoxymethoxy)-3′-nitro-biphenyl-3-yl]-succinic acid (2.5 g, 4.4 mmol), dry methanol (20 mL) and 4N HCl in dioxane (20 mL) was agitated at ambient temperature for approximately 2 hours. The reaction mixture was concentrated under reduced pressure and the residue was purified by silica gel column chromatography (eluent, 30% EtOAc in hexane) to afford 2-[5-(5-cyano-1H-indol-2-yl)-6-hydroxy-3′-nitro-biphenyl-3-yl]-succinic acid dimethyl ester (64% yield).

MS: found (M+H) 500.4, (M−H) 498.4, calc 499.14.

Step (d)

A mixture of 2-[5-(5-cyano-1H-indol-2-yl)-6-hydroxy-3′-nitro-biphenyl-3-yl]-succinic acid dimethyl ester (1.3 g, 2.6 mmol) and dry methanol was cooled to about 0° C. and then bubbled with dry HCl gas. This mixture was sealed in a reaction vessel, agitated at ambient temperature for approximately 24 hours and then bubbled with nitrogen gas. This mixture was concentrated under reduced pressure to afford 2-[6-hydroxy-5-(5-methoxycarbonimidoyl-1H-indol-2-yl)-3′-nitro-biphenyl-3-yl]-succinic acid dimethyl ester.

Step (e)

A mixture of 2-[6-hydroxy-5-(5-methoxycarbonimidoyl-1H-indol-2-yl)-3′-nitrobiphenyl-3-yl]-succinic acid dimethyl ester (1.5 g, 2.6 mmol) and methanol was combined with crystalline ammonium carbonate (excess) added in portions. This mixture then was agitated at ambient temperature for approximately 8 hours and then concentrated under reduced pressure. The residue was treated with aqueous 1N HCl forming a precipitate. The precipitate was isolated, washed with a minimum amount of 1N HCl and dried over P₂O₅ under high vaccum to yield 2-[5-(5-carbamimidoyl-1H-indol-2-yl)-6-hydroxy-3′-nitro-biphenyl-3-yl]-succinic acid dimethyl ester (70% yield). MS: found (M+H) 517.3, calc 516.16.

Step (f)

A mixture of 2-[5-(5-carbamimidoyl-1H-indol-2-yl)-6-hydroxy-3′-nitro-biphenyl-3-yl]-succinic acid dimethyl ester (1 g, 1.8 mmol), 3.5 N aqueous HCl (60 mL) and acetonitrile (20 mL) was agitated at reflux conditions for approximately 1 hour. The mixture was cooled to ambient temperature and concentrated under reduced pressure. The residue was purified by reverse phase HPLC (acetonitrile/0.02 N HCl gradient) to afford 2-[5-(5-carbamimidoyl-1H-indol-2-yl)-6-hydroxy-3′-nitro-biphenyl-3-yl]-succinic acid (33% yield).

¹H NMR (DMSO-d₆) δ ppm: 2.69 (d of d, J=6.0 Hz, 18.1 Hz, 1H), 3.25 (d of d, J=18.1 Hz, 9.8 Hz, 1H), 3.98 (d of d, J=6.0 Hz, 9.8 Hz, 1H), 7.17 (s, 1H), 7.28-8.40 (m, 9H) 8.84 (br s, 2H), 9.19 (br s, 2H), 9.33 (s, 1H). MS: found (M+H) 489.2, calc 488.13.

Proceeding as described in Example 2 above but starting with suitable starting material provided the following compounds of Formula I:

2-[5-(5-carbamimidoyl-1H-indol-2-yl)-6,2′-dihydroxy-biphenyl-3-yl]-succinic acid; and

2-[5-(5-carbamimidoyl-6-chloro-1H-indol-2-yl)-6,2′-dihydroxy-biphenyl-3-yl]-succinic acid.

### Example 3

### Synthesis of (E)-2-[5-(5-carbamimidoyl-1H-indol-2-yl)-5′-fluoro-6,2′-dihydroxybiphenyl-3-yl]-but-2-enedioic acid and (Z)-2-[5-(5-Carbamimidoyl-1H-indol-2-yl)-5′-fluoro-6,2′-dihydroxybiphenyl-3-yl]-but-2-enedioic acid

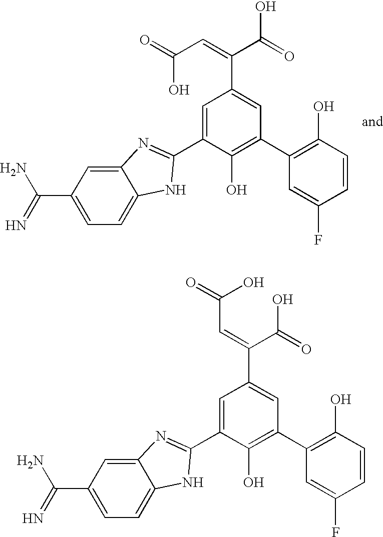

Step (a)

A 200 mL 24/40 round bottom flask was charged with (E)-2-(5′-fluoro-5-formyl-6,2′-dihydroxy-biphenyl-3-yl)-but-2-enedioic acid dimethyl ester (100 mg, 0.27 mmol), prepared as in Reference 6, 3,4-diamino-benzamidine hydrochloride (55 mg, 0.29 mmol), methanol (30 mL) and a magnetic stir bar. The reaction mixture was stirred and heated to reflux for 48 hours and then concentrated via rotoevaportion to give a mixture of (Z)-2-[5-(5-carbamimidoyl-1H-benzoimidazol-2-yl)-5′-fluoro-6,2′-dihydroxy-biphenyl-3-yl]-but-2-enedioic acid dimethyl ester and (Z)-2-[5-(5-carbamimidoyl-1H-benzoimidazol-2-yl)-5′-fluoro-6,2′-dihydroxy-biphenyl-3-yl]-but-2-enedioic acid dimethyl ester as a dark gum. MS LCMS Q⁻ 503.145 (calc.), 503.3 (obs.) Q⁺ 505.152 (calc.), 505.3 (obs.).

Step (b)

A 250 mL round bottom flask was charged with the mixture of (Z)-2-[5-(5-carbamimidoyl-1H-benzoimidazol-2-yl)-5′-fluoro-6,2′-dihydroxy-biphenyl-3-yl]-but-2-enedioic acid dimethyl ester and (Z)-2-[5-(5-carbamimidoyl-1H-benzoimidazol-2-yl)-5′-fluoro-6,2′-dihydroxy-biphenyl-3-yl]-but-2-enedioic acid dimethyl ester (70 mg, 0.14 mmol), 1 N hydrochloric acid (20 mL) and a magnetic stir bar. The reaction mixture was stirred and heated to reflux for 4 hours and then concentrated to a solid via rotoevaporation. The residue was dissolved in 20% CH₃CN, 80% 20 mM HCl (10 mL) the individual isomers were purified from the solution using reverse phase C-18 HPLC and then lyophilized to give:

(E)-2-[5-(5-carbamimidoyl-1H-indol-2-yl)-5′-fluoro-6,2′-dihydroxy-biphenyl-3-yl]-but-2-enedioic acid MS LCMS Q⁻ 475.113 (calc.), Q⁺ 477.4 (calc.), 477.121 (obs.); ¹H-NMR (d₆-DMSO) δ ppm: 9.34 (s, 2H), 8.88 (s, 2H), 8.18 (d, J=2.23 Hz, 1H), 8.06 (d, J=1.98 Hz, 1H), 7.86 (d, J=7.92 Hz, 1H), 7.73 (dd, J=8.16, 1.98 Hz, 1H), 7.29 (d, J=2.23 Hz, 1H), 7.02 (m, 2H), 6.93 (s, 2H); and

(Z)-2-[5-(5-carbamimidoyl-1H-indol-2-yl)-5′-fluoro-6,2′-dihydroxy-biphenyl-3-yl]-but-2-enedioic acid; MS LCMS Q⁻ 475.113 (calc.), Q⁺ 477.121 (calc.), 477.2 (obs.);

HPLC (C-18 reverse phase) 6.52 min (1-90); ¹H-NMR (d₆-DMSO) δ ppm: 9.35 (s, 2H), 8.88 (s, 1H), 8.49 (d, J=1.24 Hz, 1H), 7.90 (d, J=8.66 Hz, 1H), 7.76 (dd, J=8.97, 1.65 Hz, 1H), 7.47 (d, J=1.65 Hz, 1H), 7.07 (s, 2H), 6.93 (m, 1H), 6.46 (s, 1H).

### Example 4

### Synthesis of 2-[5-(5-carbamimidoyl-1H-benzoimidazol-2-yl)-5′-cyano-6,2′-dihydroxy-biphenyl-3-yl]-succinic acid

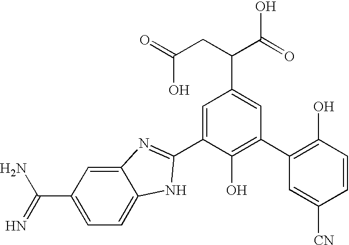

Step (a)

In a 500 mL 24/40 round bottom flask, a solution of 2-(3-bromo-5-formyl-4-hydroxy-phenyl)-succinic acid dimethyl ester (6.0 g, 13.8 mmol) (prepared as described in Reference 2 and Reference 5, Step (a), above), dioxane (120 mL), bis(pinacolato)diboron (4.3 g, 16.6 mmol) and potassium acetate (4.1 g, 41.4 mmol) were combined. Nitrogen was bubbled through the solution and then dichloro[1,1′bis(diphenylphosphino)ferrocene] palladium(II)dichloromethane adduct (purchased from STREM cat# 46-0450) (0.56 g, 0.7 mmol) was added, the solution was refluxed for 3 hours. After cooling, the solution was taken up in EtOAc (50 mL), washed with 5% citric acid, brine, and dried. The solvent is removed under reduced pressure. The residue was taken up in 138 mL toluene to generate a 0.1 M solution of 2-[3-formyl-4-(2-methoxy-ethoxymethoxy)-5-(4,4,5,5-tetramethyl-[1,3,2]dioxa-borolan-2-yl)-phenyl]-succinic acid dimethyl ester.

Step (b)

To a mixture of 3-bromo-4-hydroxy-benzonitrile (1.0 g, 5.3 mmol), Hunig's base (0.82 g, 6.4 mmol), and dichloromethane (30 mL), 1-chloromethoxy-2-methoxyethane (0.72 g, 5.8 mmol) was added slowly to the mixture and allowed to stir for 3 hours. The solution was extracted with H₂O (2×50 mL), the organic layer was dried over MgSO₄ and concentrated under reduced pressure to give crude 3-bromo-4-(2-methoxyethoxymethoxy)benzonitrile.

Step (c)

A solution of 2-[3-formyl-4-(2-methoxy-ethoxymethoxy)-5-(4,4,5,5-tetramethyl-[1,3,2]dioxa-borolan-2-yl)-phenyl]-succinic acid dimethyl ester (47 mL of a 0.1M solution), prepared as described in Step (a) above, was added to 3-bromo-4-(2-methoxyethoxymethoxy)benzonitrile, followed by addition of 2M Na₂CO₃ (50 mL) and Pd(PPh₃)₄ (0.27 g, 0.2 mmol). The reaction mixture was refluxed for 12 hours, cooled and extracted with EtOAc (2×50 mL) and H₂O (2×50 mL). The organic layer was dried over MgSO₄, concentrated under reduced pressure and the residue taken up in MeOH (30 mL). To this solution, 3,4-diaminobenzamidine monohydrochloride (0.87 g, 4.7 mmol) was added and the reaction mixture was refluxed overnight, cooled, the solvent was removed under pressure. 1N HCl was added (30 mL) and the solution was refluxed for 3 hours, cooled and again the solvent was removed under reduced pressure. The residue was taken up in 8 mL 0.1 M HCl and purified by reverse phase HPLC (gradient, acetonitrile/0.02 N aqueous HCl) to yield 250 mg (0.5 mmol, 9%) of 2-[5-(5-carbamimidoyl-1H-benzoimidazol-2-yl)-5′-cyano-6,2′-dihydroxy-biphenyl-3-yl]-succinic acid as the HCl salt. ¹H-NMR (d₆-DMSO) δ ppm: 9.48 (bs, 2H), 9.23 (bs, 2H), 8.23 (d, J=0.8 Hz, 1H), 8.19 (d, J=1.9 Hz, 1H), 7.88 (d, J=8.7 Hz, 1H), 7.79 (dd, J=8.7, 1.4 Hz, 1H), 7.70-7.64 (m, 2H), 7.35 (d, J=1.9 Hz, 1H), 7.18 (d, J=8.0 Hz, 1H), 3.95 (dd, J=10.3, 4.7 Hz, 1H), 3.15 (dd, J=16.9, 10.3 Hz, 1H), 2.69 (dd, J=16.9, 5.0 Hz, 1H): MS LCMS Q⁺ 486.14 (calc.), 486.2 (obs.), Q⁻ 484.13 (calc.), 484.2 (obs).

Following the procedure described in Example 4 above, but substituting 3-bromo-4-hydroxybenzonitrile with 3-bromobenzenesulfonamide gave 2-[3′-aminosulfonyl-5-(5-carbamimidoyl-1H-benzoimidazol-2-yl)-6-hydroxybiphenyl-3-yl]-succinic acid.

¹H-NMR (d₆-DMSO) δ ppm: 9.38 (bs, 2H), 9.07 (bs, 2H), 8.176 (bs, 2H), 8.08 (s, 1H), 7.80-7.85 (m, 3H), 7.74 (d, J=8.2 Hz, 1H), 7.65 (t, J=7.8 Hz, 1H), 7.46 (s, 1H), 7.41 (bs, 2H), 3.99 (dd, J=9.8, 5.1 Hz, 1H), 3.15 (dd, J=16.8, 10.1 Hz, 1H), 2.72 (dd, J=16.8, 5.1 Hz, 1H): MS LCMS Q⁺ 523.12 (calc.), 524.6 (obs.).

### Example 5

### Synthesis of 2-[5′-aminocarbonyl-5-(5-carbamimidoyl-1H-benzoimidazol-2-yl)-6,2′-dihydroxy-biphenyl-3-yl]-succinic acid

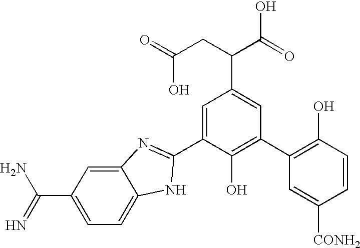

2-[5-(5-Carbamimidoyl-1H-benzoimidazol-2-yl)-5′-cyano-6,2′-dihydroxy-biphenyl-3-yl]-succinic acid) (50 mgs, 0.1 mmol), prepared as described in Example 4 above, was dissolved in conc. HCl (20 mL) and heated at 60° C. for 3 days. The solvent was reduced under pressure and the residue was taken up in 0.1 M HCl (8 mL) and purified by reverse phase HPLC (gradient, acetonitrile/0.02 N aqueous HCl) to yield (17 mg, 0.03 mmol 30%) of 2-[5′-aminocarbonyl-5-(5-carbamimidoyl-1H-benzoimidazol-2-yl)-6,2′-dihydroxy-biphenyl-3-yl]-succinic acid as the HCl salt. ¹H-NMR (d₆-DMSO) δ ppm: 9.43 (bs, 2H), 9.16 (bs, 2H), 8.21 (d, J=0.9 Hz, 1H), 8.14 (d, J=2.1 Hz, 1H), 7.87 (d, J=8.7 Hz, 1H), 7.79-7.73 (m, 3H), 7.32 (d, J=2.1 Hz, 1H), 7.00-6.95 (m, 1H), 3.95 (dd, J=10.3, 4.7 Hz, 1H), 3.15 (dd, J=17.0, 10.3 Hz, 1H), 2.69 (dd, J=17.0, 5.0 Hz, 1H): MS LCMS Q⁺ 504.15 (calc.), 504.5 (obs.), Q⁻ 502.14 (calc.), 502.5 (obs).

### Example 6

### Synthesis of 2-[5′-aminomethyl-5-(5-carbamimidoyl-1H-benzoimidazol-2-yl)-6,2′-dihydroxy-biphenyl-3-yl]-succinic acid

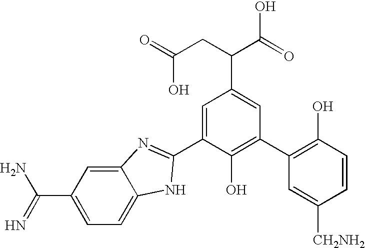

2-[5-(5-Carbamimidoyl-1H-benzoimidazol-2-yl)-5′-cyano-6,2′-dihydroxy-biphenyl-3-yl]-succinic acid) (120 mgs, 0.23 mmol) was dissolved in MeOH (30 mL) and Pearlman's catalyst (10% Pd(OH)₂ on activated carbon; 20 mgs) was added and stirred under one atmosphere of hydrogen for 5 hours. The solution was filtered, the solvent removed under reduced pressure, and the residue was taken up in 0.1 M HCl (8 mL) and purified by reverse phase HPLC (gradient, acetonitrile/0.02 N aqueous HCl_{conc}. to yield 35 mg (27%) of 2-[5′-aminomethyl-5-(5-carbamimidoyl-1H-benzoimidazol-2-yl)-6,2′-dihydroxy-biphenyl-3-yl]-succinic acid as the bis HCl salt. ¹H-NMR (d₆-DMSO) δ ppm: 9.51 (bs, 2H), 9.28 (bs, 2H), 8.37 (bs, 3H), 8.24 (s, 1H), 8.19 (d, J=2.1 Hz, 1H), 7.87 (d, J=8.5 Hz, 1H), 7.79 (dd, J=8.6, 1.4 Hz, 1H), 7.36-7.31 (m, 2H), 7.30 (d, J=2.1 Hz, 1H), 7.01 (d, J=8.9 Hz, 1H), 3.94 (m, 3H), 3.14 (dd, J=16.9, 10.3 Hz, 1H), 2.69 (dd, J=16.9, 5.0 Hz, 1H): ¹³C NMR (d₆-DMSO) δ ppm: 174.03, 172.72, 166.01, 155.19, 128.98, 124.02, 115.74, 111.75, 41.87, 37.22; MS LCMS Q⁺ 490.17 (calc.), 490.4 (obs.), Q⁻ 488.16 (calc.), 488.4 (obs).

### Example 7

### Synthesis of 2-[5-(5-carbamimidoyl-1H-benzoimidazol-2-yl)-6,2′-dihydroxy-5′-ureidomethyl-biphenyl-3-yl]-succinic acid

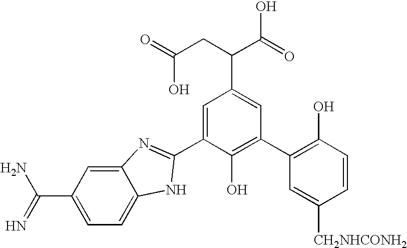

2-[5′-Aminomethyl-5-(5-carbamimidoyl-1H-benzoimidazol-2-yl)-6,2′-dihydroxybiphenyl-3-yl]-succinic acid) (20 mgs,), prepared as described in Example 5 above, was dissolved in MeOH and potassium cyanate (100 mg) was added portionwise and the mixture was stirred for 3 days. The residue was taken up in 0.1 N HCl (8 mL) and purified by reverse phase HPLC (gradient, acetonitrile/0.02 N aqueous HCl) to yield 4 mg (20%) of 2-[5-(5-Carbamimidoyl-H-benzoimidazol-2-yl)-6,2′-dihydroxy-5′-ureidomethyl-biphenyl-3-yl]-succinic acid) as the HCl salt. ¹H-NMR (400 MHz) (d₆-DMSO) δ ppm: 9.47 (bs, 2H), 9.21 (bs, 2H), 8.21 (d, J=0.8 Hz, 1H), 8.12 (d, J=2.0 Hz, 1H), 7.87 (d, J=8.4 Hz, 1H), 7.78 (dd, J=8.60, 1.4 Hz, 1H), 7.29 (d, J=2.0 Hz, 1H), 7.11-7.07 (m, 2H), 6.91 (d, J=8.8 Hz, 1H), 4.14 (s, 2H), 3.93 (dd, J=10.6, 5.0 Hz, 1H), 3.13 (dd, J=16.8, 10.0 Hz, 1H), 2.68 (dd, J=16.8, 4.8 Hz, 1H): MS LCMS Q⁺ 533.18 (calc.), 533.4 (obs.), Q⁻ 531.16 (calc.), 531.4 (obs).

### Example 8

### Synthesis of 2-[5′-amino-5-(5-carbamimidoyl-1H-benzoimidazol-2-yl)-6,2′-dihydroxy-biphenyl-3-yl]-succinic acid

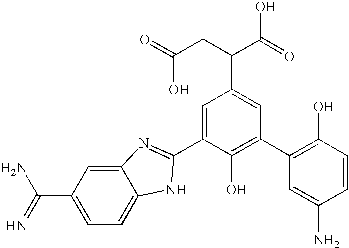

Step (a)

2-[5-(5-carbamimidoyl-1H-benzimidazol-2-yl)-2′-methoxy-5′-nitrobiphenyl-3-yl]succinic acid dimethyl ester (0. 14 g, 0.25 mmol), prepared as described in Example 4, Step (b) but substituting 3-bromo-4-(2-methoxyethoxymethoxy)benzonitrile with 2-bromo-4-nitroanisole, was dissolved in 8 mL of HBr (48% aq) and the reaction mixture heated at 100° C. over 9 hrs. The resulting product was purified by reverse phase HPLC to afford 63 mg (50%) 2-[5-(5-carbamimidoyl-1H-benzoimidazol-2-yl)-6,2′-dihydroxy-5′-nitrobiphenyl-3-yl]-succinic acid as a brown solid. LCMS found (M+1) 506.2, calcd. 505.44.

Step (b)

2-[5′amino-5-(5-carbamimidoyl-1H-benzoimidazol-2-yl)-6,2′-dihydroxybiphenyl-3-yl]-succinic acid (0.2 g, 0.39 mmol) was dissolved in MeOH (3 mL) and saturated aq. NH₄Cl (4 mL). Iron powder (400 mg) was added and the mixture heated to reflux for 2 hr with monitoring until it went to completion. Aq 1N HCl (5 mL) and additional MeOH (5 mL) was added and the mixture filtered to remove the metal. The crude filtrate was purified by reverse phase HPLC to afford 40 mg (21%) of 2-[5′-amino-5-(5-carbamimidoyl-1H-benzoimidazol-2-yl)-6,2′-dihydroxy-biphenyl-3-yl]-succinic acid as a brown solid. LCMS found (M+1) 476.3, calcd. 475.46.

### Example 9

### Synthesis of 2-[5-(5-carbamimidoyl-1H-indol-2-yl)-2′,6-dihydroxy-5′-aminocarbonyl-biphenyl-3-yl]-succinic acid

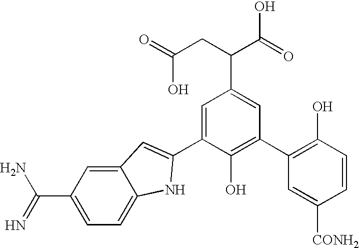

Step (a)

3-Bromo-2-(methoxyethoxymethoxy)benzonitrile (1.28 gm, 4.5 mmol) (prepared from 3-bromo-2-hydroxybenzonitrile as described in Reference 5, Step (a) above) was dissolved in MeOH (10 mL) and was treated with NaBO₃ (18 mmol) in water (5 mL) and the reaction mixture heated at 50° C. for 7 hrs. Workup involved extraction of the product into ethyl acetate and drying to afford 4-aminocarbonyl-2-(methoxyethoxymethoxy)-bromobenzene 1.32 g (96%).

Step (b)

4-Aminocarbonyl-2-(methoxyethoxymethoxy)bromobenzene was condensed with 2-[3-formyl-4-(2-methoxyethoxymethoxy)-5-(4,4,5,5-tetramethyl-[1,3,2]dioxa-borolan-2-yl)-phenyl]-succinic acid dimethyl ester and then converted to 2-[5-ethynyl-6-(2-methoxyethoxymethoxy)-5′-aminocarbonyl-2′-(methoxyethoxymethoxy)-biphenyl-3-yl]succinic acid dimethyl ester as described previously.

Step (c)

2-[5-Ethynyl-6-(2-methoxyethoxymethoxy)-5′-aminocarbonyl-2′-(methoxyethoxymethoxy)-biphenyl-3-yl]succinic acid dimethyl ester (1.6 gm, 2.8 mmol), 3-iodo-4-(N-tert-butoxycarbonylamino)benzonitrile (0.91 gm, 2.7 mmol) were dissolved in acetonitrile (15 mL) and triethylamine (4 mL, 28 mmol) and N₂ bubbled through the mixture for 5 min. bistriphenyphosphine palladium (II) chloride (0.08 gm, 0.11 mmol) was added and the reaction mixture was heated under a N₂ atmosphere. Copper iodide (0.01 gm, 0.05 mmol) was added after 5 min. and reflux was continued for 1 hr. The reaction was worked up with citric acid (5% aq., 20 mL) and ethylacetate and subsequently, the organic layer was washed with water and brine, dried over MgSO4 and concentrated. The crude product thus obtained was purified by flash chromatography (ethyl acetate) to afford the desired product 2-{5-[2-(5-cyano-2-(tert-butoxycarbonylamino)phenyl)ethynyl]-6-(2-methoxyethoxymethoxy)-5′-aminocarbonyl-2′-(methoxyethoxymethoxy)-biphenyl-3-yl]succinic acid dimethyl ester (1.7 gm, 79%) as a foam. The identity of this compound was confirmed by LCMS calcd 789.31, found (−ve) 789.0, (+ve) 812.6 (M+Na).

Step (d)

A solution of 2-{5-[2-(5-cyano-2-(tert-butoxycarbonylamino)phenyl)ethynyl]-6-(2-methoxyethoxymethoxy)-5′-aminocarbonyl-2′-(methoxyethoxymethoxy)-biphenyl-3-yl]succinic acid dimethyl ester (1.7 gm, 2.15 mmol) in THF (20 mL) was treated with tetrabutylammonium fluoride (1M, 4.5 mL) and the mixture stirred for 4 hrs. at room temperature. Workup involved adding citric acid (5% aq, 10 mL) and ethyl acetate (20 mL), washing the organic layer with water, brine and drying over MgSO4 and concentrating to obtain the crude mixture. The mixture obtained was taken as is, dissolved in anh. MeOH (10 mL), HCl(anh.), (4 M in dioxane, 10 mL) was added and the reaction mixture stirred at room temperature over 9 hrs. Upon drying (high vacuum) 2-[5-(5-cyano-1-(tert-butoxycarbonylamino)-1H-indol-2-yl]-2′,6-dihydroxy-5′-aminocarbonyl-biphenyl-3-yl]-succinic acid dimethyl ester was isolated which was then converted to 2-[5-(5-carbamimidoyl-1H-indol-2-yl)-2′,6-dihydroxy-5′-aminocarbonyl-biphenyl-3-yl]-succinic acid as described in Example 2 above.

1H NMR (DMSO-d₆) δ ppm: 10.49 (s, 1H), 9.18 (s, 2H), 8.82 (s, 2H), 8.65 (s, 1H), 8.11 (s, 1H), 7.72 (m, 5H), 7.12 (m, 3H), 3.91 (m, 1H), 3.15 (br dd, overlapping with water peak), 2.59 (m, overlapping with DMSO peak). LCMS found (M+1) 503.5, calcd. 502.48.

### Example 10

### Synthesis of 1-ethyl-2-[5-(5-carbamimidoyl-1H-benzimidazol-2-yl)-2′,6-dihydroxy-5′-fluorobiphenyl-3-yl]-succinate and 4-ethyl-2-[5-(5-carbamimidoyl-1H-benzimidazol-2-yl)-2′,6-dihydroxy-5′-fluorobiphenyl-3-yl]-succinate

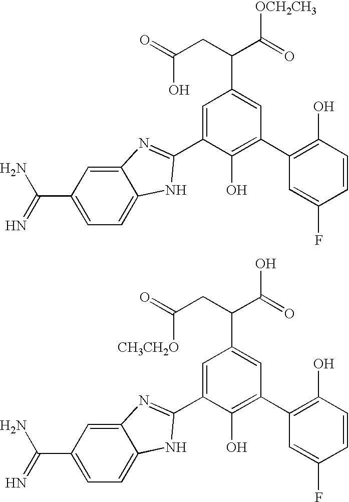

A 250 mL 24/40 round bottom flask was charged with the mono-hydrochloride salt of 2-[5-(5-carbamimidoyl-1H-benzimidazol-2-yl)-2′,6-dihydroxy-5′-fluorobiphenyl-3-yl]-succinic acid (0.10 g, 0.194 mol), anhydrous ethanol (50 mL), and a magnetic stir bar. The above reaction mixture was heated until reflux until all starting material was consumed. The principle component of the reaction mixture was 4-ethyl-2-[5-(5-carbamimidoyl-1H-benzimidazol-2-yl)-2′,6-dihydroxy-5′-fluorobiphenyl-3-yl]-succinate with lesser amounts of 1-ethyl-2-[5-(5-carbamimidoyl-1H-benzimidazol-2-yl)-2′,6-dihydroxy-5′-fluorobiphenyl-3-yl]-succinate and the diethyl ester. The reaction mixture was rotovaped to dryness. The solids were dissolved in a solution containing 4:1 20 mM HCl:acetonitrile and separated on a preparative HPLC system utilizing a standard C₁₈ column to give the 1-ethyl-2-[5-(5-carbamimidoyl-1H-benzimidazol-2-yl)-2′,6-dihydroxy-5′-fluorobiphenyl-3-yl]-succinate, followed by 4-ethyl-2-[5-(5-carbamimidoyl-1H-benzimidazol-2-yl)-2′,6-dihydroxy-5′-fluorobiphenyl-3-yl]-succinate and finally the diethyl ester. The appropriate pure fractions were combined and lyophilized to yield the above compound with >97% purity.

Alternate Method:

A 250 mL 24/40 round bottom flask was charged with the mono-hydrochloride salt of 1,4-diethyl-2-[5-(5-carbamimidoyl-1H-benzimidazol-2-yl)-2′,6-dihydroxy-5′-fluorobiphenyl-3-yl]-succinate (0.10 g, 0.175 mmol), 1N aqueous HCl (50 mL), and a magnetic stir bar. The above reaction mixture was heated at 50° C. until all starting material was consumed. The principle component of the reaction mixture was 1-ethyl-2-[5-(5-carbamimidoyl-1H-benzimidazol-2-yl)-2′,6-dihydroxy-5′-fluorobiphenyl-3-yl]-succinate with lesser amounts of 4-ethyl-2-[5-(5-carbamimidoyl-1H-benzimidazol-2-yl)-2′,6-dihydroxy-5′-fluorobiphenyl-3-yl]-succinate and additionally the diacid. The reaction mixture was lyophilized to dryness. The solids were dissolved in a solution containing 4:1 20 mM HCl:acetonitrile and separated on a preparative HPLC system utilizing a standard C₁₈ column. The order of elution of compounds was 2-[5-(5-carbamimidoyl-1H-benzimidazol-2-yl)-2′,6-dihydroxy-5′-fluorobiphenyl-3-yl]-succinic acid, 1-ethyl-2-[5-(5-carbamimidoyl-1H-benzimidazol-2-yl)-2′,6-dihydroxy-5′-fluorobiphenyl-3-yl]-succinate and 4-ethyl-2-[5-(5-carbamimidoyl-1H-benzimidazol-2-yl)-2′,6-dihydroxy-5′-fluorobiphenyl-3-yl]-succinate. The appropriate pure fractions were combined and lyophilized to yield, in >97% purity, of the desired compounds.

Ethyl-2-[5-(5-carbamimidoyl-1H-benzimidazol-2-yl)-2′,6-dihydroxy-5′-fluorobiphenyl-3-yl]-succinate. ¹H NMR (d₆-DMSO) δ ppm: 9.36 (bs, 2H), 9.00 (bs, 2H), 8.18 (s, 1H), 8.08 (s, 1H), 7.85 (d, J=8.4 Hz, 1H), 7.73 (d, J=9.6 Hz, 1H), 7.34 (s, 1H), 7.02 (m, 1H), 6.92 (m, 1H), 6.78 (bs, 1H), 4.08 (m, 2H), 4.00 (dd, J=10.4 Hz, 4.8 Hz, 1H), 3.15 (dd, J=16.8 Hz, 11.2 Hz, 1H), 2.72 (dd, J=8.4 Hz, 4.8 Hz, 1H), 1.14 (t, J=14.0 Hz, 3H).

4-Ethyl-2-[5-(5-carbamimidoyl-1H-benzimidazol-2-yl)-2′,6-dihydroxy-5′-fluorobiphenyl-3-yl]-succinate. ¹H NMR (d₆-DMSO) δ ppm: 9.39 (bs, 2H), 9.09 (bs, 2H), 8.19 (s, 1H), 8.12 (s, 1H), 7.82 (d, J=8.1 Hz, 1H), 7.76 (d, J=8.1 Hz, 1H), 7.36 (s, 1H), 7.02 (m, 1H), 6.93 (m, 1H), 6.80 (bs, 1H), 4.07 (dd, J=7.6 Hz, 2.8 Hz, 2H), 3.97 (dd, J=10.4 Hz, 5.6 Hz, 1H), 3.19 (d, J=8.1 Hz, 1H), 2.76 (d, J=8.1 Hz, 1H), 1.18 (t, J=14.0 Hz, 3H).

### Example 11

### Synthesis of 2-[5-(5-carbamimidoyl-1H-benzimidazol-2-yl)-5′-(2-cyanoethyl)-2′,6-dihydroxybiphenyl-3-yl]-succinic acid

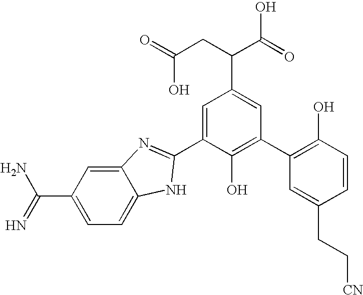

Commercially available 3-(4-hydroxyphenyl)propionitrile (5.0 g, 0.034 mol) was dissolved in DMF (50 mL) and cooled to 0° C. Bromine (5.4 g, 1.7 mL, 0.034 mol) was added slowly and the reaction was warmed to room temp over 2 hours. Triethylamine (10 mL) and MEMCl (7 mL) were added and stirred for 2 hours. To this mixture was added 5% NaOH (50 mL) and the solution was extracted with EtOAc (2×100 mL). The organic layer was dried with MgSO4 and the solvent was removed under reduced pressure. The residue was columned on SiO₂ with 10% EtOAc/hexanes as the eluent to give 0.64 g of 3-[3-bromo-4-(2-methoxyethoxymethoxy)phenyl]-propionitrile as an oil which was converted to the title compound by following the procedure described in Example 4 above.

1H NMR (d₆-DMSO) δ ppm: 9.29 (s, 2H), 8.91 (s, 2H), 8.11 (s, 1 H), 8.00 (d, 1 H, J=7 Hz), 7.79 (d, 1 H, J=8 Hz), 7.67 (d, 1 H, J=5 Hz), 7.24 (d, 1 H, J=2 Hz), 7.06 (s, 1 H), 7.04 (d, 1 H, J=2 Hz), 6.81 (d, 1 H, J=9 Hz), 3.85 (m, 1 H), 3.07 (dd, 1H, J=11, 15 Hz), 2.44 (M, 4 H), 2.75-2.58 (m, 1 H). MS LCMS Q⁺ 514.16 (calc.), 514.6 (obs).

### Example 12

### Synthesis of 2-[5-(5-carbamimidoyl-1H-benzimidazol-2-yl)-3′-bromo-5′-(2-cyanomethyl)-2′,6-dihydroxybiphenyl-3-yl]-succinic acid

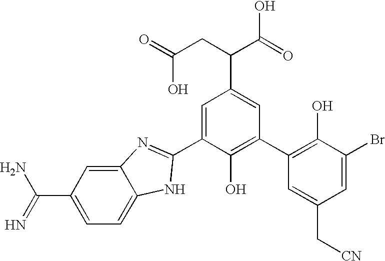

3-Bromo-4-(2-methoxyethoxymethoxy)-phenyl]acetonitrile was prepared in the identical manner as described in Example 11 above, but substituting 3-(4-hydroxyphenyl)-propionitrile with 4-hydroxyphenyl-acetonitrile (5.0 g, 0.037 mol) as the starting material.

1H NMR (d₆-DMSO) δ ppm: 7.67 (s, 1 H), 5.18 (s, 1 H), 4.02 (s, 1 H), 3.96-3.93 (m, 1 H), 3.51-3.47 (m, 1 H<), 3.23 (s, 2 H), 2.48 (m, 2 H).

Using this reagent, the remainder of the synthesis is identical to that previously described in Example 4. 1H NMR (d₆-DMSO) δ ppm: 9.33 (s, 2 H), 9.01 (s, 2 H), 8.11 (d, 2 H, J=6 Hz), 7.75 (d, 1H, J=8.4 Hz), 7.68 (d, 1 H, J=8.0 Hz), 7.49 (d, 1 H, J=1.7 Hz), 7.24 (s, 1 H), 7.11 (d, 1 H, 1.5 Hz), 3.92 (s, 2 H), 3.92-3.86 (m, 1 H), 3.08 (dd, 1 H, J=17, 10 Hz), 2.61 (dd, 1 H, J=13, 4.9 Hz). MS LCMS Q⁺ 578.06 (calc.), 578.2 (obs).

### Example 13

### Synthesis of 2-[5-(5-carbamimidoyl-1H-benzimidazol-2-yl)-2′,6-dihydroxy-5′-(4-methylpiperazin-1-ylmethyl)biphenyl-3-yl]-succinic acid

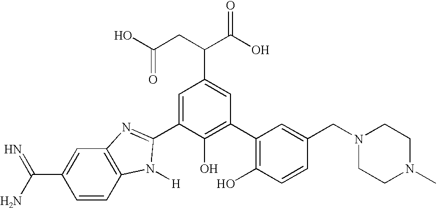

Step (a)

3-Bromo-4-hydroxybenzaldehyde (2.01 g, 10 mmol) was dissolved in 10 mL of THF which was subsequently charged with 110 mL of DCM. Diisopropylethylamine (1.94 g, 2.61 mL, 15.0 mmol) and MEM-chloride (1.87 g, 1.71 mL, 15.0 mmol) were then added and the solution was stirred overnight. The volatile solvent was evaporated and the residue was taken up in ethyl acetate, which was washed 3× with water and dried. Evaporation and pumping down the residue gave 3-bromo-4-(2-methoxyethoxymethoxy)benzaldehyde (2.94 g, 99%) which was used in the next step without further purification.

Step (b)

3-Bromo-4-(2-methoxyethoxymethoxy)benzaldehyde (0.355 g, 1.19 mmol) and 1-methylpiperazine (0.50 g, 0.55 mL, 5.0 mmol) are dissolved in 10 mL of MeOH at room temperature. This solution was charged with a solution consisting of sodium cyanoborohydride (94 mg, 1.5 mmol), zinc chloride (0.102 g, 0.75 mmol), in 5 mL of MeOH. This solution was stirred for 2 hours. 1N NaOH was added and the lot was extracted with chloroform which was washed several times with water. After drying and evaporation of the solvent, the yield of crude 1-[bromo-4-(2-methoxyethoxymethoxy)-benzyl]-4-methylpiperazine was 0.485 g (˜100%) which was used in the next step without further purification.

Step (c)

1-[3-Bromo-4-(2-methoxyethoxymethoxy)benzyl]-4-methylpiperazine (0.30 g, 0.80 mmol) was dissolved in a 0.1M toluene solution containing 0.80 mmol of dimethyl 2-[3-formyl-4-(2-methoxyethoxymethoxy)-5-(4,4,5,5-tetramethyl-[1,3,2]dioxaborolan-2-yl)phenyl]succinate (prepared as described in Example 4, Step (a)). This solution was charged with 2.0 mL of 1M sodium carbonate. After bubbling nitrogen through for one minute, tetrakis(triphenylphosphine)palladium(0) (0.116 g, 0.10 mmol) was added and the resultant mixture was refluxed for 6 hr. After workup of the reaction mixture (partitioned between 5% citric acid solution/ethyl acetate), the crude product was purified over 5 g of silica gel (1:1 ethyl acetate/hexane, ethyl acetate, 20% methanol/ethyl acetate) to give dimethyl 2-[5-formyl-6,2′-bis(2-methoxyethoxymethoxy)-5′-(4-methylpiperazin-1-yl methyl)biphenyl-3-yl]succinate (0.44 g (85%).

Step (d)

Dimethyl 2-[5-formyl-6,2′-bis-(2-methoxyethoxymethoxy)-5′-(4-methylpiperazin-1-ylmethyl)biphenyl-3-yl]succinate (0.40 g, 0.618 mmol) was dissolved in 15 mL of MeOH and was treated with 0.68 mmol (0.127 g) of 3,4-diaminobenzamidine mono hydrochloride and 0.74 mmol (80.2 mg) of benzoquinone. The reaction mixture was refluxed for 24 hours and then cooled. After evaporation of the solvent, it was redissolved in 5 mL of MeOH and then treated with 5 mL of 4M HCl in dioxane and stirred. After 1.5 hours, the solvents are evaporated and the crude residue was dissolved in 10 mL of a 1:1 solution of 3NHCl/acetonitrile and refluxed for 4 hours. The reaction mixture was cooled to ambient temperature and concentrated under reduced pressure. The residue was purified by reverse phase HPLC (gradient, acetonitrile/0.02 N aq HCl) to yield 131 mg (37%) of the title compound.

NMR (DMSO-d₆) δ ppm: 2.48 (s, 3H), 2.63 (d of d, J=6, 19 Hz, 1H), 3.14 (d of d, J=11, 19 Hz, 1H), 3.3-3.7 (m, 8H), 3.90 (d of d, J=6, 11 Hz, 1H), 4.31 (s, 2H), 7.00 (d, J=8 Hz, 1H), 7.36 (s, 1H), 7.46 (s, 1H), 7.47 (d, J=8 Hz, 1H), 7.74 (d, J=2 Hz, 1H), 7.83 (d, J=8 Hz, 1H), 8.13 (s, 1H), 8.19 (s, 1H), 9.16 (s, 2H), 9.42 (s, 2H).

### Example 14

### Synthesis of 2-[5-(5-carbamimidoyl-1H-benzimidazol-2-yl)-2′,6-dihydroxy-5′-(hydroxymethyl)biphenyl-3-yl]-succinic acid

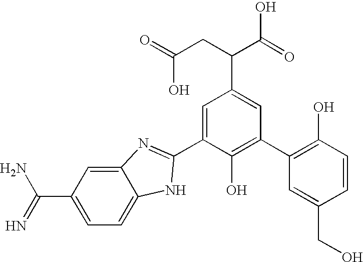

2-[5-(5-Carbamimidoyl-1H-benzoimidazol-2-yl)-5′-cyano-6,2′-dihydroxy-biphenyl-3-yl]-succinic acid (1.3 g, 2.7 mmol) was dissolved in a solution of water (100 mL) and 1N aqueous HCl (2 mL). Pearlman's catalyst (250 mg) was added and the mixture was stirred under one atmosphere of hydrogen for 6 hours. The solution was filtered through celite and purified by reverse phase HPLC (gradient, acetonitrile/0.02 N aqueous HCl_{conc}) to yield 2-[5′-aminomethyl-5-(5-carbamimidoyl-1H-benzoimidazol-2-yl)-6,2′-dihydroxy-biphenyl-3-yl]-succinic acid (284 mg, 22%) as the bis HCl salt and the title compound as a yellow powder (313 mg, 24%). ¹H NMR (d₆-DMSO) δ ppm: 9.32 (bs, 2H), 8.92 (bs, 2H), 8.15 (s, 1H), 8.03 (s, 1H), 7.83 (d, J=7.7 Hz, 1H), 7.71 (d, J=8.8 Hz, 1H), 7.25 (s, 1H), 7.11 (d, J=7.2 Hz, 2H), 6.85 (d, J=8.8 Hz, 1H), 4.40 (s, 2H), 3.91 (dd, J=10.4 Hz, 2.1 Hz, 1H), 3.11 (dd, J=17.1 Hz, 5.0 Hz, 1H), 2.65 (dd, J=17.1 Hz, 1.9 Hz, 1H). MS LCMS Q⁺ 491.47 (calc.), 491.1 (obs.), Q⁻ 489.47 (calc.), 489.2 (obs.)

### Example 15

### Synthesis of 2-[5-(5-carbamimidoyl-1H-benzimidazol-2-yl)-2′,6-dihydroxy-5′-fluorobiphenyl-3-yl]-succinic acid

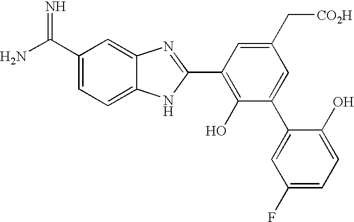

Step (a)

Commercially available 4-methoxyphenylacetic acid (16.6 g, 0.1 mol) was dissolved in acetic acid (120 mL ) and stirred vigorously at approximately 0° C. A solution of elemental bromine (16.0 g, 0.1 mol) in acetic acid (40 mL) was added dropwise over 45 minutes, ensuring that the mixture does not freeze. The reaction mixture was allowed to warm slowly to room temperature and stir overnight. Upon completion, most of the acetic acid was removed under reduced pressure and the residue partitioned between ethyl acetate and water. The organic layer was collected, washed with water, brine and 5% aqueous NaHSO3, filtered and the solvent was removed in vacuo to give with 3-bromo-4-methoxyphenylacetic acid (24.24 g, 98%) as a yellow powder.

Step (b)

A 250 mL round bottom flask was charged with 3-bromo-4-methoxyphenylacetic acid (14.0 g, 0.057 mol) and dry dichloromethane (100 mL). Nitrogen gas was bubbled through the reaction mixture for five minutes before a 1M solution of boron tribromide in dichloromethane (63 mL, 0.063 mol) was added very slowly via an addition funnel. The reaction mixture was allowed to run at room temperature and the desired white product gradually precipitates in the flask. After two hours the mixture was filtered and the collected crystals washed repeatedly with dichloromethane to give 3-bromo-4-hydroxyphenylacetic acid (12 g, 92%).

Step (c)

A solution of 3-bromo-4-hydroxyphenylacetic acid (12.0 g, 0.052 mol) in methanol was stirred at room temperature and ten drops of thionyl chloride were added. After two hours, the solvent was removed under reduced pressure and the residue was taken up in saturated aqueous sodium bicarbonate and extracted with diethyl ether (×3). The organic layers were collected, washed with water and brine, dried over MgSO₄ and concentrated in vacuo to give methyl-3-bromo-4-hydroxyphenylacetate as a golden oil (12.6 g, 99%).

Step (d)

Methyl-3-bromo-4-hydroxyphenylacetate was converted to methyl-3-formyl-4-hydroxy-5-bromophenylacetate following the formylation procedure in Reference 3, Step (b) above.

Step (e)

A 250 mL 24/40 round bottom flask was charged with 2-methoxymethylether-5-fluorophenylboronic acid (2.0 g, 10.0 mmol), methyl-3-formyl-4-hydroxy-5-bromophenylacetate (2.47 g, 9.0 mmol), dimethoxyethane (ethylene glycol dimethyl ether) (50 mL) and a magnetic stir bar. A 2 M aqueous solution of Na₂CO₃ was added (5.5 mL, 11 mmol) before the reaction vessel was purged with nitrogen for five minutes and the tetrakis(triphenylphosphine)palladium(0) (0.70 g, 0.60 mmol) added. The reaction was allowed to reflux for 3 hours before cooling to room temperature and neutralizing the solution with 5% aqueous citric acid to pH 4. The mixture was extracted with ethyl acetate (×4), the organic layers were collected and washed twice with water and brine before drying over MgSO4. The solution was filtered, concentrated under reduced pressure and purified using column chromatography (50 g silica, 85:15 Hex/EtOAc) to give methyl 2-[5-formyl-6-hydroxy-2′-(2-CH₃OCH₂O)-5′fluorobiphenyl-3-yl]acetate (2.2 g, 70%) as a yellow oil which crystallized overnight.

Step (f)

A mixture of give methyl 2-[5-formyl-6-hydroxy-2′-(2-CH₃OCH₂O)-5′fluorobiphenyl-3-yl]acetate (400 mg, 1.1 mmol), 3,4-diaminobenzamidine mono hydrochloride (235 mg, 1.2 mmol) and benzoquinone (125 mg, 1.15 mmol) in ethanol (50 mL) was heated at reflux for two hours. The solvent was removed under reduced pressure and the residue taken up in 2 mL HClconc, 4 mL water and 4 mL acetonitrile. After stirring at room temperature for one hour, the mixture was purified by reverse phase HPLC (gradient, acetonitrile/0.02 N aqueous HCl) to give (201 mg, 40%) of the title compound. ¹H NMR (d₆-DMSO) δ ppm: 9.35 (bs, 2H), 9.00 (bs, 2H), 8.15 (s, 1H), 8.16 (s, 1H), 8.02 (d, J=1.8 Hz, 1H), 7.83 (d, J=8.4 Hz, 1H), 7.72 (dd, J=8.4 Hz, 1.8 Hz, 1H), 7.30 (d, J=2.2, 1H), 7.00 (m, 2H), 6.90 (dd, J=10.5 Hz, 5.8 Hz, 1H), 3.71 (s, 2H). MS LCMS Q⁺ 421.39 (calc.), 420.9 (obs.), Q⁻ 419.39 (calc.), 419.2 (obs.)

### Example 16

### Synthesis of 2-{5-[5-carbamimidoyl-1H-pyrrolo[3,2-b]pyridin-2-yl)-5′-fluoro-6,2-dihydroxy]biphen-3-yl}acetic acid

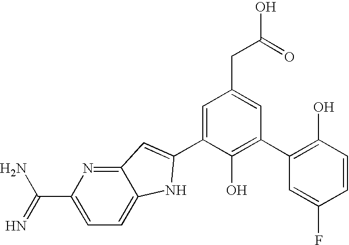

Step (a)

To a solution of methyl 2-{5-[2-(5-cyano-2-tert-butoxycarbonylamino)phenyl-ethynyl]-2′,6-bis-(methoxyethoxymethoxy)-5′-fluorobiphenyl}acetate (0.64 gm, 0.9 mmol), prepared from 6-bromo-5-(tert-butoxycarbonylamino)-3-chloro-2-cyano-pyridine and methyl-2-[5-ethynyl-2′,6-bis-methoxyethoxymethoxy)-5′-fluorobiphenyl]acetate as described in 9 above, in MeOH (75 mL) was added aqueous NaOH (10%, 2 mL) and the reaction mixture stirred at 50° C. for 1 hr. Citric acid (5% aqueous) and ethyl acetate were added and the organic layer was washed with water and brine and concentrated to afford (0.5 gm, 93%) of 2-{5-[(6-chloro-5-cyano-1H-pyrrolo[3,2-b]pyridin-2-yl)-5′-fluoro-2′,6-bis-(methoxyethoxymethoxy)]biphen-3-yl}acetic acid as an oil.

Step (b)

A solution of 2-{5-[(6-chloro-5-cyano-1H-pyrrolo[3,2-b]pyridin-2-yl)-5′-fluoro-2′,6-bis-(methoxyethoxymethoxy)]biphen-3-yl}acetic acid (0.44 gm, 0.77 mmol) in ethanol (15 mL) was treated with hydroxylamine (50% aqueous, 3 mL) and the mixture refluxed for 2 hrs. The reaction mixture was concentrated to dryness and dried under high vacuum overnight to afford 2-{5-[(6-chloro-5-(hydroxycarbamimidoyl)-1H-pyrrolo[3,2-b]pyridin-2-yl)-5′-fluoro-2′,6-bis-(methoxyethoxymethoxy)]biphen-3-yl}acetic acid (0.44 gm, 95%).

Step (c)

2-{5-[(6-Chloro-5-(hydroxycarbamimidoyl)-1H-pyrrolo[3,2-b]pyridin-2-yl)-5′-fluoro-6,2′-dihydroxy]biphen-3-yl}acetic acid (0.43 gm, 0.73 mmol) was dissolved in 10 mL acetic acid and treated with acetic anhydride (2 mL) and the mixture stirred for 1 hr. The rection mixture was concentrated and dried on a high vacuum, redissolved in minimum acetonitrile and the desired product was crashed out of water to afford 2-{5-[(6-chloro-5-(acetoxycarbamimidoyl)-1H-pyrrolo[3,2-b]pyridin-2-yl)-5′-fluoro-2′,6-bis-(methoxyethoxymethoxy)]biphen-3-yl}acetic acid as a brown solid (0.43 gm, 92%).

Step (d)

2-{5-[(6-Chloro-5-(acetoxycarbamimidoyl)-1H-pyrrolo[3,2-b]pyridin-2-yl)-5′-fluoro-2′,6-bis-(methoxyethoxymethoxy)]biphen-3-yl}acetic acid (0.11 gm, 0.17 mmol) was dissolved in methanol and Pearlman's catalyst (10% on activated carbon) was suspended in the solution. The reaction mixture was subjected to hydrogen under 1 atm for 2 hrs. The reaction mixture was filtered, concentrated and dried under high vacuum to afford 2-{5-[(5-(carbamimidoyl)-1H-pyrrolo[3,2-b]pyridin-2-yl)-5′-fluoro-2′,6-bis-(methoxyethoxymethoxy)]biphen-3-yl}acetic acid (0.097 gm, 99%).

Step (e)

2-{5-[(5-(Carbamimidoyl)-1H-pyrrolo[3,2-b]pyridin-2-yl)-5′-fluoro-2′,6-bis-(methoxyethoxymethoxy)]biphen-3-yl}acetic acid was redissolved in acetic acid (10 mL), treated with HCl (4 M in dioxane, 2 mL) and the mixture stirred for 1 hr. The resulting product was concentrated and purified by reverse phase HPLC to afford 18 mg (24%) of the desired product as an orange solid. LCMS calcd 420.1; found 421.2 (M+1). 1H NMR (DMSO-d6) δ ppm: 12.04 (s, 1H), 10.08 (s, 1H), 9.38 (s, 2H), 8.97 (s, 2H), 8.83 (s, 1H), 8.02 (d, 1H, J=8.5 Hz), 7.98 (d, 1H, J=8.5 Hz), 7.70 (s, 1H), 7.21 (s, 1H), 7.15 (s, 1H), 7.10-6.96 (m, 3H), 3.63 (s, 2H).

### Biological Examples

### Example 1

### In Vitro Factor VIIa Inhibitor Assay

Mixtures of human Factor VIIa (typically supplied at 7 nM) and test compound (present at varying concentrations) in assay medium (comprising: NaCl, 150 mM (pH 7.4); CaCl₂, 5 mM; Tween-20, 0.05% ; Dade Innovin tissue factor [Dade Behring, Newark, Del., USA]; EDTA, 1.5 mM; and dimethylsulfoxide, 10%) were incubated for 30 minutes at room temperature. Next, reactions were initiated with the addition of substrate [500 μM of CH₃SO₂-D-Cha-But-Arg-pNA (from Centerchem, Norwalk, Conn., USA)]. Hydrolysis of the chromogenic substrate was followed spectrophotometrically at 405 nm for five minutes. Initial velocity measurements calculated from the progress curves by a kinetic analysis program (Batch Ki; BioKin, Ltd., Pullman, Wash.) were used to determine apparent inhibition constants (apparent K₁'s).

Compounds of the invention tested by the above-described assay exhibited inhibition of Factor VIIa.

### Example 2

### In Vitro Factor Xa Inhibitor Assay

Mixtures of human Factor Xa (typically supplied at 3 nM) (from Haematologic Technologies, Essex Junction, Vt., USA) and test compound (varying concentrations) in assay medium (comprising: Tris, 50 mM (pH 7.4); NaCl, 150 mM; CaCl₂, 5 mM; Tween-20, 0.05%; EDTA, 1 mM; and dimethylsulfoxide, 10%) were incubated for 30 minutes at room temperature. Next, reactions were initiated with the addition of substrate [500 μM of CH₃CO₂-D-Cha-Gly-Arg-pNA (from Centerchem, Norwalk, Conn., USA]. Hydrolysis of the chromogenic substrate was followed spectrophotometrically at (405 nm) for five minutes. Apparent inhibition constants (apparent K₁'s) were calculated from the enzyme progress curves using standard mathematical models.

Compounds of the invention tested by the above-described assay exhibited inhibition of Factor Xa.

### Example 3

### Pharmacokinetic Assay

Rats with pre-implanted jugular vein catheters, which were filled with heparin/saline/PVP lock prior to shipment, were bought from Charles River. Three rats were selected for each study, weighed, and injected with test compound by tail vein injection. Any residual test compound was retained and stored at −70° C. for later analysis.

Blood samples (0.25 mL each) were collected from the indwelling catheters at specified times over 120 hours. The catheters were flushed with physiological saline immediately after each collection and filled with heparinized saline after each 8, 24 and 48 hour collection. In the event that a catheter failed, blood samples were collected via the retro-orbital sinus under isoflurane anesthesia at the appropriate time.

Blood samples were placed in 0.5 mL Microtainer® tubes (lithium heparin), shaken gently and stored on wet ice. The samples were centrifuged for 10 minutes at 2400 rpm in a refrigerated centrifuged. Plasma samples (0.1 mL) from each tube were transferred to 0.5 mL Unison polypropylene vials (Sun-500210) and stored below −70° C. for later analysis by LC/MS-MS.

### Example 4

### In Vitro Clotting Assays . . . aPTT and PT

Coagulation assays, activated partial thromboplastin time (aPTT) and prothrombin time (PT) were carried out based on the procedure described in Hougie, C. Hematology (Williams, W. J., Beutler, B., Erslev, A. J., and Lichtman, M. A., Eds.), pp. 1766-1770 (1990), McGraw-Hill, New York.

Briefly, the assays were performed using normal human citrated plasma and were performed at 37° C. on a coagulometer (Electra 800) in accordance with the manufacturer's instructions (Medical Laboratory Automation—Pleasantville, N.Y.). The instrument was calibrated with plasma immediately prior to collecting clotting times for samples with inhibitors. The aPTT and PT doubling concentrations were calculated by fitting inhibitor dose response curves to a modified version of the Hill equation.

### Pharmaceutical Composition Examples

The following are representative pharmaceutical formulations containing a compound of Formula I.

Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | mL |
| colorings | 0.5 | mg |
| distilled water | q.s. to 100 | mL |

Injectable Formulation

The following ingredients are mixed to form an injectable formulation.

| Ingredient | Amount |
|---|---|
| compound of this invention | 1.2 g |
| sodium acetate buffer solution, | 0.4 M 2.0 mL |
| HCl (1 N) or NaOH (1 N) | q.s. to suitable pH |
| water (distilled, sterile) | q.s. to 20 mL |

All of the above ingredients, except water, are combined and heated to 60-70° C. with stirring. A sufficient quantity of water at 60° C. is then added with vigorous stirring to emulsify the ingredients, and water then added q.s. to 100 g.

Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| | |
|---|---|
| compound of the invention | 500 mg |
| Witepsol ® H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

We claim:

1. A compound of Formula I: 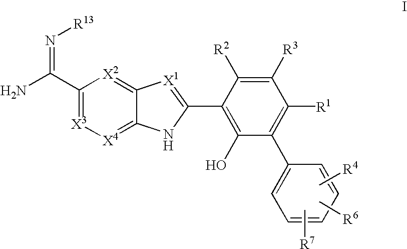
wherein:
X¹, X², X³, and X⁴ are independently —N— or —CR⁵— wherein R⁵ is hydrogen, alkyl, or halo with the proviso that not more than three of X¹, X², X³ and X⁴ are —N—;
R¹ and R² independently are hydrogen, alkyl, or halo;
R³ is —COOR⁹, -(alkylene)-COOR⁹, —CR⁸(COOR¹¹)alkylene-COOR⁹, or a group of formula (a): 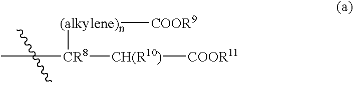
where:
n is 0 or 1;
R⁸ is hydrogen, alkyl, or hydroxy; and
R¹⁰ is hydrogen or alkyl; or
R⁸ and R¹⁰ together form a covalent bond;
R⁹ and R¹¹ are independently hydrogen, alkyl, haloalkyl, aryl, or aralkyl;
R⁴ is hydrogen, alkyl, alkylthio, halo, hydroxy, hydroxyalkyl, alkoxy, aminosulfonyl, alkylaminosulfonyl, dialkylaminosulfonyl, or nitro;
R⁶ is hydrogen, alkyl, or halo;
R⁷ is hydrogen, alkyl, cycloalkyl, alkylthio, halo, hydroxy, nitro, cyano, alkoxy, haloalkoxy, carboxy, alkoxycarbonyl, acylamino, alkylsulfonyl, arylsulfonyl, heteroarylsulfonyl, carbamimidoyl, hydroxycarbamimidoyl, alkoxycarbamimidoyl, alkylsulfonylamino, alkoxysulfonylamino, alkylsulfonylaminoalkyl, alkoxysulfonylaminoalkyl, heterocycloalkylalkylaminocarbonyl, hydroxyalkoxyalkylaminocarbonyl, haloalkyl, cyanoalkyl, alkoxyalkyl, hydroxyalkyl, carboxyalkyl, alkoxycarbonylalkyl, heterocycloalkylcarbonyl, heterocycloalkylcarbonylalkyl, heterocycloalkyl, heterocycloalkylalkyl, oxoheterocycloalkylalkyl, aminosulfonylalkyl, heteroaryl, heteroaralkyl, ureido, alkylureido, dialkylureido, ureidoalkyl, alkylureidoalkyl, dialkylureidoalkyl, thioureido, thioureidoalkyl, —COR¹² (where R¹² is alkyl or haloalkyl), -(alkylene)-COR¹² (where R¹² is alkyl or haloalkyl), aminocarbonyl, aminocarbonylalkyl, —CONR¹⁴R¹⁵ (where R¹⁴ is hydrogen or alkyl and R¹⁵ is alkyl, aryl, aralkyl, heteroaryl, or heteroaralkyl), -(alkylene)-CONR¹⁶R¹⁷ (where R¹⁶ is hydrogen or alkyl and R¹⁷ is alkyl, aryl, aralkyl, heteroaryl, or heteroaralkyl), amino, alkylamino, dialkylamino, —NR¹⁸R¹⁹ (where R¹⁸ is hydrogen or alkyl and R¹⁹ is aryl, aralkyl, heteroaryl, or heteroaralkyl), aminoalkyl, -(alkylene)-NR²⁰R²¹ (where R²⁰ is hydrogen or alkyl and R²¹ is alkyl, aryl, aralkyl, heteroaryl, or heteroaralkyl), aminosulfonyl, —SO₂NR²²R²³ (where R²² is hydrogen or alkyl and R²³ is alkyl, aryl, aralkyl, heteroaryl, or heteroaralkyl, or R²² and R²³ together with the nitrogen atom to which they are attached from heterocycloamino), -(alkylene)-SO₂NR²⁴R²⁵ (where R²⁴ is hydrogen or alkyl and R²⁵ is alkyl, aryl, aralkyl, heteroaryl, or heteroaralkyl or R²⁴ and R²⁵ together with the nitrogen atom to which they are attached from heterocycloamino), aminosulfonylamino, —NR²⁶SO₂NR²⁷R²⁸ (where R²⁶ and R²⁷ are independently hydrogen or alkyl, and R²⁸ is alkyl, aryl, aralkyl, heteroaryl, or heteroaralkyl or R²⁷ and R²⁸ together with the nitrogen atom to which they are attached from heterocycloamino), -(alkylene)-NR²⁹SO₂NR³⁰R³¹ (where R²⁹ and R³⁰ are independently hydrogen or alkyl, and R³¹ is hydrogen, alkyl, aryl, aralkyl, heteroaryl, or heteroaralkyl or R³⁰ and R³¹ together with the nitrogen atom to which they are attached from heterocycloamino), —CONH-(alkylene)-NR³²R³³ where R³² is hydrogen or alkyl and R³³ is alkyl), or -(alkenylene)-R³⁴ (where R³⁴ is alkyl, alkoxy, carboxy, alkoxycarbonyl, amino, alkylamino, dialkylamino, acylamino, aminosulfonylamino, alkylaminosulfonylamino, alkylsulfonyl, aryl, heteroaryl, heterocycloalkyl, heterocycloalkylcarbonyl, aminocarbonyl, aminosulfonyl, —COR¹², —CONR¹⁴R¹⁵, —NR¹⁸R¹⁹, —SO₂NR²²R²³, or—NR²⁶SO₂NR²⁷R²⁸ where R¹², R¹⁴, R¹⁵, R¹⁸ , R¹⁹, R²², R²³, R²⁶, R²⁷, and R²⁸ are as defined above); and
R¹³ is hydrogen, hydroxy, (C₁₋₁₀)alkoxy, —C(O)R³⁵ where R³⁵ is alkyl, aryl, haloalkyl, or cyanoalkyl, or —C(O)OR³⁶ where R³⁶ is alkyl, hydroxyalkyl, acyl, or haloalkyl; and individual isomers, mixture of isomers, or a pharmaceutically acceptable salt thereof, provided that when R⁷ is hydrogen, alkyl, halo, nitro, alkoxy, haloalkyl, carboxy, alkoxycarbonyl, amino, alkylamino, dialkylamino, —NR¹⁸R¹⁹ (where R¹⁸ is hydrogen or alkyl and R¹⁹ is aryl or aralkyl), pyrrolidinylcarbonyl, —SO₂NR²²R²³ (where R²² and R²³ are alkyl), carbamimidoyl, alkylsulfonylamino, alkylthio, ureido or —NHC(S)NH₂, and R³ is —COOR⁹, -(alkylene)-COOR⁹, —CR⁸(COOR¹¹)alkylene-COOR⁹, or a group of formula (a) where n is 0 or 1; R⁸ and R¹⁰ are independently hydrogen or alkyl, and R¹³ is hydrogen; then R⁴ is hydroxy or hydroxyalkyl.

2. The compound of claim 1 wherein X is —N— and X², X³, and X⁴ are —CR⁵— where R⁵ is hydrogen.

3. The compound of claim 1 wherein X¹ is —N—; X² and X⁴ are —CR⁵— where R⁵ is hydrogen and X³ is —CR⁵— where R⁵ is halo.

4. The compound of claim 1 wherein X¹ is —CH— and X², X³, and X⁴ are —CR⁵— where R⁵ is hydrogen.

5. The compound of claim 1 wherein X¹ is —CH—; x² and X⁴ are —CR⁵— where R⁵ is hydrogen and X³ is —CR⁵— where R⁵ is halo.

7. The compound of claim 2 where R¹, R², and R¹³ are hydrogen; R³ is a group of formula (a) where n is 0, R⁸ and R¹⁰ are hydrogen and one of R⁹ and R¹¹ is hydrogen and the other of R⁹ and R¹¹ is ethyl.

8. The compound of claim 2 where R¹, R², and R¹³ are hydrogen; R³ is a group of formula (a) where n is 0, R⁸, R⁹, R¹⁰ and R¹¹ are hydrogen.

9. The compound of claim 4 where R¹, R², and R¹³ are hydrogen; R³ is a group of formula (a) where n is 0, R⁸ and R¹⁰ are hydrogen and one of R⁹ and R¹¹ is hydrogen and the other of R⁹ and R¹¹ is ethyl.

10. The compound of claim 4 where R¹, R², and R³ are hydrogen; R³ is a group of formula (a) where n is 0, R⁸, R⁹, R¹⁰ and R¹¹ are hydrogen.

11. The compound of claim 8 wherein R⁴ is hydroxy or hydroxymethyl and is located at the 2′-position of the biphenyl ring and R⁶ and R⁷ are hydrogen.

12. The compound of claim 8 wherein R⁴ is hydroxy and is located at the 2′-position of the biphenyl ring, R⁶ is hydrogen, and R⁷ is located at the 5′-position of the biphenyl ring.

13. The compound of claim 12 wherein R⁷ is alkyl, halo, hydroxy, hydroxyalkyl, carboxy, alkoxy, cyano, nitro, amino, aminocarbonyl, alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

14. The compound of claim 12 wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, acetyl, or aminosulfonyl.

15. The compound of claim 8 wherein R⁴ is hydroxymethyl and is located at the 2′-position of the biphenyl ring, R⁶ is hydrogen, and R⁷ is located at the 5′-position of the biphenyl ring.

16. The compound of claim 15 wherein R⁷ is alkyl, halo, hydroxy, hydroxyalkyl, carboxy, alkoxy, cyano, nitro, amino, aminocarbonyl, alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

17. The compound of claim 15 wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, acetyl, or aminosulfonyl.

18. The compound of claim 8 wherein R⁴ is aminosulfonyl and is located at the 2′-position of the biphenyl ring, R⁶ is hydrogen, and R⁷ is located at the 5′-position of the biphenyl ring.

19. The compound of claim 18 wherein R⁷ is alkyl, halo, hydroxy, hydroxyalkyl, carboxy, alkoxy, cyano, nitro, amino, aminocarbonyl, alkylsulfonylamino, aminoalkyl, aminosulfonyl, ureido, ureidoalkyl, alkylureidoalkyl, cyanoalkyl, carboxyalkyl, aminocarbonylalkyl, heteroaryl, heterocycloalkylcarbonyl, heterocycloalkylalkyl, —NHSO₂NR²⁷R²⁸ where R²⁷ and R²⁸ are independently hydrogen or alkyl, or —COR¹² where R¹² is alkyl.

20. The compound of claim 18 wherein R⁷ is methyl, isopropyl, chloro, fluoro, hydroxy, hydroxymethyl, 2-hydroxyethyl, carboxy, methoxy, cyano, nitro, aminocarbonyl, methylsulfonylamino, aminomethyl, ureidomethyl, —CH₂NHCONHCH₃, imidazol-2-yl, amino, ureido, cyanomethyl, 2-cyanoethyl, carboxymethyl, 2-carboxyethyl, aminocarbonylmethyl, dimethylaminosulfonylamino, or acetyl.

21. The compound of claim 8 wherein R⁴ and R⁵ are hydrogen and R⁷ is located at the 3′-position of the biphenyl ring.

22. The compound of claim 21 wherein R⁷ is aminosulfonyl, haloalkoxy, hydroxy, hydroxyalkyl, aminocarbonyl, ureidoalkyl, cyanoalkyl, alkoxyalkyl, carboxyalkyl, aminocarbonylalkyl, heterocycloalkylalkyl, —COR¹² (where R¹² is alkyl) or cyano.

23. The compound of claim 1 wherein the moiety: 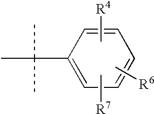
is 2′-acetylphenyl, 3′-acetylphenyl, 3′-hydroxyphenyl, 2′-hydroxyphenyl, 3′-aminocarbonylphenyl, 3′-cyanophenyl, 5′-fluoro-2′-hydroxyphenyl, 5′-chloro-2′-hydroxy-phenyl, 2′-hydroxy-methylphenyl, 5′-carboxy-2′-hydroxyphenyl, 2′,5′-dihydroxyphenyl, 5′-cyano-2′-methoxyphenyl, 5′-aminocarbonyl-2′-methoxyphenyl, 2′,6′-dihydroxyphenyl, 3′-bromo-2′,6′-dihydroxyphenyl, 2′-hydroxy-5′-nitrophenyl, 2′-cyano-phenyl, 3′-hydroxymethylphenyl, 3′-(2-hydroxyethylphenyl), 5′-cyano-2′-hydroxyphenyl, 5′-aminocarbonyl-2′-hydroxyphenyl, 5′-aminomethyl-2′-hydroxyphenyl, 2′-hydroxy-5′-ureidomethylphenyl, 2′-hydroxy-5′-imidazol-2-ylphenyl, 5′-amino-2′-hydroxyphenyl, 2′-hydroxy-5′-ureidophenyl, 2′-hydroxy-5′-(2-morpholin-4-ylethyl)aminocarbonylphenyl, 3′-bromo-2′-hydroxy-5′-cyanomethylphenyl, 5′-(2-cyanoethyl)-2′-hydroxyphenyl, 3′-bromo-5′-carboxymethyl-2′-hydroxyphenyl, 5′-(2-carboxyethyl)-2′-hydroxyphenyl, 5′-aminocarbonylmethyl-2′-hydroxyphenyl, 3′,5′-dichloro-2′-hydroxyphenyl, 2′-hydroxy-5′-[2-(2-hydroxyethoxy)-ethylaminocarbonyl]phenyl, 5′-dimethylaminosulfonylamino-2′-hydroxy-phenyl, 3′-bromo-5′-chloro-2′-hydroxyphenyl, 2′-hydroxy-5′-(4-methylpiperazin-1-ylcarbonyl)phenyl, 2′-hydroxy-5′-(4-methylpiperazin-1-ylmethyl)phenyl, 5′-amidino-2′-hydroxyphenyl, 5′-(2-dimethylaminoethylaminocarbonyl)-2′-hydroxyphenyl, 3′-aminosulfonylphenyl, 2′-hydroxy-5′-aminosulfonylphenyl, 2′-hydroxy-5′-hydroxymethyl-phenyl, 2′-hydroxy-5′-(2-hydroxyethyl)phenyl, 2′-hydroxy-5′-dimethylaminosulfonyl-aminophenyl, 5′-aminocarbonyl-2′-hydroxy-phenyl, or 2′-hydroxy-5′-(CH₃NHCONHCH₂)phenyl.

24. The compound of claim 1 wherein the moiety: 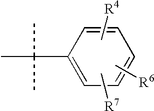
is 2′,6′-dihydroxyphenyl, 5′-fluoro-2′-hydroxyphenyl, 5′-aminocarbonyl-2′-hydroxyphenyl, 3′-aminosulfonylphenyl, 3′-ureidomethylphenyl, 2′-hydroxy-5′-hydroxymethylphenyl, or 2′-hydroxy-5′-ureidomethylphenyl.

25. A compound selected from the group consisting of:
2-[5-(5-carbamimidoyl-1H-benzoimidazol-2-yl)-3′-(1,1-difluoro-methoxy)-6-hydroxy-biphenyl-3-yl]-succinic acid;
2-[3′-acetyl-5-(5-carbamimidoyl-1H-benzoimidazol-2-yl)-6-hydroxy-biphenyl-3-yl]-succinic acid;
2-[5-(5-carbamimidoyl-1H-benzoimidazol-2-yl)-6,3′-dihydroxy-biphenyl-3-yl]-succinic acid;
2-[5-(5-carbamimidoyl-1H-benzoimidazol-2-yl)-6,2′-dihydroxy-biphenyl-3-yl]-succinic acid;
2-[5-(5-carbamimidoyl-1H-benzoimidazol-2-yl)-3′-aminocarbonyl-6-hydroxy-biphenyl-3-yl]-succinic acid;
2-[5-(5-carbamimidoyl-1H-benzoimidazol-2-yl)-3′-cyano-6-hydroxy-biphenyl-3-yl]-succinic acid;
2-[5-(5-carbamimidoyl-1H-benzoimidazol-2-yl)-5′-fluoro-6,2′-dihydroxy-biphenyl-3-yl]-succinic acid;
2-[5-(5-carbamimidoyl-1H-benzoimidazol-2-yl)-5′-chloro-6,2′-dihydroxy-biphenyl-3-yl]-succinic acid;
2-[5-(5-carbamimidoyl-1H-benzoimidazol-2-yl)-6-hydroxy-2′-hydroxymethyl-biphenyl-3-yl]-succinic acid;
2-[5-(5-carbamimidoyl-1H-indol-2-yl)-6,2′-dihydroxy-biphenyl-3-yl]-succinic acid;
2-[5-(5-carbamimidoyl-1H-benzoimidazol-2-yl)-5′-carboxy-6,2′-dihydroxy-biphenyl-3-yl]-succinic acid;
2-[5-(5-carbamimidoyl-1H-benzoimidazol-2-yl)-6,2′,5′-trihydroxy-biphenyl-3-yl]-succinic acid;
2-[5-(5-carbamimidoyl-1H-benzoimidazol-2-yl)-5′-cyano-6-hydroxy-2′-methoxy-biphenyl-3-yl]-succinic acid;
2-[5-(5-carbamimidoyl-1H-benzoimidazol-2-yl)-5′-aminocarbonyl-6-hydroxy-2′-methoxy-biphenyl-3-yl]-succinic acid;
2-[5-(5-carbamimidoyl-1H-benzoimidazol-2-yl)-6,2′,6′-trihydroxy-biphenyl-3-yl]-succinic acid;
2-[5-(5-carbamimidoyl-1H-benzoimidazol-2-yl)-6,2′-dihydroxy-5′-nitro-biphenyl-3-yl]-succinic acid;
2-[5-(5-carbamimidoyl-1H-benzoimidazol-2-yl)-2′-cyano-6-hydroxy-biphenyl-3-yl]-succinic acid;
2-[5-(6-carbamimidoyl-1H-benzoimidazol-2-yl)-6-hydroxy-3′-hydroxymethyl-biphenyl-3-yl]-succinic acid;
2-[5-(5-carbamimidoyl-1H-benzoimidazol-2-yl)-5′-cyano-6,2′-dihydroxy-biphenyl-3-yl]-succinic acid;
2-[5-(5-carbamimidoyl-1H-benzoimidazol-2-yl)-5′-aminocarbonyl-6,2′-dihydroxy-biphenyl-3-yl]-succinic acid;
2-[3′-bromo-5-(5-carbamimidoyl-1H-benzoimidazol-2-yl)-6,2′,6′-trihydroxy-biphenyl-3-yl]-succinic acid;
2-[5-(5-carbamimidoyl-1H-benzoimidazol-2-yl)-6,2′-dihydroxy-5′-methylsulfonylamino-biphenyl-3-yl]-succinic acid;
2-[5-(5-carbamimidoyl-1H-benzoimidazol-2-yl)-6,2′-dihydroxy-5′-isopropyl-biphenyl-3-yl]-succinic acid;
2-[5′-aminomethyl-5-(5-carbamimidoyl-1H-benzoimidazol-2-yl)-6,2′-dihydroxy-biphenyl-3-yl]-succinic acid;
2-[5-(5-carbamimidoyl-1H-benzoimidazol-2-yl)-6,2′-dihydroxy-5′-ureidomethyl-biphenyl-3-yl]-succinic acid;
2-[5-(5-carbamimidoyl-1H-benzoimidazol-2-yl)-6,2′-dihydroxy-5′-imidazol-2-yl-biphenyl-3-yl]-succinic acid;
2-[5′-amino-5-(5-carbamimidoyl-1H-benzoimidazol-2-yl)-6,2′-dihydroxy-biphenyl-3-yl]-succinic acid;
2-[5-(5-carbamimidoyl-1H-benzoimidazol-2-yl)-6,2′-dihydroxy-5′-ureido-biphenyl-3-yl]-succinic acid;
2-[5-(5-carbamimidoyl-1H-benzoimidazol-2-yl)-6,2′-dihydroxy-5′-(2-morpholin-4-ylethylaminocarbonyl-biphenyl-3-yl]-succinic acid;
2-[3′-bromo-5-(5-carbamimidoyl-1H-benzoimidazol-2-yl)-6,2′-dihydroxy-5′-cyanomethylbiphenyl-3-yl]-succinic acid;
2-[5-(5-carbamimidoyl-1H-benzoimidazol-2-yl)-5′-(2-cyanoethyl)-6,2′-dihydroxybiphenyl-3-yl]-succinic acid;
2-[3′bromo-5-(5-carbamimidoyl-1H-benzoimidazol-2-yl)-5′-carboxymethyl-6,2′-dihydroxy-biphenyl-3-yl]-succinic acid;
2-[5-(5-carbamimidoyl-1H-benzoimidazol-2-yl)-5′-(2-carboxyethyl)-6,2′-dihydroxy-biphenyl-3-yl]-succinic acid;
2-[2′-acetyl-5-(5-carbamimidoyl-1H-benzoimidazol-2-yl)-6-hydroxy-biphenyl-3-yl]-succinic acid;
2-[3′-bromo-5-(5-carbamimidoyl-1H-benzoimidazol-2-yl)-6,2′-dihydroxy-5′-aminocarbonylmethyl-biphenyl-3-yl]-succinic acid;
2-[5-(5-carbamimidoyl-1H-indol-2-yl)-5′-aminocarbonyl-6,2′-dihydroxy-biphenyl-3-yl]-succinic acid;
2-[5-(5-carbamimidoyl-1H-benzoimidazol-2-yl)-3′,5′-dichloro-6,2′-dihydroxy-biphenyl-3-yl]-succinic acid;
2-[5-(5-carbamimidoyl-1H-benzoimidazol-2-yl)-6,2′-dihydroxy-5′-[2-(2-hydroxyethoxy)ethylaminocarbonyl]-biphenyl-3-yl]-succinic acid;
2-[5-(5-carbamimidoyl-1H-benzoimidazol-2-yl)-4′,6′-dichloro-6,2′-dihydroxy-biphenyl-3-yl]-succinic acid;
2-[5-(5-carbamimidoyl-1H-benzoimidazol-2-yl)-6,2′-dihydroxy-5′-dimethylaminosulfonylamino-biphenyl-3-yl]-succinic acid;
2-[3′-bromo-5-(5-carbamimidoyl-1H-benzoimidazol-2-yl)-5′-chloro-6,2′-dihydroxy-biphenyl-3-yl]-succinic acid;
2-[5-(5-carbamimidoyl-1H-benzoimidazol-2-yl)-6,2′-dihydroxy-5′-(4-methyl-piperazin-1-ylcarbonyl)-biphenyl-3-yl]-succinic acid;
2-[5-(5-carbamimidoyl-1H-benzoimidazol-2-yl)-6,2′-dihydroxy-5′-(4-methyl-piperazin-1-ylmethyl)-biphenyl-3-yl]-succinic acid;
2-[5-(5-carbamimidoyl-1H-benzoimidazol-2-yl)-5′-carbamimidoyl-6,2′-dihydroxy-biphenyl-3-yl]-succinic acid;
2-[5-(5-carbamimidoyl-1H-benzoimidazol-2-yl)-5′-(2-dimethylaminoethylaminocarbonyl)-6,2′-dihydroxy-biphenyl-3-yl]-succinic acid;
2-[5-(5-carbamimidoyl-1H-benzoimidazol-2-yl)-6,2′-dihydroxy-5′-hydroxymethyl-biphenyl-3-yl]-succinic acid;
2-[5-(5-carbamimidoyl-1H-benzoimidazol-2-yl)-6,2′-dihydroxy-5′-methylureidomethylbiphenyl-3-yl]-succinic acid;
2-[3′-aminosulfonyl-5-(5-carbamimidoyl-1H-benzoimidazol-2-yl)-6-hydroxy-biphenyl-3-yl]-succinic acid;
2-[5-(5-carbamimidoyl-1H-benzoimidazol-2-yl)-5′-dimethylaminosulfonyl-amino-6,2′-dihydroxy-biphenyl-3-yl]-succinic acid;
2-[5-(5-carbamimidoyl-6-fluoro-1H-benzoimidazol-2-yl)-6,2′-dihydroxy-biphenyl-3-yl]-succinic acid;
2-[5-(5-carbamimidoyl-6-chloro-1H-indol-2-yl)-6,2′-dihydroxy-biphenyl-3-yl]-succinic acid;
diethyl 2-[5-(5-carbamimidoyl-1H-benzoimidazol-2-yl)-5′-fluoro-6,2′-dihydroxy-biphenyl-3-yl]-succinate;
2-[5-(5-carbamimidoyl-5-fluoro-1H-benzoimidazol-2-yl)-5′-fluoro-6,2′-dihydroxy-biphenyl-3-yl]-succinic acid;
2-[5-(5-carbamimidoyl-1H-benzoimidazol-2-yl)-5′-fluoro-6,2′-dihydroxy-biphenyl-3-yl]-2-methylsuccinic acid;
2-[5-(5-carbamimidoyl-1H-benzoimidazol-2-yl)-5′-fluoro-6,2′-dihydroxy-biphenyl-3-yl]-succinic acid 1-ethyl ester;
2-[5-(5-carbamimidoyl-1H-benzoimidazol-2-yl)-5′-fluoro-6,2′-dihydroxy-biphenyl-3-yl]-succinic acid 4-ethyl ester;
(Z)-2-[5-(5-carbamimidoyl-1H-benzoimidazol-2-yl)-5′-fluoro-6-hydroxy-2′methoxy-biphenyl-3-yl]-but-2-enedioic acid;
(Z)-2-[5-(5-carbamimidoyl-1H-benzoimidazol-2-yl)-5′-fluoro-6,2′-dihydroxy-biphenyl-3-yl]-but-2-enedioic acid;
(E)-2-[5-(5-carbamimidoyl-1H-benzoimidazol-2-yl)-5′-fluoro-6,2′-dihydroxy-biphenyl-3-yl]-but-2-enedioic acid;
3-[5-(5-carbamimidoyl-1H-benzoimidazol-2-yl)-5′-fluoro-6,2′-dihydroxy-biphenyl-3-yl]-propionic acid;
methyl 3-[5-(5-carbamimidoyl-1H-benzoimidazol-2-yl)-5′-fluoro-6,2′-dihydroxy-biphenyl-3-yl]-propionate;
methyl 2-[5-(5-carbamimidoyl-1H-benzoimidazol-2-yl)-5′-fluoro-6,2′-dihydroxy-biphenyl-3-yl]-acetate;
2-[5-(5-carbamimidoyl-1H-benzoimidazol-2-yl)-6,2′-dihydroxy-biphenyl-3-yl]-acetic acid;
2-[5-(5-carbamimidoyl-1H-benzoimidazol-2-yl)-5′-fluoro-6,2′-dihydroxy-biphenyl-3-yl]-acetic acid;
2-[5-(5-N-hydroxycarbamimidoyl-1H-benzoimidazol-2-yl)-5′-fluoro-6,2′-dihydroxy-biphenyl-3-yl]-succinic acid;
diethyl 2-[5-(5-N-hydroxycarbamimidoyl-1H-benzoimidazol-2-yl)-5′-fluoro-6,2′-dihydroxy-biphenyl-3-yl]-succinate;
2-[5-(5-N-hydroxycarbamimidoyl-1H-benzoimidazol-2-yl)-6,2′-dihydroxy-5′-methylsulfonylaminobiphenyl-3-yl]-succinic acid;
diethyl 2-[5-(5-N-hydroxycarbamimidoyl-1H-benzoimidazol-2-yl)-6,2′-dihydroxy-5′-ureidomethylbiphenyl-3-yl]-succinate;
2-[5-(5-N-hydroxycarbamimidoyl-1H-benzoimidazol-2-yl)-5′-fluoro-6,2′-dihydroxy-biphenyl-3-yl]-acetic acid;
diethyl 2-[5-(5-N-hydroxycarbamimidoyl-1H-benzoimidazol-2-yl)-6,2′-dihydroxy-5′-hydroxymethylbiphenyl-3-yl]-succinate;
dimethyl 2-[5-(5-N-hydroxycarbamimidoyl-1H-benzoimidazol-2-yl)-5′-aminocarbonyl-6,2′-dihydroxybiphenyl-3-yl]-succinate; and
2-[5-(5-N-hydroxycarbamimidoyl-1H-benzoimidazol-2-yl)-6,2′-dihydroxy-5′-ureidomethylbiphenyl-3-yl]-succinic acid; or
a pharmaceutically acceptable salt thereof.

26. A compound selected from the group consisting of:
2-[5-(5-carbamimidoyl-1H-benzoimidazol-2-yl)-5′-fluoro-6,2′-dihydroxy-biphenyl-3-yl]-succinic acid;
2-[5-(5-carbamimidoyl-1H-benzoimidazol-2-yl)-6,2′-dihydroxy-5′-hydroxymethylbiphenyl-3-yl]-succinic acid;
2-[5-(5-carbamimidoyl-1H-benzoimidazol-2-yl)-6,2′-dihydroxy-5′-ureidomethyl-biphenyl-3-yl]-succinic acid;
2-[5-(5-carbamimidoyl-1H-benzoimidazol-2-yl)-5′-aminocarbonyl-6,2′-dihydroxy-biphenyl-3-yl]-succinic acid; and
2-[5-(5-carbamimidoyl-1H-benzoimidazol-2-yl)-3′-aminosulfonyl-6-hydroxy-biphenyl-3-yl]-succinic acid;
or a pharmaceutically acceptable salt thereof.

27. A pharmaceutical composition comprising a pharmaceutically acceptable carrier and a therapeutically effective amount of a compound of claim 1.

28. A pharmaceutical composition comprising a pharmaceutically acceptable carrier and a therapeutically effective amount of a compound of claim 25.

29. A method of treating a disease in an animal mediated by Factor VIIa which method comprises administering to said animal a pharmaceutical composition comprising a therapeutically effective amount of a compound of claim 1 and a pharmaceutically acceptable carrier.

30. A method of treating a disease in an animal mediated by Factor VIIa which method comprises administering to said animal a pharmaceutical composition comprising a therapeutically effective amount of a compound of claim 25 and a pharmaceutically acceptable carrier.

31. The method of claim 29 wherein the disorder is a thromboembolic disorder.

32. The method of claim 30 wherein the disorder is a thromboembolic disorder.

33. A method of treating a a thromboembolic disorder, which method comprises administering to said animal a pharmaceutical composition comprising a pharmaceutically acceptable carrier and a therapeutically effective amount of a compound of claim 1 in combination with another anticoagulant agent(s) independently selected from a group consisting of a thrombin inhibitor, a factor IXa, a factor Xa inhibitor, Aspirin®, and Plavis®.

34. A method for inhibiting the coagulation of a biological sample comprising the administration of a compound of claim 1.

35. An intermediate of Formula II: 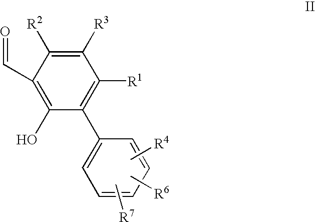
wherein R¹, R², R³, R⁴, R⁶, and R⁷ are as defined in claim 1.

36. A process of preparing a compound of claim 1 where X¹ is —N— comprising reacting a compound of Formula II: 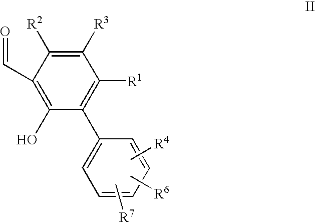
wherein R¹, R², R³, R⁴, R⁶, and R⁷ are as defined in claim 1 above, with a compound of Formula III: 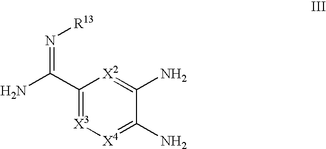
where R¹³ is hydrogen;
optionally modifying any of the R¹, R², R³, R⁴, R⁶, R⁷, and R¹³ groups;
optionally isolating individual isomers;
optionally preparing an acid addition salt; and
optionally preparing a free base.
optionally preparing an acid addition salt; and
optionally preparing a free base.
